SEQUENCE LISTING (626 sequences)

SEQ ID NO: 1 - protein, 348 aa, Homo sapiens
MSLMVVSMACVGFFLLQGAWPHEGVHRKPSLLAHPGRLVKSEETVILQCWSDVRFEHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHECRFSAGPKVNGTFQADFPLGPATHGGTYRCFGSFRDSPYEWSNSSDPLLVSVTGNPSNSWPSPTEPSSKTGNPRHLHILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCP

SEQ ID NO: 2 - DNA, 14488 bp, Homo sapiens
  source 1..14488: mol_type = other DNA
cgcggccgcctgtctgcacagacagcaccatgtcgctcatggtcgtcagcatggcgtgtgttggtgagtcctggaagggaatcgagggagggagtgcggggatggagatcggggcccagagttggagatataggcctggaagtggagttatgggcctagagatggagtgatgggcctagaagtggagatctgggcctggagtggagatatgggcctggaggttgagatatgggcctgcagtagagatatgggcttgtagtggagacatgggcctggagatggagatatgggcctggagatggagatatgggcctgcagtagagataggggcctggagtggagatatgggcctggagtggagatatgggcctgaagtggagatatgggcctggaggtggagatatgggcctggaggtggagatatgggcctggagtggagatatgggtctggaggtggagatacgggcctgcagtagagatatgggcctggagtggagatatgggccaggagtggagttatgggcctagaggtggatatctgggcctggagtggagatatgggcctaggaaggagatatgggcctgggtgtggagatatgggactggagaggtgatatgggcctggagtggagatatgggcttagggtggagttctgggcctggggcggagatatgggactggattggagataggggcctagggtggagatctgagcctggattggcgatatgggcctagggtggaaatatcagcctggagtggagatatgggcttggggtggggatatgggcctggaaactgggtctctgcacagccgacagccctgttcttgggtgcaggtaggcactgagggtgagtttaacttcagcccaggaagggcctggctgccaagactcacagcccagtgggggcagcaagggagggctggttcgcctgcagatggatcgtccatcatgatctttctttccagggttcttcttgctgcagggggcctggccacatgagggtgagtccttctccaaaccttcgggtgtcatctccccacataagaggattttcctgaaacaggagggaagtcctgtcggggagtctctcataaactaggaagagaggaccctggggtgctcagcccacatttctgacctcgcctccctggcctctcaaccccttggcagagtcaagttctgtggggaccagggttagactggggtgctcaaagctggggtgtgtggttgggaagtggtaggaacagcagatcctctgaggacaaaggtgttactcacacacttcagcgtttccatgatggtaggggctgcagtgtggctgctgtcattctaccagaagaggtgggaaaccacagccatggccctgacattccaaatcctctgatgggggctcagttgtttattttcgttcaggcatccgctgatatccattcacaaaggacatgccctccacctcatgtctaccctgtgttgttttatgtgagtaatcttacagtatcaaaatctagtaggagtctctttactcagcacttgctcaaagttctcagctgaggcttttgttgtagggagacaccatgtctttgcgggatgggtccttccttcagccctgggcaccaaggtgtgatagtagccatagaaacgtggaaagcgaggagaatcttctgagcacagggagggaggggcagttccacatcctcctctctaaggcggcgcctccttctccccaaggtggtcaggacaagcccttgctgtctgcctggcccagccttgtggtgcctctaggacatgtcattcttcggtgtcactcttatcttgggtttaacaacttcagtctgtacaaggaaggtggggtgcctgtccctgagctctacaacagaatattctggaacagccttttcatgggccctgtgacccccgcacaacagggacatacagatgtcggggttcacacacacactcccccagtgggtggtcagcacccagcaaccccctggtgatcgtggtcataggtcagagggctcctgtcttggattctccttgtcccacctcctgaatcccagagcttctggtgggcatgtccttgagggtcccatcacgcaggccctgactgtatttgtggtaaagggggattgaatacagggaaatgggtgctgtggtgggaagaataattgtccccagtgatgactacattctaatccctggagtctgtgactatgtatgttataggggaagggactgaaggggaagatggagctcatggggagacagcctggactgtcccactgggctcagtgtaatcacaagggtgcacatgaaaggaggaggaagaggggagtggggattagagcagtccagtggaagtcttcaccagctttgaaggtggaggaaggccaagagccatgaatgcaggtggcctatagaggctggaaaagtcaaggaactgattctccagagtctccagagggaacaaagccctgcagatgccttgattttagcccaggaaaaatagggtccaatttctgtctccagtactggaaggtgtcagtgtggtctctcctgcttccatgcttctgataattttgtacagcagcaacaggaaaccaacactggaacccaggtcaaggacaagttaagaaacaacccaaggaaagccaggcatggtggcaggcgcatgtaatcctagcgactcaggaggctgagggcaggagaatcacttgaacccaggaaacagaggttgcagtgagcctagaccacaccacttcactccagcctgggtgaaggagtgagactctgtctccaaaattaattaattaattaaagaaaccaaagaaggagaaggttggctaccctgagatcagcaagggtgggatgatgatgccaccaccaggctccatccacatagggaggggttgatactcctccaaccagcaccaggagccagcctatggaagctggcaccatggagaaggcacaggcatggcaagagtggctcccagtccccaccaggaacagggtgtgtggacactggtgcctgccttattcatcagttcatatcttctgccaaggattgcaattcatccaaaagagattgaaccaggctgataagagcctggatgtgcagcctatcctggttcctctttcacccccacataaacagcaggaaagacattagtgtgaaatagatacaacaccccaagagatgaggctaagcccagtgggaagggaatcagaggctactagagacagagggacagagaagagggagggagacagatggaaggacctgcaccaggagttaagggcacagaaaagaacatgaagacacagagaggaaggagagagacagacaccagcaaggggaagcctcactcattctaggtgccatggatgggatgataaagagagacaccttctaaactcacaacctctcttcctaggagtccacagaaaaccttccctcctggcccacccaggtcgcctggtgaaatcagaagagacagtcatcctgcaatgttggtcagatgtcaggtttgagcacttccttctgcacagagaagggaagtttaaggacactttgcacctcattggagagcaccatgatggggtctccaaagccaacttctccatcggtcccatgatgcaagaccttgcagggacctacagatgctacggttctgttactcactccccctatcagttgtcagctcccagtgaccctctggacatcgtcatcacaggtgagagtgtccggacattctcattgtcattgggctgcagagtgaatgatccacgacttggaacccccaggtagttgtaaggaagatgagcttggtattcttatggagagagactgacttgctgaggtttgtaccaacagagacagagaaacaggagacacaagtacagaccaggtgtcataacggaggacagacacaggggccatacagggagttagaaaagacagaaagagttaaaagagacagacagacagacatgtcccagagagaggtgtccctccatgctgactttgctcacagacctggcacaggttagaagtttcatttctgttttacctccacaaagtgttctctaccaggagaacccaaggacacccatatttctgacctgagttgggccctgtggcctcaggccttgtggcacctacaggccatgtttattctgacacctctgccttccatgtaatggagagtaaccgtcccaggatatcatggccccagaacaccaacccctgtatgctgtgtgaacttgtggtctccagactggattctgaggctcacattccaaataaccccacatatgaaaggatcactgagaggcacagagaaaaatcaggaacaccaaaaagcaaagacataaacacacggagaatgagccagaggaaggagattgagagactcacagacacataaagagagagaaaagagggcagaggagtggtgagaatgatggcagggagcagagaaaagcactaaaattagagtcctgagagagaggcacaaggacatagaaacatggagatgtggggatgaattgcagagattccaaagagagctagagagaccgagaggcagagcaatacagatgatagatggatagatatagatagatgataaataggtagatgatagataataggttaaagatacatagatgatgattgattgattcattaatagataatacatagagatgatgatgatgaagacagataatacgtacagatagagaggcagacagaaatcatagagagagagatgatacatacatataaataacagatgattgatggatagatagacaactgatagatacatagatgatatatagatatagatgacaggtagagaatttgtagataggcaccgaatagataaatagatagatcgacagataatagatagaaatatgcagaaagttatgaacaggacacaacgtgagaaacttagaatttaaaaaagtaacatcaagtcaaccaacccaaggagagtcagagagaataaaacaatccaaaaacggaaaacatatctagaggtggggaagcgaggtcagagacctagagagacagagaaggtggaagaaggaaatagatatgaagagagatggggtggagggtgagagagagagagagagagcattaggtcatagagcaggggagtgagttctcagctcaggtgaagggagctgtgacaaggaagatcctccctgaggaaaatgcctcttctccttccaggtctatatgagaaaccttctctctcagcccagccgggccccacggttctggcaggagagagcgtgaccttgtcctgcagctcccggagctcctatgacatgtaccatctatccagggagggggaggcccatgaatgtaggttctctgcagggcccaaggtcaacggaacattccaggccgactttcctctgggccctgccacccacggaggaacctacagatgcttcggctctttccgtgactctccatacgagtggtcaaactcgagtgacccactgcttgtttctgtcataggtgaggaaaccccatatctgtctcatgtcctatgatcctagagccttagctgaggagcttcctgctgatgatggagataagcatggacagatgcagagagaagacgaagcttgggtgtgagggagggatcagggcacaggatggcagacagggcacctccaaaccctcctacacggcctgcatgaaggcccgcggccagggctccaggcacacaggcagatggagaaagcggtcaggagagacccagaggagggagactgggctcagtttgggaagatcagaggttccctcagcccctcaacattacccatttcccagaagcccatcctggcctctcacccacacagggatgtcatcaccagcaacccctacaccctttacttttgtttgaagaaatatttattgaggataaatatacctatatagcttaccacctttaacattttttttttttttgaggcagagtctagctctgtcccctatgctgcagtgcagtggcacaatctcagctcactgcaacttccgcctcctgggttcaagtgattctcctgcctcagccacctgagtagctggtgctacaggcgcgcaccaccacgccaggctactttttgtatttttagtagagaggtggtttcaccatgttggtcgagctggtctccaactcctgaccacgtgatccacccgcatctgcctcccaaagtgctgggattacaggcatgagccaccactcccagccacatttaccatttttaagtgtaaagtctagtggtcataaatacatttataaatatatatatatatatatgtatgtatatatatatatatatatatatatatatatatatttttttttttttttttaccctccacccttttcttcctggcctctggaagccaccattctactctctaccttcatgagatccaccttttagctctgtatatgggtgagaaatgggaatctttgtaatgacttgcagttccatccatgtggctgcaaatatcaggatgttattctttctatggatgagtagtctccactgtgcgtatgtactacattctctctatccattcatccactgatgggcaggtaggttgactccacatcttggctactgtgaacagtgctgcaccaatcatacgagtgcagatatcacttcgatatattgatttactttcctttggatataaacccagtagtgaaattgctggatactatgaaagttctctttttagttattcgtttgttgttttgtttttgtttttgagacagtttccctctgtgcccaggctggagtacaagtgatgtcatcttggctcattgcaacctctgcctcctgggttcaaatgattttcctacctcagcctccctagtagctgggattacaggtgcacgccaccatgcctggctactttttggtttttttagtatagatggggtttccccatgttggctgggctgctctcaaactcatgacctcaactgaggtgtccgcctcggtctcccaaagtgccgggattacaggcatgatccacctcacccaacctctttttagttctttaaaggacttccacacttttctccgtaaaggctgtactaatttacactcctaccaacagggtattagggttctcctttctctaccactttggcaggatttcctttgcctgtcttgcagctaaaagccattttactttatttcattttattttgagatggagtttcgctcttgtcacccaggctggagtgcagtggtgcgatctcggctcaccacaacctccacctcccaggttcaagcgattctcctgcctcagcctcccgagtagctggaattacaggcacacgccaccacgcccgactaatttttgtatttttagtagagacagtgtttctccatgtgggtcagactggtctcaaactcccgaccttatgagattcacccacctcaggctctcaaagatctaggatgacagacgtgagccaccacgcccggcctaaaagccattttaatggggtgagatgaaaactcactttgattttaatttgcgtttctctgatgatgagtgatactgagcagtttttcgtatgtggggaaatttcatgtcttttgctcctgtttcaattaaatcatttgttttattgagttgtttgagcttcttatatttctagttattaatcccatctcagatgcatagtttgcacatatttgctcccaatctgtgggttgtctcttcactttgttggtttatttttagcggtgcagaagttgcttagcttgaggtaatcccaatggtctatttttgcttcgattacttgtgttttgaaggtttaaaacaaaatgtcttccttcagacaaatgtcctggagcatttccccaatattttcttctacgtgtttcataggttcaggccttagactcacatctttaatccattttcatttgatttttgtgtatggtgacaggtagaggtgcagtttcattcctctgcatgtagatgtccaggtttccctgcactgtttattgaaaagactgtcctttcctgattgtgagttcttggcacctttgtcaaagtccattggatgggctgggcatggtgactgacacctgcaatttcagcactttgggagcccaaggcgggtggatcacctgaggccaggagttcaagattagtctggccgacgtgatgaaacatcgtctccactaaaaatatataaattagctgagcatggtggtcagcacctataataccactactcaggagtttgaggccagagaattgattgaacccaggaggctgtggtggcagtgaaccgagattgcacctctgcactccagcctgggtgacagagcgagactccatctcaaaagaaaaaagaaaaaaacattggatgtaaatgcatggattatatttgtgttgttcattctgctccattgttctatgtgcctttcttcatgccaacatcatgctgtcttgcttactacagctctgtaacatattttgagatcaggtagtgtgatgctcctgttttctctttataccttgaagtctcaagacaatgggcgtcacatacaaaaattatggaaaaaaggatcccaggactcccagggcccaatattagataacagagtgttggccatgaaccaacctcaaagatttccattgagtagaggacagacaccctcatttcctcacctctctcctgtctcatgttctaggaaacccttcaaatagttggccttcacccactgaaccaagctctaaaaccggtgagtacagaaccctcttatatccgcttttggaaacctggggaggtagaaaccttcgatgcaggcattgactcagcatctcgcagctctgacattgtacgcctgtcttctaccatctccgaactccagatactccaacagcgaaagggatctgggcccaacctagggctcagtgaaatctcttaatctctcattttatggagctgagacctcctacaagctagaagaatgattgccaatctgacatccttctcaggaaaaatgcaatgtttgttctgcctgcattcctaactggaggataaattcctgggggcttgagagagggaagggaagggaacatctgatgagggcgaggtgttttagagaagttccacttgccaaggaatgaattactgttggtcatgaagcaaccctggctgactcagcagagcaacagccttgccgtaacagagaacggagctcatgcacgcacacttcgactcactgactcattcagccacggccccatgctcaggctgtgcagtgcggaaccttttcctattgttgccataacaaatttccacaagattcgtgggtgaaaacaaaacggttttttaattatcttacagtgctgtagctcaaagtaggaagtgcatcttactgggctaaaatcaaggtgacagcaaggctgccttccctctgaggattccaggcaagaatctgcttctcacttgtcccagcttctaaaggctcccagttccttggctcctggtccccttcctccttcctcaaaacccacaaagactggtcacatctcacatggcatcactcagtgccttcttccttaccacacctctttctctgaatgctgctctcccttcttccttatcttttgaaaacttggggattctattgggttcaccaagatgaaaatccctcataatctcctggaaatcatccaggatacccttgttttaagttcagctgattagcaaccgtaattccatctacaatcttcattcctcctttccatgtaaaataacatattcacaaggtatggaggctaggacagggacattttggggtgggacagcattctcctgccttccacaaacagtgaacaagatgcatttggcctctgcccttgggacactgatattgcagatggttaaatgggagggcagaaaatgaatgcacaagtggatctataaatgaatgatccattgggaagcatctgtgcatgaaatctattttttgtttgttcttttgtttattgagacagagttgccctctgtcttccaggctacagtgcagtgtcacgatcttggctcactgcaacctgcttctcctggattcaagtgattctcctgcctccgcctctcgagtagctgggattacaggcaactgccaccgtgcccggctaattctttttgtatattttttgtagagaggatgtttcaccacgttggccaagcttgtctgaaactcccaacctcaagtgatccgaccgtctcagcatgccaaagtaatgggactacaggcgtgagccactgtgcccagccagaattcaaaatcaataatagataatgctgagtgtatgatttcaggtgacaaagaaggtctcactattcagatatttgtgacattaatgaaaaacacggattgaacccctgaaagattggcggaaggattttgcacacacagctgtcagccgtgaaggcacaaaggtgaaaacaatctgatgtggaaggaagaggctcttcctcaaatgctgggaatgatgtggggagaatgacaagatgactgtggagagacggagagcacactgggtacacaggaaactaaggaggaacaaggagtgtgtgtttgacactcacagccattggattcacctcggggtagccaggaatccctacatgattaatatgactgacatgaaaataagggaggctcagttgcataactggaatctaggagaccgtggaaaaggcaattgccgccccactggtgaaatgtggtgctgatttagacactaaatgaatgaagtagatggatataagataggtttgtgaggtagaatcattgactggaaaggcttgctgggtttgattttcctacttgtttaatcctcgcttaattaatttctttctgagatttattcatcctacacataaatcaatacctggcaaaggagtgacagatatatgaggggtggtggaaatgaagagacctattatagcataatatacaagtctgtgaacggtggctcacgcctgtaacccagcactgcaggaggccaaggcgggtggatcacatgaagtcagcagttcgagaccagcctggccaacatggtgaaaccctgtctctaggaaaaacacaaaaattagccgagcatggtggtgcatccctgtaatcccagctcctactctggaggatgaagcaggagaatgacttcaacccaggaggtggaggttgcagtgagtggaggttgcatcactgcactccagcctgggtggcacaaggagactccgtctcaaaaaataaaaataagaaatgcataaatataaatataatataacacacgcaaatgacaaagggacctgaattccaatcatgatttttctatttctctataattacttctttgatcctttatcttatccattaggcaatgagcctaaaacctcttccctatttggctttctgtgagcatgagatcatatagaaaatgtgaaagtccgctgaatcctccagcacagatcctggaatagagaaagtgctctggtcatcacaaaaaaaacttgcccactcacccaaatcccccacctcacccctacttccaatcacctgtggagattcaggtagaccatggggaggtaaacattaacactccttggagtgagtccagatcttggaatcagagatcagcgacagcactagctcctgctcccctttcctactaattcacaggaggacaggtggtattgaagcaatagatggccgagggggtggtccttcccccagcctctcgggtagaacagcagcctaatatgtgtctcccgagatcacaaagagcagcaggtttcacacgggcttcaacactatttcctggccgtttgacataagagaattctatttcgctttttttatcttgatttcacttttgttttctttccttggagaatgcaagttgtttgattcaagaatgctgtggatgtagaaaccctaaagcacattcgctgtgaatcaatcccagtccagtcttcccagagaagactctaaacacctcctggactgcacctgggcctatgccaattcctatcactcaccgtcactccagggagacagaacacacagagaatacgttacataggcaggttcattactaacagataagcagcgagtgacaacagaaacctatatttcaatgtgagccagtccctcaaggctcagaaaagctcctcgggacatatggagtcaccccatttgcagtgtagctgcgggaagccagaaagcagcccagcctgggttttgtaccctggagccacaggaagcactcagctaaagcactgcatgacgtcctccaggaagaacaggaagacagcccagggtgttctgagacgttcctcctgatctcaggaagttgctgtcttaggccatttttgttgctctaaaggaacacttgagcctcggtaacttctaaagaaaagagattggtttgcctcaccgttctgcaggctgtactggaagcatggcaccagcatctatttctcgtgacggcctcaggctgctcccactctggcagaagggaaggagggtctgtctgtgcagagaccacagagatcacacggcaagagagggagcaagggggagggggagtgatggagcttccaagctctttttaacaaccagctctccgggaactaatagagggggaacttgctaaccccgtctccttgggacagcattgatgtgttcatgatggatccacctccatgacccaaacacctctcaagaggcccaacctcccacagtgggggtgaaatttcaatgtgaggtttgaaggggtcaaacatctcaactaaagtagtcgtatcctcagcacgttctatggttactatgagagctataactgaaaaagcaggagaaagctgggtctcctgccatctgggtgcttgtcctaaagagatgttttatgtggttacctgtcaatcaagaaatgcgagacaattcataaagaggaactgctaagattagcttcttattggtgtctcatcttcttccaggtaacccccgacacctgcacattctgattgggacctcagtggtcatcatcctcttcatcctcctcttctttctccttcatcgctggtgctccaacaaaaaaagtaagtctcacgaagcagaggccagagagctcagggccatgtggggaagcaggatgggagcactcaggtgtgtgttcctcacaaacaggatggtccctggcccaaggcagcagccacagaggcaggactttctagagagggcaccagactccctgcccctgccttcaactcacagaccgttgcctgattctgaactgtatcctcatgtcccctgcagccactcacatccaggagaaggttccatgacaggcagaaagtgggagacagaatcaatgggatgggaactcagagctattcatgggatgggtccttgagctcagagagatagaatgtctgagtctgctgttggcaactgagggacctcagccacctatggtctccccctgtatgttggtatctgcttatgaaatgaggacccagaagtgccctccgagctgttttgttgacttccgtctcctacagatgctgcggtaatggaccaagagtctgcagggaacagaacagcgaatagcgaggtaggtactcctcggcccgggctcgtggctactgttattcccaaagagtcctggaaaatgtgagcaccctccctcactcagcatttccctctctccaggactctgatgaacaagaccctcaggaggtgacatacacacagttgaatcactgcgttttcacacagagaaaaatcactcgcccttctcagaggcccaagacacccccaacagatatcatcgtgtacgcggaacttccaaatgctgagtccagatccaaagttgtctcctgcccatgagcaccacagtcaggccttgagggcgtcttctagggagacaacagccctgtctcaaaaccgggttgccagctcccatgtaccagcagctggaatctgaaggcatgagtctgcatcttagggcatcgctcttcctcacaccacaaatctgaatgtgcctctcacttgcttacaaatgtctaaggtccccactgcctgctggagaaaaaacacactcctttgcttagcccacagttctccatttcacttgacccctgcccacctctccaacctaactggcttacttcctagtctacttgaggctgcaatcacactgaggaactcacaattccaaacatacaagaggctccctcttaacgcagcacttagacacgtgttgttccaccttccctcatgctgttccacctcccctcagactagctttcagtcttctgtcagcagtaaaacttatatattttttaaaataacttcaatgtagttttccatccttcaaataaacatgtctgcccccatg

SEQ ID NO: 3 - DNA, 14670 bp, Homo sapiens
  source 1..14670: mol_type = other DNA
gagcacccactgggcctcatgcaaggtagaaagagcctgcgtacgtcaccctcccatgatgtggtcaacatgtaaactgcatgggcagggcgccaaataacatcctgtgcgctgctgagctgagctggggcgcggccgcctgtctgcacagacagcaccatgtcgctcatggtcgtcagcatggcgtgtgttggtgagtcctggaagggaatcgagggagggagtgcggggatggagatcggggcccagagttggagatataggcctggaagtggagttatgggcctagagatggagtgatgggcctagaagtggagatctgggcctggagtggagatatgggcctggaggttgagatatgggcctgcagtagagatatgggcttgtagtggagacatgggcctggagatggagatatgggcctggagatggagatatgggcctgcagtagagataggggcctggagtggagatatgggcctggagtggagatatgggcctgaagtggagatatgggcctggaggtggagatatgggcctggaggtggagatatgggcctggagtggagatatgggtctggaggtggagatacgggcctgcagtagagatatgggcctggagtggagatatgggccaggagtggagttatgggcctagaggtggatatctgggcctggagtggagatatgggcctaggaaggagatatgggcctgggtgtggagatatgggactggagaggtgatatgggcctggagtggagatatgggcttagggtggagttctgggcctggggcggagatatgggactggattggagataggggcctagggtggagatctgagcctggattggcgatatgggcctagggtggaaatatcagcctggagtggagatatgggcttggggtggggatatgggcctggaaactgggtctctgcacagccgacagccctgttcttgggtgcaggtaggcactgagggtgagtttaacttcagcccaggaagggcctggctgccaagactcacagcccagtgggggcagcaagggagggctggttcgcctgcagatggatcgtccatcatgatctttctttccagggttcttcttgctgcagggggcctggccacatgagggtgagtccttctccaaaccttcgggtgtcatctccccacataagaggattttcctgaaacaggagggaagtcctgtcggggagtctctcataaactaggaagagaggaccctggggtgctcagcccacatttctgacctcgcctccctggcctctcaaccccttggcagagtcaagttctgtggggaccagggttagactggggtgctcaaagctggggtgtgtggttgggaagtggtaggaacagcagatcctctgaggacaaaggtgttactcacacacttcagcgtttccatgatggtaggggctgcagtgtggctgctgtcattctaccagaagaggtgggaaaccacagccatggccctgacattccaaatcctctgatgggggctcagttgtttattttcgttcaggcatccgctgatatccattcacaaaggacatgccctccacctcatgtctaccctgtgttgttttatgtgagtaatcttacagtatcaaaatctagtaggagtctctttactcagcacttgctcaaagttctcagctgaggcttttgttgtagggagacaccatgtctttgcgggatgggtccttccttcagccctgggcaccaaggtgtgatagtagccatagaaacgtggaaagcgaggagaatcttctgagcacagggagggaggggcagttccacatcctcctctctaaggcggcgcctccttctccccaaggtggtcaggacaagcccttgctgtctgcctggcccagccttgtggtgcctctaggacatgtcattcttcggtgtcactcttatcttgggtttaacaacttcagtctgtacaaggaaggtggggtgcctgtccctgagctctacaacagaatattctggaacagccttttcatgggccctgtgacccccgcacaacagggacatacagatgtcggggttcacacacacactcccccagtgggtggtcagcacccagcaaccccctggtgatcgtggtcataggtcagagggctcctgtcttggattctccttgtcccacctcctgaatcccagagcttctggtgggcatgtccttgagggtcccatcacgcaggccctgactgtatttgtggtaaagggggattgaatacagggaaatgggtgctgtggtgggaagaataattgtccccagtgatgactacattctaatccctggagtctgtgactatgtatgttataggggaagggactgaaggggaagatggagctcatggggagacagcctggactgtcccactgggctcagtgtaatcacaagggtgcacatgaaaggaggaggaagaggggagtggggattagagcagtccagtggaagtcttcaccagctttgaaggtggaggaaggccaagagccatgaatgcaggtggcctatagaggctggaaaagtcaaggaactgattctccagagtctccagagggaacaaagccctgcagatgccttgattttagcccaggaaaaatagggtccaatttctgtctccagtactggaaggtgtcagtgtggtctctcctgcttccatgcttctgataattttgtacagcagcaacaggaaaccaacactggaacccaggtcaaggacaagttaagaaacaacccaaggaaagccaggcatggtggcaggtgcatgtaatcctagcgactcaggaggctgagggcaggagaatcacttgaacccaggaaacagaggttgcagtgagcctagaccacaccacttcactccagcctgggtgaaggagtgagactctgtctccaaaattaattaattaattaaagaaaccaaagaaggagaaggttggctaccctgagatcagcaagggtgggatgatgatgccaccaccaggctccatccacatagggaggggttgatactcctccaaccagcaccaggagccagcctatggaagctggcaccatggagaaggcacaggcatggcaagagtggctcccagtccccaccaggaacagggtgtgtggacactggtgcctgccttattcatcagttcatatcttctgccaaggattgcaattcatccaaaagagattgaaccaggctgataagagcctggatgtgcagcctatcctggttcctctttcacccccacataaacagcaggaaagacattagtgtgaaatagatacaacaccccaagagatgaggctaagcccagtgggaagggaatcagaggctactagagacagagggacagagaagagggagggagacagatggaaggacctgcaccaggagttaagggcacagaaaagaacatgaagacacagagaggaaggagagagacagacaccagcaaggggaagcctcactcattctaggtgccatggatgggatgataaagagagacaccttctaaactcacaacctctcttcctaggagtccacagaaaaccttccctcctggcccacccaggtcgcctggtgaaatcagaagagacagtcatcctgcaatgttggtcagatgtcaggtttgagcacttccttctgcacagagaagggaagtttaaggacactttgcacctcattggagagcaccatgatggggtctccaaagccaacttctccatcggtcccatgatgcaagaccttgcagggacctacagatgctacggttctgttactcactccccctatcagttgtcagctcccagtgaccctctggacatcgtcatcacaggtgagagtgtccggacattctcattgtcattgggctgcagagtgaatgatccacgacttggaacccccaggtagttgtaaggaagatgagcttggtattcttatggagagagactgacttgctgaggtttgtaccaacagagacagagaaacaggagacacaagtacagaccaggtgtcataacggaggacagacacaggggccatacagggagttagaaaagacagaaagagttaaaagagacagacagacagacatgtcccagagagaggtgtccctccatgctgactttgctcacagacctggcacaggttagaagtttcatttctgttttacctccacaaagtgttctctaccaggagaacccaaggacacccatatttctgacctgagttgggccctgtggcctcaggccttgtggcacctacaggccatgtttattctgacacctctgccttccatgtaatggagagtaaccgtcccaggatatcatggccccagaacaccaacccctgtatgctgtgtgaacttgtggtctccagactggattctgaggctcacattccaaataaccccacatatgaaaggatcactgagaggcacagagaaaaatcaggaacaccaaaaagcaaagacataaacacacggagaatgagccagaggaaggagattgagagactcacagacacataaagagagagaaaagagggcagaggagtggtgagaatgatggcagggagcagagaaaagcactaaaattagagtcctgagagagaggcacaaggacatagaaacatggagatgtggggatgaattgcagagattccaaagagagctagagagaccgagaggcagagcaatacagatgatagatggatagatatagatagatgataaataggtagatgatagataataggttaaagatacatagatgatgattgattgattcattaatagataatacatagagatgatgatgatgaagacagataatacgtacagatagagaggcagacagaaatcatagagagagagatgatacatacatataaataacagatgattgatggatagatagacaactgatagatacatagatgatatatagatatagatgacaggtagagaatttgtagataggcaccgaatagataaatagatagatcgacagataatagatagaaatatgcagaaagttatgaacaggacacaacgtgagaaacttagaatttaaaaaagtaacatcaagtcaaccaatccaaggagagtcagagagaataaaacaatccaaaaacggaaaacatatctagaggtggggaagcgaggtcagagacctagagagacagagaaggtggaagaaggaaatagacatgaagagagatggggtggagggtgagagagagagagagagagcattaggtcatagagcaggggagtgagttctcagctcaggtgaagggagctgtgacaaggaagatcctccctgaggaaaatgcctcttctccttccaggtctatatgagaaaccttctctctcagcccagccgggccccacggttctggcaggagagagcgtgaccttgtcctgcagctcccggagctcctatgacatgtaccatctatccagggagggggaggcccatgaatgtaggttctctgcagggcccaaggtcaacggaacattccaggccgactttcctctgggccctgccacccacggaggaacctacagatgcttcggctctttccgtgactctccatacgagtggtcaaactcgagtgacccactgcttgtttctgtcacaggtgaggaaaccccatatctgtctcatgtcctatgatcctagagccttagctgaggagcttcctgctgatgatggagataagcatggacagatgcagagagaagacgaagcttgggtgtgagggagggatcagggcacaggatggcagacagggcacctccaaaccctcctacacggcctgcatgaaggcccgcggccagggctccaggcacacaggcagatggagaaagcggtcaggagagacccagaggagggagactgggctcagtttgggaagatcagaggttccctcagcccctcaacattacccatttcccagaagcccatcctggcctctcacccacacagggatgtcatcaccagcaacccctacaccctttacttttgtttgaagaaatatttattgaggataaatatacctatatagcttaccacctttaacattttttttttttttgaggcagagtctagctctgtcccctatgctgcagtgcagtggcacaatctcagctcactgcaacttccgcctcctgggttcaagtgattctcctgcctcagccacctgagtagctggtgctacaggcgcgcaccaccacgccaggctactttttgtatttttagtagagaggtggtttcaccatgttggtcgagctggtctccaactcctgaccacgtgatccacccgcatctgcctcccaaagtgctgggattacaggcatgagccaccactcccagccacatttaccatttttaagtgtaaagtctagtggtcataaatacatttataaatatatatatatatatatatgtatgtatatatatatacacacacatatatatacatatatatatgtgtatatatatatatatatatatatatatatatatatatatatatttttttttttttttttaccctccacccttttcttcctggcctctggaagccaccattctactctctaccttcatgagatccaccttttagctctgtatatgggtgagaaatgggaatctttgtaatgacttccagttccatccatgtggctgcaaatatcaggatgttattctttctatggatgagtagtctccactgtgcgtatgtactacattctctctatccattcatccactgatgggcaggtaggttgactccacatcttggctactgtgaacagtgctgcaccaatcatacgagtgcagatatcacttcgatatattgatttactttcctttggatataaacccagtagtgaaattgctggatactatgaaagttctctttttagttattcgtttgttgttttgtttttgtttttgagacagtttccctctgtgcccaggctggagtacaagtgatgtcatcttggctcattgcaacctctgcctcctgggttcaaatgattttcctacctcagcctccctagtagctgggattacaggtgcacgccaccatgcctggctactttttggtttttttagtatagatggggtttccccatgttggctgggctgctctcaaactcatgacctcaactgaggtgtccgcctcggtctcccaaagtgccgggattacaggcatgatccacctcacccaacctctttttagttctttaaaggacttccacacttttctccgtaaaggctgtactaatttacactcctaccaacagggtattagggttctcctttctctaccactttggcaggatttcctttgcctgtcttgcagctaaaagccattttactttatttcattttattttgagatggagtttcgctcttgtcacccaggctggagtgcagtggtgcgatctcggctcaccacaacctccacctcccaggttcaagcgattctcctgcctcagcctcccgagtagctggaattacaggcacacgccaccacgcccgactaatttttgtatttttagtagagacagtgtttctccatgtgggtcagactggtctcaaactcccgaccttatgagattcacccacctcaggctctcaaagatctaggatgacagacgtgagccaccacgcccggcctaaaagccattttaatggggtgagatgaaaactcactttgattttaatttgcgtttctctgatgatgagtgatactgagcagtttttcgtatgtggggaaatttcatgtcttttgctcctgtttcaattaaatcatttgttttattgagttgtttgagcttcttatatttctagttattaatcccatctcagatgcatagtttgcacatatttgctcccaatctgtgggttgtctcttcactttgttggtttatttttagcggtgcagaagttgcttagcttgaggtaatcccaatggtctatttttgcttcgattacttgtgttttgaaggtttaaaacaaaatgtcttccttcagacaaatgtcctggagcatttccccaatattttcttctacgtgtttcataggttcaggccttagactcacatctttaatccattttcatttgatttttgtgtatggtgacaggtagaggtgcagtttcattcctctgcatgtagatgtccaggtttccctgcactgtttattgaaaagactgtcctttcctgattgtgagttcttggcacctttgtcaaagtccattggatgggctgggcatggtgactgacacctgcaatttcagcactttgggagcccaaggcgggtggatcacctgaggccaggagttcaagattagtctggccgacgtgatgaaacattgtctccactaaaaatatataaattagctgagcatggtggtcagcacctataataccactactcaggagtttgaggccagagaattgattgaacccaggaggctgtggtggcagtgaaccgagattgcacctctgcactccagcctgggtgacagagcgagactccatctcaaaagaaaaaagaaaaaaacattggatgtaaatgcatggattatatttgtgttgttcattctgctccattgttctatgtgcctttcttcatgccaacatcatgctgtcttgcttactacagctctgtaacatattttgagatcaggtagtgtgatgctcctgttttctctttataccttgaagtctcaagacaatgggcgtcacatacaaaaattatggaaaaaaggatcccaggactcccagggcccaatattagataacagagtgttggccatgaaccaacctcaaagatttccattgagtagaggacagacaccctcatttcctcacctctctcctgtctcatgttctaggaaacccttcaaatagttggccttcacccactgaaccaagctctaaaaccggtgagtacagaaccctcttatatccgcttttggaaacctggggaggtagaaaccttcgatgcaggcattgactcagcatctcgcagctctgacattgtacgcctgtcttctaccatctccgaactccagatactccaacagcgaaagggatctgggcccaacctagggctcagtgaaatctcttaatctctcattttatggagctgagacctcctacaagctagaagaatgattgccaatctgacatccttctcaggaaaaatgcaatgtttgttctgcctgcattcctaactggaggataaattcctgggggcttgagagagggaagggaagggaacatctgatgagggcgaggtgttttagagaagttccacttgccaaggaatgaattactgttggtcatgaagcaaccctggctgactcagcagagcaacagccttgccgtaacagagaacggagctcatgcacgcacacttcgactcactgactcattcagccacggccccatgctcaggctgtgcagtgcggaaccttttcctattgttgccataacaaatttccacaagattcgtgggtgaaaacaaaacggttttttaattatcttacagtgctgtagctcaaagtaggaagtgcatcttactgggctaaaatcaaggtgacagcaaggctgccttccctctgaggattccaggcaagaatctgcttctcacttgtcccagcttctaaaggctcccagttccttggctcctggtccccttcctccttcctcaaaacccacaaagactggtcacatctcacatggcatcactcagtgccttcttccttaccacacctctttctctgaatgctgctctcccttcttccttatcttttgaaaacttggggattctattgggttcaccaagatgaaaatccctcataatctcctggaaatcatccaggatacccttgttttaagttcagctgattagcaaccgtaattccatctacaatcttcattcctcctttccatgtaaaataacatattcacaaggtatggaggctaggacagggacattttggggtgggacagcattctcctgccttccacaaacagtgaacaagatgcatttggcctctgcccttgggacactgatattgcagatggttaaatgggagggcagaaaatgaatgcacaagtggatctataaatgaatgatccattgggaagcatctgtgcatgaaatctattttttgtttgttcttttgtttattgagacagagttgccctctgtcttccaggctacagtgcagtgtcacgatcttggctcactgcaacctgcttctcctggattcaagtgattctcctgcctccgcctctcgagtagctgggattacaggcaactgccaccgtgcccggctaattctttttgtatattttttgtagagaggatgtttcaccacgttggccaagcttgtctgaaactcccaacctcaagtgatccgaccgtctcagcatgccaaagtaatgggactacaggcgtgagccactgtgcccagccagaattcaaaatcaataatagataatgctgagtgtatgatttcaggtgacaaagaaggtctcactattcagatatttgtgacattaatgaaaaacacggattgaacccctgaaagattggcggaaggattttgcacacacagctgtcagccgtgaaggcacaaaggtgaaaacaatctgatgtggaaggaagaggctcttcctcaaatgctgggaatgatgtggggagaatgacaagatgactgtggagagacggagagcacactgggtacacaggaaactaaggaggaacaaggagtgtgtgtttgacactcacagccattggattcacctcggggtagccaggaatccctacatgattaatatgactgacatgaaaataagggaggctcagttgcataactggaatctaggagaccgtggaaaaggcaattgccgccccactggtgaaatgtggtgctgatttagacactaaatgaatgaagtagatggatataagataggtttgtgaggtagaatcattgactggaaaggcttgctgggtttgattttcctacttgtttaatcctcgcttaattaatttctttctgagatttattcatcctacacataaatcaatacctggcaaaggagtgacagatatatgaggggtggtggaaatgaagagacctattatagcataatatacaagtctgtgaacggtggctcacgcctgtaacccagcactgcaggaggccaaggcgggtggatcacatgaagtcagcagttcgagaccagcctggccaacatggtgaaaccctgtctctaggaaaaacacaaaaattagccgagcatggtggtgcatccctgtaatcccagctcctactctggaggatgaagcaggagaatgacttcaacccaggaggtggaggttgcagtgagtggaggttgcatcactgcactccagcctgggtggcacaaggagactccgtctcaaaaaataaaaataagaaatgcataaatataaatataatataacacacgcaaatgacaaagggacctgaattccaatcatgatttttctatttctctataattacttctttgatcctttatcttatccattaggcaatgagcctaaaacctcttccctatttggctttctgtgagcatgagatcatatagaaaatgtgaaagtccgctgaatcctccagcacagatcctggaatagagaaagtgctctggtcatcacaaaaaaaacttgcccactcacccaaatcccccacctcacccctacttccaatcacctgtggagattcaggtagaccatggggaggtaaacattaacactccttggagtgagtccagatcttggaatcagagatcagcgacagcactagctcctgctcccctttcctactaattcacaggaggacaggtggtattgaagcaatagatggccgagggggtggtccttcccccagcctctcgggtagaacagcagcctaatatgtgtctcccgagatcacaaagagcagcaggtttcacacgggcttcaacactatttcctggccgtttgacataagagaattctatttcgctttttttatcttgatttcacttttgttttctttccttggagaatgcaagttgtttgattcaagaatgctgtggatgtagaaaccctaaagcacattcgctgtgaatcaatcccagtccagtcttcccagagaagactctaaacacctcctggactgcacctgggcctatgccaattcctatcactcaccgtcactccagggagacagaacacacagagaatacgttacataggcaggttcattactaacagataagcagcgagtgacaacagaaacctatatttcaatgtgagccagtccctcaaggctcagaaaagctcctcgggacatatggagtcaccccatttgcagtgtagctgcgggaagccagaaagcagcccagcctgggttttgtaccctggagccacaggaagcactcagctaaagcactgcatgacgtcctccaggaagaacaggaagacagcccagggtgttctgagacgttcctcctgatctcaggaagttgctgtcttaggccatttttgttgctctaaaggaacacttgagcctcggtaacttctaaagaaaagagattggtttgcctcaccgttctgcaggctgtactggaagcatggcaccagcatctatttctcgtgacggcctcaggctgctcccactctggcagaagggaaggagggtctgtctgtgcagagaccacagagatcacacggcaagagagggagcaagggggagggggagtgatggagcttccaagctctttttaacaaccagctctccgggaactaatagagggggaacttgctaaccccgtctccttgggacagcattgatgtgttcatgatggatccacctccatgacccaaacacctctcaagaggcccaacctcccacagtgggggtgaaatttcaatgtgaggtttgaaggggtcaaacatctcaactaaagtagtcgtatcctcagcacgttctatggttactatgagagctataactgaaaaagcaggagaaagctgggtctcctgccatctgggtgcttgtcctaaagagatgttttatgtggttacctgtcaatcaagaaatgcgagacaattcataaagaggaactgctaagattagcttcttattggtgtctcatcttcttccaggtaacccccgacacctgcacattctgattgggacctcagtggtcatcatcctcttcatcctcctcttctttctccttcatcgctggtgctccaacaaaaaaagtaagtctcacgaagcagaggccagagagctcagggccatgtggggaagcaggatgggagcactcaggtgtgtgttcctcacaaacaggatggtccctggcccaaggcagcagccacagaggcaggactttctagagagggcaccagactccctgcccctgccttcaactcacagaccgttgcctgattctgaactgtatcctcatgtcccctgcagccactcacatccaggagaaggttccatgacaggcagaaagtgggagacagaatcaatgggatgggaactcagagctattcatgggatgggtccttgagctcagagagatagaatgtctgagtctgctgttggcaactgagggacctcagccacctatggtctccccctgtatgttggtatctgcttatgaaatgaggacccagaagtgccctccgagctgttttgttgacttccgtctcctacagatgctgcggtaatggaccaagagtctgcagggaacagaacagcgaatagcgaggtaggtactcctcggcccgggctcgtggctactgttattcccaaagagtcctggaaaatgtgagcaccctccctcactcagcatttccctctctccaggactctgatgaacaagaccctcaggaggtgacatacacacagttgaatcactgcgttttcacacagagaaaaatcactcgcccttctcagaggcccaagacacccccaacagatatcatcgtgtacacggaacttccaaatgctgagtccagatccaaagttgtctcctgcccatgagcaccacagtcaggccttgagggcgtcttctagggagacaacagccctgtctcaaaaccgggttgccagctcccatgtaccagcagctggaatctgaaggcatgagtctgcatcttagggcatcgctcttcctcacaccacaaatctgaatgtgcctctcacttgcttacaaatgtctaaggtccccactgcctgctggagaaaaaacacactcctttgcttagcccacagttctccatttcacttgacccctgcccacctctccaacctaactggcttacttcctagtctacttgaggctgcaatcacactgaggaactcacaattccaaacatacaagaggctccctcttaacgcagcacttagacacgtgttgttccaccttccctcatgctgttccacctcccctcagactagctttcagtcttctgtcagcagtaaaacttatatattttttaaaataacttcaatgtagttttccatccttcaaataaacatgtctgcccccatg

SEQ ID NO: 4 - DNA, 1587 bp, Homo sapiens
  source 1..1587: mol_type = other DNA
cgcggccgcctgtctgcacagacagcaccatgtcgctcatggtcgtcagcatggcgtgtgttgggttcttcttgctgcagggggcctggccacatgagggagtccacagaaaaccttccctcctggcccacccaggtcgcctggtgaaatcagaagagacagtcatcctgcaatgttggtcagatgtcaggtttgagcacttccttctgcacagagaagggaagtttaaggacactttgcacctcattggagagcaccatgatggggtctccaaagccaacttctccatcggtcccatgatgcaagaccttgcagggacctacagatgctacggttctgttactcactccccctatcagttgtcagctcccagtgaccctctggacatcgtcatcacaggtctatatgagaaaccttctctctcagcccagccgggccccacggttctggcaggagagagcgtgaccttgtcctgcagctcccggagctcctatgacatgtaccatctatccagggagggggaggcccatgaatgtaggttctctgcagggcccaaggtcaacggaacattccaggccgactttcctctgggccctgccacccacggaggaacctacagatgcttcggctctttccgtgactctccatacgagtggtcaaactcgagtgacccactgcttgtttctgtcacaggaaacccttcaaatagttggccttcacccactgaaccaagctctaaaaccggtaacccccgacacctgcacattctgattgggacctcagtggtcatcatcctcttcatcctcctcttctttctccttcatcgctggtgctccaacaaaaaaaatgctgcggtaatggaccaagagtctgcagggaacagaacagcgaatagcgaggactctgatgaacaagaccctcaggaggtgacatacacacagttgaatcactgcgttttcacacagagaaaaatcactcgcccttctcagaggcccaagacacccccaacagatatcatcgtgtacacggaacttccaaatgctgagtccagatccaaagttgtctcctgcccatgagcaccacagtcaggccttgagggcgtcttctagggagacaacagccctgtctcaaaaccgggttgccagctcccatgtaccagcagctggaatctgaaggcatgagtctgcatcttagggcatcgctcttcctcacaccacaaatctgaatgtgcctctcacttgcttacaaatgtctaaggtccccactgcctgctggagaaaaaacacactcctttgcttagcccacagttctccatttcacttgacccctgcccacctctccaacctaactggcttacttcctagtctacttgaggctgcaatcacactgaggaactcacaattccaaacatacaagaggctccctcttaacgcagcacttagacacgtgttgttccaccttccctcatgctgttccacctcccctcagactagctttcagtcttctgtcagcagtaaaacttatatattttttaaaataacttcaatgtagttttccatccttcaaataaacatgtctgcccccatg

SEQ ID NO: 5 - DNA, 20 bp, synthetic construct
  misc_feature 1..20: note = Synthetic Construct
  source 1..20: mol_type = other DNA
ctcttgagcgagcacccact

SEQ ID NO: 6 - DNA, 20 bp, synthetic construct
  misc_feature 1..20: note = Synthetic Construct
  source 1..20: mol_type = other DNA
gctcttgagcgagcacccac

SEQ ID NO: 7 - DNA, 20 bp, synthetic construct
  misc_feature 1..20: note = Synthetic Construct
  source 1..20: mol_type = other DNA
agtgggtgctcgctcaagag

SEQ ID NO: 8 - DNA, 20 bp, synthetic construct
  misc_feature 1..20: note = Synthetic Construct
  source 1..20: mol_type = other DNA
gctcgctcaagagcggaaca

SEQ ID NO: 9 - DNA, 20 bp, synthetic construct
  misc_feature 1..20: note = Synthetic Construct
  source 1..20: mol_type = other DNA
taccttgcatgaggcccagt

SEQ ID NO: 10 - DNA, 20 bp, synthetic construct
  misc_feature 1..20: note = Synthetic Construct
  source 1..20: mol_type = other DNA
tggtcaacatgtaaactgca

SEQ ID NO: 11 - DNA, 20 bp, synthetic construct
  misc_feature 1..20: note = Synthetic Construct
  source 1..20: mol_type = other DNA
tgtgcgctgctgagctgagc

SEQ ID NO: 12 - DNA, 20 bp, synthetic construct
  misc_feature 1..20: note = Synthetic Construct
  source 1..20: mol_type = other DNA
ggtcaacatgtaaactgcat

SEQ ID NO: 13 - DNA, 20 bp, synthetic construct
  misc_feature 1..20: note = Synthetic Construct
  source 1..20: mol_type = other DNA
catgggagggtgacgtacgc

SEQ ID NO: 14 - DNA, 20 bp, synthetic construct
  misc_feature 1..20: note = Synthetic Construct
  source 1..20: mol_type = other DNA
atgttgaccacatcatggga

SEQ ID NO: 15 - DNA, 20 bp, synthetic construct
  misc_feature 1..20: note = Synthetic Construct
  source 1..20: mol_type = other DNA
tttacatgttgaccacatca

SEQ ID NO: 16 - DNA, 20 bp, synthetic construct
  misc_feature 1..20: note = Synthetic Construct
  source 1..20: mol_type = other DNA
acatgtaaactgcatgggca

SEQ ID NO: 17 - DNA, 20 bp, synthetic construct
  misc_feature 1..20: note = Synthetic Construct
  source 1..20: mol_type = other DNA
gcagcgcacaggatgttatt

SEQ ID NO: 18 - DNA, 20 bp, synthetic construct
  misc_feature 1..20: note = Synthetic Construct
  source 1..20: mol_type = other DNA
tgcgctgctgagctgagctg

SEQ ID NO: 19 - DNA, 20 bp, synthetic construct
  misc_feature 1..20: note = Synthetic Construct
  source 1..20: mol_type = other DNA
aacatgtaaactgcatgggc

SEQ ID NO: 20 - DNA, 20 bp, synthetic construct
  misc_feature 1..20: note = Synthetic Construct
  source 1..20: mol_type = other DNA
agctcagctcagcagcgcac

SEQ ID NO: 21 - DNA, 20 bp, synthetic construct
  misc_feature 1..20: note = Synthetic Construct
  source 1..20: mol_type = other DNA
ttacatgttgaccacatcat

SEQ ID NO: 22 - DNA, 20 bp, synthetic construct
  misc_feature 1..20: note = Synthetic Construct
  source 1..20: mol_type = other DNA
gtgcgctgctgagctgagct

SEQ ID NO: 23 - DNA, 20 bp, synthetic construct
  misc_feature 1..20: note = Synthetic Construct
  source 1..20: mol_type = other DNA
tacgtcaccctcccatgatg

SEQ ID NO: 24 - DNA, 20 bp, synthetic construct
  misc_feature 1..20: note = Synthetic Construct
  source 1..20: mol_type = other DNA
catgttgaccacatcatggg

SEQ ID NO: 25 - DNA, 20 bp, synthetic construct
  misc_feature 1..20: note = Synthetic Construct
  source 1..20: mol_type = other DNA
ggctctttctaccttgcatg

SEQ ID NO: 26 - DNA, 20 bp, synthetic construct
  misc_feature 1..20: note = Synthetic Construct
  source 1..20: mol_type = other DNA
ctaccttgcatgaggcccag

SEQ ID NO: 27 - DNA, 20 bp, synthetic construct
  misc_feature 1..20: note = Synthetic Construct
  source 1..20: mol_type = other DNA
cacccactgggcctcatgca

SEQ ID NO: 28 - DNA, 20 bp, synthetic construct
  misc_feature 1..20: note = Synthetic Construct
  source 1..20: mol_type = other DNA
agacagcaccatgtcgctca

SEQ ID NO: 29 - DNA, 20 bp, synthetic construct
  misc_feature 1..20: note = Synthetic Construct
  source 1..20: mol_type = other DNA
atggtgctgtctgtgcagac

SEQ ID NO: 30 - DNA, 20 bp, synthetic construct
  misc_feature 1..20: note = Synthetic Construct
  source 1..20: mol_type = other DNA
gtgctgtctgtgcagacagg

SEQ ID NO: 31 - DNA, 20 bp, synthetic construct
  misc_feature 1..20: note = Synthetic Construct
  source 1..20: mol_type = other DNA
gctgacgaccatgagcgaca

SEQ ID NO: 32 - DNA, 20 bp, synthetic construct
  misc_feature 1..20: note = Synthetic Construct
  source 1..20: mol_type = other DNA
gtcgctcatggtcgtcagca

SEQ ID NO: 33 - DNA, 20 bp, synthetic construct
  misc_feature 1..20: note = Synthetic Construct
  source 1..20: mol_type = other DNA
cagggggcctggccacatga

SEQ ID NO: 34 - DNA, 20 bp, synthetic construct
  misc_feature 1..20: note = Synthetic Construct
  source 1..20: mol_type = other DNA
ggactcaccctcatgtggcc

SEQ ID NO: 35 - DNA, 20 bp, synthetic construct
  misc_feature 1..20: note = Synthetic Construct
  source 1..20: mol_type = other DNA
cagggttcttcttgctgcag

SEQ ID NO: 36 - DNA, 20 bp, synthetic construct
  misc_feature 1..20: note = Synthetic Construct
  source 1..20: mol_type = other DNA
ccagggttcttcttgctgca

SEQ ID NO: 37 - DNA, 20 bp, synthetic construct
  misc_feature 1..20: note = Synthetic Construct
  source 1..20: mol_type = other DNA
tccagggttcttcttgctgc

SEQ ID NO: 38 - DNA, 20 bp, synthetic construct
  misc_feature 1..20: note = Synthetic Construct
  source 1..20: mol_type = other DNA
tcttcttgctgcagggggcc

SEQ ID NO: 39 - DNA, 20 bp, synthetic construct
  misc_feature 1..20: note = Synthetic Construct
  source 1..20: mol_type = other DNA
agggttcttcttgctgcagg

SEQ ID NO: 40 - DNA, 20 bp, synthetic construct
  misc_feature 1..20: note = Synthetic Construct
  source 1..20: mol_type = other DNA
gcagggggcctggccacatg

SEQ ID NO: 41 - DNA, 20 bp, synthetic construct
  misc_feature 1..20: note = Synthetic Construct
  source 1..20: mol_type = other DNA
ccctgcagcaagaagaaccc

SEQ ID NO: 42 - DNA, 20 bp, synthetic construct
  misc_feature 1..20: note = Synthetic Construct
  source 1..20: mol_type = other DNA
cctgcaaggtcttgcatcat

SEQ ID NO: 43 - DNA, 20 bp, synthetic construct
  misc_feature 1..20: note = Synthetic Construct
  source 1..20: mol_type = other DNA
cccatgatgcaagaccttgc

SEQ ID NO: 44 - DNA, 20 bp, synthetic construct
  misc_feature 1..20: note = Synthetic Construct
  source 1..20: mol_type = other DNA
ccctgcaaggtcttgcatca

SEQ ID NO: 45 - DNA, 20 bp, synthetic construct
  misc_feature 1..20: note = Synthetic Construct
  source 1..20: mol_type = other DNA
ccatgatgcaagaccttgca

SEQ ID NO: 46 - DNA, 20 bp, synthetic construct
  misc_feature 1..20: note = Synthetic Construct
  source 1..20: mol_type = other DNA
aaggacactttgcacctcat

SEQ ID NO: 47 - DNA, 20 bp, synthetic construct
  misc_feature 1..20: note = Synthetic Construct
  source 1..20: mol_type = other DNA
gtcttgcatcatgggaccga

SEQ ID NO: 48 - DNA, 20 bp, synthetic construct
  misc_feature 1..20: note = Synthetic Construct
  source 1..20: mol_type = other DNA
gcacagagaagggaagttta

SEQ ID NO: 49 - DNA, 20 bp, synthetic construct
  misc_feature 1..20: note = Synthetic Construct
  source 1..20: mol_type = other DNA
accgatggagaagttggctt

SEQ ID NO: 50 - DNA, 20 bp, synthetic construct
  misc_feature 1..20: note = Synthetic Construct
  source 1..20: mol_type = other DNA
tctgatttcaccaggcgacc

SEQ ID NO: 51 - DNA, 20 bp, synthetic construct
  misc_feature 1..20: note = Synthetic Construct
  source 1..20: mol_type = other DNA
ctgtgatgacgatgtccaga

SEQ ID NO: 52 - DNA, 20 bp, synthetic construct
  misc_feature 1..20: note = Synthetic Construct
  source 1..20: mol_type = other DNA
catcatggtgctctccaatg

SEQ ID NO: 53 - DNA, 20 bp, synthetic construct
  misc_feature 1..20: note = Synthetic Construct
  source 1..20: mol_type = other DNA
cctggcccacccaggtcgcc

SEQ ID NO: 54 - DNA, 20 bp, synthetic construct
  misc_feature 1..20: note = Synthetic Construct
  source 1..20: mol_type = other DNA
tgcaatgttggtcagatgtc

SEQ ID NO: 55 - DNA, 20 bp, synthetic construct
  misc_feature 1..20: note = Synthetic Construct
  source 1..20: mol_type = other DNA
ctgatttcaccaggcgacct

SEQ ID NO: 56 - DNA, 20 bp, synthetic construct
  misc_feature 1..20: note = Synthetic Construct
  source 1..20: mol_type = other DNA
tcattggagagcaccatgat

SEQ ID NO: 57 - DNA, 20 bp, synthetic construct
  misc_feature 1..20: note = Synthetic Construct
  source 1..20: mol_type = other DNA
ctcattggagagcaccatga

SEQ ID NO: 58 - DNA, 20 bp, synthetic construct
  misc_feature 1..20: note = Synthetic Construct
  source 1..20: mol_type = other DNA
cattggagagcaccatgatg

SEQ ID NO: 59 - DNA, 20 bp, synthetic construct
  misc_feature 1..20: note = Synthetic Construct
  source 1..20: mol_type = other DNA
tttcaccaggcgacctgggt

SEQ ID NO: 60 - DNA, 20 bp, synthetic construct
  misc_feature 1..20: note = Synthetic Construct
  source 1..20: mol_type = other DNA
atttcaccaggcgacctggg

SEQ ID NO: 61 - DNA, 20 bp, synthetic construct
  misc_feature 1..20: note = Synthetic Construct
  source 1..20: mol_type = other DNA
tccaaagccaacttctccat

SEQ ID NO: 62 - DNA, 20 bp, synthetic construct
  misc_feature 1..20: note = Synthetic Construct
  source 1..20: mol_type = other DNA
acttccttctgcacagagaa

SEQ ID NO: 63 - DNA, 20 bp, synthetic construct
  misc_feature 1..20: note = Synthetic Construct
  source 1..20: mol_type = other DNA
acttcccttctctgtgcaga

SEQ ID NO: 64 - DNA, 20 bp, synthetic construct
  misc_feature 1..20: note = Synthetic Construct
  source 1..20: mol_type = other DNA
ccaggcgacctgggtgggcc

SEQ ID NO: 65 - DNA, 20 bp, synthetic construct
  misc_feature 1..20: note = Synthetic Construct
  source 1..20: mol_type = other DNA
gacgatgtccagagggtcac

SEQ ID NO: 66 - DNA, 20 bp, synthetic construct
  misc_feature 1..20: note = Synthetic Construct
  source 1..20: mol_type = other DNA
tggctttggagaccccatca

SEQ ID NO: 67 - DNA, 20 bp, synthetic construct
  misc_feature 1..20: note = Synthetic Construct
  source 1..20: mol_type = other DNA
agcatctgtaggtccctgca

SEQ ID NO: 68 - DNA, 20 bp, synthetic construct
  misc_feature 1..20: note = Synthetic Construct
  source 1..20: mol_type = other DNA
catgggaccgatggagaagt

SEQ ID NO: 69 - DNA, 20 bp, synthetic construct
  misc_feature 1..20: note = Synthetic Construct
  source 1..20: mol_type = other DNA
gggagctgacaactgatagg

SEQ ID NO: 70 - DNA, 20 bp, synthetic construct
  misc_feature 1..20: note = Synthetic Construct
  source 1..20: mol_type = other DNA
ctgtctcttctgatttcacc

SEQ ID NO: 71 - DNA, 20 bp, synthetic construct
  misc_feature 1..20: note = Synthetic Construct
  source 1..20: mol_type = other DNA
ctgggagctgacaactgata

SEQ ID NO: 72 - DNA, 20 bp, synthetic construct
  misc_feature 1..20: note = Synthetic Construct
  source 1..20: mol_type = other DNA
tgggagctgacaactgatag

SEQ ID NO: 73 - DNA, 20 bp, synthetic construct
  misc_feature 1..20: note = Synthetic Construct
  source 1..20: mol_type = other DNA
aacagaaccgtagcatctgt

SEQ ID NO: 74 - DNA, 20 bp, synthetic construct
  misc_feature 1..20: note = Synthetic Construct
  source 1..20: mol_type = other DNA
actgggagctgacaactgat

SEQ ID NO: 75 - DNA, 20 bp, synthetic construct
  misc_feature 1..20: note = Synthetic Construct
  source 1..20: mol_type = other DNA
gtcagctcccagtgaccctc

SEQ ID NO: 76 - DNA, 20 bp, synthetic construct
  misc_feature 1..20: note = Synthetic Construct
  source 1..20: mol_type = other DNA
acgatgtccagagggtcact

SEQ ID NO: 77 - DNA, 20 bp, synthetic construct
  misc_feature 1..20: note = Synthetic Construct
  source 1..20: mol_type = other DNA
ggcgacctgggtgggccagg

SEQ ID NO: 78 - DNA, 20 bp, synthetic construct
  misc_feature 1..20: note = Synthetic Construct
  source 1..20: mol_type = other DNA
gcgacctgggtgggccagga

SEQ ID NO: 79 - DNA, 20 bp, synthetic construct
  misc_feature 1..20: note = Synthetic Construct
  source 1..20: mol_type = other DNA
tgacatctgaccaacattgc

SEQ ID NO: 80 - DNA, 20 bp, synthetic construct
  misc_feature 1..20: note = Synthetic Construct
  source 1..20: mol_type = other DNA
gcagggacctacagatgcta

SEQ ID NO: 81 - DNA, 20 bp, synthetic construct
  misc_feature 1..20: note = Synthetic Construct
  source 1..20: mol_type = other DNA
agacagtcatcctgcaatgt

SEQ ID NO: 82 - DNA, 20 bp, synthetic construct
  misc_feature 1..20: note = Synthetic Construct
  source 1..20: mol_type = other DNA
cacttccttctgcacagaga

SEQ ID NO: 83 - DNA, 20 bp, synthetic construct
  misc_feature 1..20: note = Synthetic Construct
  source 1..20: mol_type = other DNA
ccacagaaaaccttccctcc

SEQ ID NO: 84 - DNA, 20 bp, synthetic construct
  misc_feature 1..20: note = Synthetic Construct
  source 1..20: mol_type = other DNA
ccttccctcctggcccaccc

SEQ ID NO: 85 - DNA, 20 bp, synthetic construct
  misc_feature 1..20: note = Synthetic Construct
  source 1..20: mol_type = other DNA
ccaggagggaaggttttctg

SEQ ID NO: 86 - DNA, 20 bp, synthetic construct
  misc_feature 1..20: note = Synthetic Construct
  source 1..20: mol_type = other DNA
cctgggtgggccaggaggga

SEQ ID NO: 87 - DNA, 20 bp, synthetic construct
  misc_feature 1..20: note = Synthetic Construct
  source 1..20: mol_type = other DNA
ctgcagagaacctacattca

SEQ ID NO: 88 - DNA, 20 bp, synthetic construct
  misc_feature 1..20: note = Synthetic Construct
  source 1..20: mol_type = other DNA
agggggaggcccatgaatgt

SEQ ID NO: 89 - DNA, 20 bp, synthetic construct
  misc_feature 1..20: note = Synthetic Construct
  source 1..20: mol_type = other DNA
tgcagagaacctacattcat

SEQ ID NO: 90 - DNA, 20 bp, synthetic construct
  misc_feature 1..20: note = Synthetic Construct
  source 1..20: mol_type = other DNA
catgaatgtaggttctctgc

SEQ ID NO: 91 - DNA, 20 bp, synthetic construct
  misc_feature 1..20: note = Synthetic Construct
  source 1..20: mol_type = other DNA
atgaatgtaggttctctgca

SEQ ID NO: 92 - DNA, 20 bp, synthetic construct
  misc_feature 1..20: note = Synthetic Construct
  source 1..20: mol_type = other DNA
tccgtgactctccatacgag

SEQ ID NO: 93 - DNA, 20 bp, synthetic construct
  misc_feature 1..20: note = Synthetic Construct
  source 1..20: mol_type = other DNA
gctctctcctgccagaaccg

SEQ ID NO: 94 - DNA, 20 bp, synthetic construct
  misc_feature 1..20: note = Synthetic Construct
  source 1..20: mol_type = other DNA
agcatctgtaggttcctccg

SEQ ID NO: 95 - DNA, 20 bp, synthetic construct
  misc_feature 1..20: note = Synthetic Construct
  source 1..20: mol_type = other DNA
taggttctctgcagggccca

SEQ ID NO: 96 - DNA, 20 bp, synthetic construct
  misc_feature 1..20: note = Synthetic Construct
  source 1..20: mol_type = other DNA
ctcgagtttgaccactcgta

SEQ ID NO: 97 - DNA, 20 bp, synthetic construct
  misc_feature 1..20: note = Synthetic Construct
  source 1..20: mol_type = other DNA
accactcgtatggagagtca

SEQ ID NO: 98 - DNA, 20 bp, synthetic construct
  misc_feature 1..20: note = Synthetic Construct
  source 1..20: mol_type = other DNA
ctctctcctgccagaaccgt

SEQ ID NO: 99 - DNA, 20 bp, synthetic construct
  misc_feature 1..20: note = Synthetic Construct
  source 1..20: mol_type = other DNA
ggcccagaggaaagtcggcc

SEQ ID NO: 100 - DNA, 20 bp, synthetic construct
  misc_feature 1..20: note = Synthetic Construct
  source 1..20: mol_type = other DNA
ctggaatgttccgttgacct

SEQ ID NO: 101 - DNA, 20 bp, synthetic construct
  misc_feature 1..20: note = Synthetic Construct
  source 1..20: mol_type = other DNA
tggaatgttccgttgacctt

SEQ ID NO: 102 - DNA, 20 bp, synthetic construct
  misc_feature 1..20: note = Synthetic Construct
  source 1..20: mol_type = other DNA
tctgcagggcccaaggtcaa

SEQ ID NO: 103 - DNA, 20 bp, synthetic construct
  misc_feature 1..20: note = Synthetic Construct
  source 1..20: mol_type = other DNA
gcatctgtaggttcctccgt

SEQ ID NO: 104 - DNA, 20 bp, synthetic construct
  misc_feature 1..20: note = Synthetic Construct
  source 1..20: mol_type = other DNA
ggatagatggtacatgtcat

SEQ ID NO: 105 - DNA, 20 bp, synthetic construct
  misc_feature 1..20: note = Synthetic Construct
  source 1..20: mol_type = other DNA
caaggtcaacggaacattcc

SEQ ID NO: 106 - DNA, 20 bp, synthetic construct
  misc_feature 1..20: note = Synthetic Construct
  source 1..20: mol_type = other DNA
ccgggccccacggttctggc

SEQ ID NO: 107 - DNA, 20 bp, synthetic construct
  misc_feature 1..20: note = Synthetic Construct
  source 1..20: mol_type = other DNA
atgacatgtaccatctatcc

SEQ ID NO: 108 - DNA, 20 bp, synthetic construct
  misc_feature 1..20: note = Synthetic Construct
  source 1..20: mol_type = other DNA
tctctcctgccagaaccgtg

SEQ ID NO: 109 - DNA, 20 bp, synthetic construct
  misc_feature 1..20: note = Synthetic Construct
  source 1..20: mol_type = other DNA
ttcatgggcctccccctccc

SEQ ID NO: 110 - DNA, 20 bp, synthetic construct
  misc_feature 1..20: note = Synthetic Construct
  source 1..20: mol_type = other DNA
tgtaccatctatccagggag

SEQ ID NO: 111 - DNA, 20 bp, synthetic construct
  misc_feature 1..20: note = Synthetic Construct
  source 1..20: mol_type = other DNA
ataggagctccgggagctgc

SEQ ID NO: 112 - DNA, 20 bp, synthetic construct
  misc_feature 1..20: note = Synthetic Construct
  source 1..20: mol_type = other DNA
attccaggccgactttcctc

SEQ ID NO: 113 - DNA, 20 bp, synthetic construct
  misc_feature 1..20: note = Synthetic Construct
  source 1..20: mol_type = other DNA
taggttcctccgtgggtggc

SEQ ID NO: 114 - DNA, 20 bp, synthetic construct
  misc_feature 1..20: note = Synthetic Construct
  source 1..20: mol_type = other DNA
ttccaggccgactttcctct

SEQ ID NO: 115 - DNA, 20 bp, synthetic construct
  misc_feature 1..20: note = Synthetic Construct
  source 1..20: mol_type = other DNA
atgtaccatctatccaggga

SEQ ID NO: 116 - DNA, 20 bp, synthetic construct
  misc_feature 1..20: note = Synthetic Construct
  source 1..20: mol_type = other DNA
tgacatgtaccatctatcca

SEQ ID NO: 117 - DNA, 20 bp, synthetic construct
  misc_feature 1..20: note = Synthetic Construct
  source 1..20: mol_type = other DNA
ccagaaccgtggggcccggc

SEQ ID NO: 118 - DNA, 20 bp, synthetic construct
  misc_feature 1..20: note = Synthetic Construct
  source 1..20: mol_type = other DNA
cctgccagaaccgtggggcc

SEQ ID NO: 119 - DNA, 20 bp, synthetic construct
  misc_feature 1..20: note = Synthetic Construct
  source 1..20: mol_type = other DNA
cagaaccgtggggcccggct

SEQ ID NO: 120 - DNA, 20 bp, synthetic construct
  misc_feature 1..20: note = Synthetic Construct
  source 1..20: mol_type = other DNA
ggaggaacctacagatgctt

SEQ ID NO: 121 - DNA, 20 bp, synthetic construct
  misc_feature 1..20: note = Synthetic Construct
  source 1..20: mol_type = other DNA
ccagccgggccccacggttc

SEQ ID NO: 122 - DNA, 20 bp, synthetic construct
  misc_feature 1..20: note = Synthetic Construct
  source 1..20: mol_type = other DNA
gtaccatctatccagggagg

SEQ ID NO: 123 - DNA, 20 bp, synthetic construct
  misc_feature 1..20: note = Synthetic Construct
  source 1..20: mol_type = other DNA
gtacatgtcataggagctcc

SEQ ID NO: 124 - DNA, 20 bp, synthetic construct
  misc_feature 1..20: note = Synthetic Construct
  source 1..20: mol_type = other DNA
catgtaccatctatccaggg

SEQ ID NO: 125 - DNA, 20 bp, synthetic construct
  misc_feature 1..20: note = Synthetic Construct
  source 1..20: mol_type = other DNA
ccatctatccagggaggggg

SEQ ID NO: 126 - DNA, 20 bp, synthetic construct
  misc_feature 1..20: note = Synthetic Construct
  source 1..20: mol_type = other DNA
ggtacatgtcataggagctc

SEQ ID NO: 127 - DNA, 20 bp, synthetic construct
  misc_feature 1..20: note = Synthetic Construct
  source 1..20: mol_type = other DNA
ggcagggcccagaggaaagt

SEQ ID NO: 128 - DNA, 20 bp, synthetic construct
  misc_feature 1..20: note = Synthetic Construct
  source 1..20: mol_type = other DNA
cctccccctccctggataga

SEQ ID NO: 129 - DNA, 20 bp, synthetic construct
  misc_feature 1..20: note = Synthetic Construct
  source 1..20: mol_type = other DNA
tctgtaggttcctccgtggg

SEQ ID NO: 130 - DNA, 20 bp, synthetic construct
  misc_feature 1..20: note = Synthetic Construct
  source 1..20: mol_type = other DNA
ctgtgacagaaacaagcagt

SEQ ID NO: 131 - DNA, 20 bp, synthetic construct
  misc_feature 1..20: note = Synthetic Construct
  source 1..20: mol_type = other DNA
gctccgggagctgcaggaca

SEQ ID NO: 132 - DNA, 20 bp, synthetic construct
  misc_feature 1..20: note = Synthetic Construct
  source 1..20: mol_type = other DNA
tgaccttgtcctgcagctcc

SEQ ID NO: 133 - DNA, 20 bp, synthetic construct
  misc_feature 1..20: note = Synthetic Construct
  source 1..20: mol_type = other DNA
aggttcctccgtgggtggca

SEQ ID NO: 134 - DNA, 20 bp, synthetic construct
  misc_feature 1..20: note = Synthetic Construct
  source 1..20: mol_type = other DNA
ctcagcccagccgggcccca

SEQ ID NO: 135 - DNA, 20 bp, synthetic construct
  misc_feature 1..20: note = Synthetic Construct
  source 1..20: mol_type = other DNA
ctgggccctgccacccacgg

SEQ ID NO: 136 - DNA, 20 bp, synthetic construct
  misc_feature 1..20: note = Synthetic Construct
  source 1..20: mol_type = other DNA
ccttctctctcagcccagcc

SEQ ID NO: 137 - DNA, 20 bp, synthetic construct
  misc_feature 1..20: note = Synthetic Construct
  source 1..20: mol_type = other DNA
gaaagagccgaagcatctgt

SEQ ID NO: 138 - DNA, 20 bp, synthetic construct
  misc_feature 1..20: note = Synthetic Construct
  source 1..20: mol_type = other DNA
accttctctctcagcccagc

SEQ ID NO: 139 - DNA, 20 bp, synthetic construct
  misc_feature 1..20: note = Synthetic Construct
  source 1..20: mol_type = other DNA
cccggctgggctgagagaga

SEQ ID NO: 140 - DNA, 20 bp, synthetic construct
  misc_feature 1..20: note = Synthetic Construct
  source 1..20: mol_type = other DNA
ccgtgggtggcagggcccag

SEQ ID NO: 141 - DNA, 20 bp, synthetic construct
  misc_feature 1..20: note = Synthetic Construct
  source 1..20: mol_type = other DNA
cctctgggccctgccaccca

SEQ ID NO: 142 - DNA, 20 bp, synthetic construct
  misc_feature 1..20: note = Synthetic Construct
  source 1..20: mol_type = other DNA
tactcaccggttttagagct

SEQ ID NO: 143 - DNA, 20 bp, synthetic construct
  misc_feature 1..20: note = Synthetic Construct
  source 1..20: mol_type = other DNA
actgaaccaagctctaaaac

SEQ ID NO: 144 - DNA, 20 bp, synthetic construct
  misc_feature 1..20: note = Synthetic Construct
  source 1..20: mol_type = other DNA
gttttagagcttggttcagt

SEQ ID NO: 145 - DNA, 20 bp, synthetic construct
  misc_feature 1..20: note = Synthetic Construct
  source 1..20: mol_type = other DNA
gttttagagcttggttcagt

SEQ ID NO: 146 - DNA, 20 bp, synthetic construct
  misc_feature 1..20: note = Synthetic Construct
  source 1..20: mol_type = other DNA
ggttttagagcttggttcag

SEQ ID NO: 147 - DNA, 20 bp, synthetic construct
  misc_feature 1..20: note = Synthetic Construct
  source 1..20: mol_type = other DNA
taggaaacccttcaaatagt

SEQ ID NO: 148 - DNA, 20 bp, synthetic construct
  misc_feature 1..20: note = Synthetic Construct
  source 1..20: mol_type = other DNA
taggaaacccttcaaatagt

SEQ ID NO: 149 - DNA, 20 bp, synthetic construct
  misc_feature 1..20: note = Synthetic Construct
  source 1..20: mol_type = other DNA
gagcttggttcagtgggtga

SEQ ID NO: 150 - DNA, 20 bp, synthetic construct
  misc_feature 1..20: note = Synthetic Construct
  source 1..20: mol_type = other DNA
tcagaatgtgcaggtgtcgg

SEQ ID NO: 151 - DNA, 20 bp, synthetic construct
  misc_feature 1..20: note = Synthetic Construct
  source 1..20: mol_type = other DNA
tcagaatgtgcaggtgtcgg

SEQ ID NO: 152 - DNA, 20 bp, synthetic construct
  misc_feature 1..20: note = Synthetic Construct
  source 1..20: mol_type = other DNA
cgacacctgcacattctgat

SEQ ID NO: 153 - DNA, 20 bp, synthetic construct
  misc_feature 1..20: note = Synthetic Construct
  source 1..20: mol_type = other DNA
tgcaggtgtcgggggttacc

SEQ ID NO: 154 - DNA, 20 bp, synthetic construct
  misc_feature 1..20: note = Synthetic Construct
  source 1..20: mol_type = other DNA
tgcaggtgtcgggggttacc

SEQ ID NO: 155 - DNA, 20 bp, synthetic construct
  misc_feature 1..20: note = Synthetic Construct
  source 1..20: mol_type = other DNA
aatcagaatgtgcaggtgtc

SEQ ID NO: 156 - DNA, 20 bp, synthetic construct
  misc_feature 1..20: note = Synthetic Construct
  source 1..20: mol_type = other DNA
aggtcccaatcagaatgtgc

SEQ ID NO: 157 - DNA, 20 bp, synthetic construct
  misc_feature 1..20: note = Synthetic Construct
  source 1..20: mol_type = other DNA
gacacctgcacattctgatt

SEQ ID NO: 158 - DNA, 20 bp, synthetic construct
  misc_feature 1..20: note = Synthetic Construct
  source 1..20: mol_type = other DNA
gacacctgcacattctgatt

SEQ ID NO: 159 - DNA, 20 bp, synthetic construct
  misc_feature 1..20: note = Synthetic Construct
  source 1..20: mol_type = other DNA
cattctgattgggacctcag

SEQ ID NO: 160 - DNA, 20 bp, synthetic construct
  misc_feature 1..20: note = Synthetic Construct
  source 1..20: mol_type = other DNA
cattctgattgggacctcag

SEQ ID NO: 161 - DNA, 20 bp, synthetic construct
  misc_feature 1..20: note = Synthetic Construct
  source 1..20: mol_type = other DNA
gtgagacttactttttttgt

SEQ ID NO: 162 - DNA, 20 bp, synthetic construct
  misc_feature 1..20: note = Synthetic Construct
  source 1..20: mol_type = other DNA
tcttctttctccttcatcgc

SEQ ID NO: 163 - DNA, 20 bp, synthetic construct
  misc_feature 1..20: note = Synthetic Construct
  source 1..20: mol_type = other DNA
gaaagaagaggaggatgaag

SEQ ID NO: 164 - DNA, 20 bp, synthetic construct
  misc_feature 1..20: note = Synthetic Construct
  source 1..20: mol_type = other DNA
agcgatgaaggagaaagaag

SEQ ID NO: 165 - DNA, 20 bp, synthetic construct
  misc_feature 1..20: note = Synthetic Construct
  source 1..20: mol_type = other DNA
agcgatgaaggagaaagaag

SEQ ID NO: 166 - DNA, 20 bp, synthetic construct
  misc_feature 1..20: note = Synthetic Construct
  source 1..20: mol_type = other DNA
tcctacagatgctgcggtaa

SEQ ID NO: 167 - DNA, 20 bp, synthetic construct
  misc_feature 1..20: note = Synthetic Construct
  source 1..20: mol_type = other DNA
tccattaccgcagcatctgt

SEQ ID NO: 168 - DNA, 20 bp, synthetic construct
  misc_feature 1..20: note = Synthetic Construct
  source 1..20: mol_type = other DNA
taatggaccaagagtctgca

SEQ ID NO: 169 - DNA, 20 bp, synthetic construct
  misc_feature 1..20: note = Synthetic Construct
  source 1..20: mol_type = other DNA
gaacagaacagcgaatagcg

SEQ ID NO: 170 - DNA, 20 bp, synthetic construct
  misc_feature 1..20: note = Synthetic Construct
  source 1..20: mol_type = other DNA
gaacagaacagcgaatagcg

SEQ ID NO: 171 - DNA, 20 bp, synthetic construct
  misc_feature 1..20: note = Synthetic Construct
  source 1..20: mol_type = other DNA
gtaatggaccaagagtctgc

SEQ ID NO: 172 - DNA, 20 bp, synthetic construct
  misc_feature 1..20: note = Synthetic Construct
  source 1..20: mol_type = other DNA
gtaatggaccaagagtctgc

SEQ ID NO: 173 - DNA, 20 bp, synthetic construct
  misc_feature 1..20: note = Synthetic Construct
  source 1..20: mol_type = other DNA
aacagatatcatcgtgtaca

SEQ ID NO: 174 - DNA, 20 bp, synthetic construct
  misc_feature 1..20: note = Synthetic Construct
  source 1..20: mol_type = other DNA
gtacacgatgatatctgttg

SEQ ID NO: 175 - DNA, 20 bp, synthetic construct
  misc_feature 1..20: note = Synthetic Construct
  source 1..20: mol_type = other DNA
gtgtacacgatgatatctgt

SEQ ID NO: 176 - DNA, 20 bp, synthetic construct
  misc_feature 1..20: note = Synthetic Construct
  source 1..20: mol_type = other DNA
tgtacacgatgatatctgtt

SEQ ID NO: 177 - DNA, 20 bp, synthetic construct
  misc_feature 1..20: note = Synthetic Construct
  source 1..20: mol_type = other DNA
tacacgatgatatctgttgg

SEQ ID NO: 178 - DNA, 20 bp, synthetic construct
  misc_feature 1..20: note = Synthetic Construct
  source 1..20: mol_type = other DNA
ttggatctggactcagcatt

SEQ ID NO: 179 - DNA, 20 bp, synthetic construct
  misc_feature 1..20: note = Synthetic Construct
  source 1..20: mol_type = other DNA
tgtgtgtatgtcacctcctg

SEQ ID NO: 180 - DNA, 20 bp, synthetic construct
  misc_feature 1..20: note = Synthetic Construct
  source 1..20: mol_type = other DNA
gtgtgtatgtcacctcctga

SEQ ID NO: 181 - DNA, 20 bp, synthetic construct
  misc_feature 1..20: note = Synthetic Construct
  source 1..20: mol_type = other DNA
gcaggagacaactttggatc

SEQ ID NO: 182 - DNA, 20 bp, synthetic construct
  misc_feature 1..20: note = Synthetic Construct
  source 1..20: mol_type = other DNA
tcatgggcaggagacaactt

SEQ ID NO: 183 - DNA, 20 bp, synthetic construct
  misc_feature 1..20: note = Synthetic Construct
  source 1..20: mol_type = other DNA
atatctgttgggggtgtctt

SEQ ID NO: 184 - DNA, 20 bp, synthetic construct
  misc_feature 1..20: note = Synthetic Construct
  source 1..20: mol_type = other DNA
gatatctgttgggggtgtct

SEQ ID NO: 185 - DNA, 20 bp, synthetic construct
  misc_feature 1..20: note = Synthetic Construct
  source 1..20: mol_type = other DNA
ggtcttgttcatcagagtcc

SEQ ID NO: 186 - DNA, 20 bp, synthetic construct
  misc_feature 1..20: note = Synthetic Construct
  source 1..20: mol_type = other DNA
ctctgatgaacaagaccctc

SEQ ID NO: 187 - DNA, 20 bp, synthetic construct
  misc_feature 1..20: note = Synthetic Construct
  source 1..20: mol_type = other DNA
aaatcactcgcccttctcag

SEQ ID NO: 188 - DNA, 20 bp, synthetic construct
  misc_feature 1..20: note = Synthetic Construct
  source 1..20: mol_type = other DNA
gtgtcttgggcctctgagaa

SEQ ID NO: 189 - DNA, 20 bp, synthetic construct
  misc_feature 1..20: note = Synthetic Construct
  source 1..20: mol_type = other DNA
tgatgaacaagaccctcagg

SEQ ID NO: 190 - DNA, 20 bp, synthetic construct
  misc_feature 1..20: note = Synthetic Construct
  source 1..20: mol_type = other DNA
ggtgtcttgggcctctgaga

SEQ ID NO: 191 - DNA, 20 bp, synthetic construct
  misc_feature 1..20: note = Synthetic Construct
  source 1..20: mol_type = other DNA
gtctaagtgctgcgttaaga

SEQ ID NO: 192 - DNA, 20 bp, synthetic construct
  misc_feature 1..20: note = Synthetic Construct
  source 1..20: mol_type = other DNA
tgtctaagtgctgcgttaag

SEQ ID NO: 193 - DNA, 20 bp, synthetic construct
  misc_feature 1..20: note = Synthetic Construct
  source 1..20: mol_type = other DNA
gagggagcctcttgtatgtt

SEQ ID NO: 194 - DNA, 20 bp, synthetic construct
  misc_feature 1..20: note = Synthetic Construct
  source 1..20: mol_type = other DNA
gcccacctctccaacctaac

SEQ ID NO: 195 - DNA, 20 bp, synthetic construct
  misc_feature 1..20: note = Synthetic Construct
  source 1..20: mol_type = other DNA
attgcagcctcaagtagact

SEQ ID NO: 196 - DNA, 20 bp, synthetic construct
  misc_feature 1..20: note = Synthetic Construct
  source 1..20: mol_type = other DNA
gactaggaagtaagccagtt

SEQ ID NO: 197 - DNA, 20 bp, synthetic construct
  misc_feature 1..20: note = Synthetic Construct
  source 1..20: mol_type = other DNA
aggaagtaagccagttaggt

SEQ ID NO: 198 - DNA, 20 bp, synthetic construct
  misc_feature 1..20: note = Synthetic Construct
  source 1..20: mol_type = other DNA
gtaagccagttaggttggag

SEQ ID NO: 199 - DNA, 20 bp, synthetic construct
  misc_feature 1..20: note = Synthetic Construct
  source 1..20: mol_type = other DNA
agactgaaagctagtctgag

SEQ ID NO: 200 - DNA, 20 bp, synthetic construct
  misc_feature 1..20: note = Synthetic Construct
  source 1..20: mol_type = other DNA
cttacttcctagtctacttg

SEQ ID NO: 201 - DNA, 20 bp, synthetic construct
  misc_feature 1..20: note = Synthetic Construct
  source 1..20: mol_type = other DNA
gaagactgaaagctagtctg

SEQ ID NO: 202 - DNA, 20 bp, synthetic construct
  misc_feature 1..20: note = Synthetic Construct
  source 1..20: mol_type = other DNA
ctccctagaagacgccctca

SEQ ID NO: 203 - DNA, 20 bp, synthetic construct
  misc_feature 1..20: note = Synthetic Construct
  source 1..20: mol_type = other DNA
ctgaaagctagtctgagggg

SEQ ID NO: 204 - DNA, 20 bp, synthetic construct
  misc_feature 1..20: note = Synthetic Construct
  source 1..20: mol_type = other DNA
ttgaggctgcaatcacactg

SEQ ID NO: 205 - DNA, 20 bp, synthetic construct
  misc_feature 1..20: note = Synthetic Construct
  source 1..20: mol_type = other DNA
aagactgaaagctagtctga

SEQ ID NO: 206 - DNA, 20 bp, synthetic construct
  misc_feature 1..20: note = Synthetic Construct
  source 1..20: mol_type = other DNA
gccagttaggttggagaggt

SEQ ID NO: 207 - DNA, 20 bp, synthetic construct
  misc_feature 1..20: note = Synthetic Construct
  source 1..20: mol_type = other DNA
aggccttgagggcgtcttct

SEQ ID NO: 208 - DNA, 20 bp, synthetic construct
  misc_feature 1..20: note = Synthetic Construct
  source 1..20: mol_type = other DNA
tgggcaggggtcaagtgaaa

SEQ ID NO: 209 - DNA, 20 bp, synthetic construct
  misc_feature 1..20: note = Synthetic Construct
  source 1..20: mol_type = other DNA
ggccttgagggcgtcttcta

SEQ ID NO: 210 - DNA, 20 bp, synthetic construct
  misc_feature 1..20: note = Synthetic Construct
  source 1..20: mol_type = other DNA
cacaattccaaacatacaag

SEQ ID NO: 211 - DNA, 20 bp, synthetic construct
  misc_feature 1..20: note = Synthetic Construct
  source 1..20: mol_type = other DNA
ggggaggtggaacagcatga

SEQ ID NO: 212 - DNA, 20 bp, synthetic construct
  misc_feature 1..20: note = Synthetic Construct
  source 1..20: mol_type = other DNA
aggggaggtggaacagcatg

SEQ ID NO: 213 - DNA, 20 bp, synthetic construct
  misc_feature 1..20: note = Synthetic Construct
  source 1..20: mol_type = other DNA
agccagttaggttggagagg

SEQ ID NO: 214 - DNA, 20 bp, synthetic construct
  misc_feature 1..20: note = Synthetic Construct
  source 1..20: mol_type = other DNA
aaagctagtctgaggggagg

SEQ ID NO: 215 - DNA, 20 bp, synthetic construct
  misc_feature 1..20: note = Synthetic Construct
  source 1..20: mol_type = other DNA
aggtggaacagcatgaggga

SEQ ID NO: 216 - DNA, 20 bp, synthetic construct
  misc_feature 1..20: note = Synthetic Construct
  source 1..20: mol_type = other DNA
tcaagtgaaatggagaactg

SEQ ID NO: 217 - DNA, 20 bp, synthetic construct
  misc_feature 1..20: note = Synthetic Construct
  source 1..20: mol_type = other DNA
caagtgaaatggagaactgt

SEQ ID NO: 218 - DNA, 20 bp, synthetic construct
  misc_feature 1..20: note = Synthetic Construct
  source 1..20: mol_type = other DNA
gttaggttggagaggtgggc

SEQ ID NO: 219 - DNA, 20 bp, synthetic construct
  misc_feature 1..20: note = Synthetic Construct
  source 1..20: mol_type = other DNA
tggaacagcatgagggaagg

SEQ ID NO: 220 - DNA, 20 bp, synthetic construct
  misc_feature 1..20: note = Synthetic Construct
  source 1..20: mol_type = other DNA
acgccctcaaggcctgactg

SEQ ID NO: 221 - DNA, 20 bp, synthetic construct
  misc_feature 1..20: note = Synthetic Construct
  source 1..20: mol_type = other DNA
gggcagacatgtttatttga

SEQ ID NO: 222 - DNA, 20 bp, synthetic construct
  misc_feature 1..20: note = Synthetic Construct
  source 1..20: mol_type = other DNA
taggttggagaggtgggcag

SEQ ID NO: 223 - DNA, 20 bp, synthetic construct
  misc_feature 1..20: note = Synthetic Construct
  source 1..20: mol_type = other DNA
agacatgtttatttgaagga

SEQ ID NO: 224 - DNA, 20 bp, synthetic construct
  misc_feature 1..20: note = Synthetic Construct
  source 1..20: mol_type = other DNA
gagtgtgttttttctccagc

SEQ ID NO: 225 - DNA, 20 bp, synthetic construct
  misc_feature 1..20: note = Synthetic Construct
  source 1..20: mol_type = other DNA
ttaggttggagaggtgggca

SEQ ID NO: 226 - DNA, 20 bp, synthetic construct
  misc_feature 1..20: note = Synthetic Construct
  source 1..20: mol_type = other DNA
gacatttgtaagcaagtgag

SEQ ID NO: 227 - DNA, 20 bp, synthetic construct
  misc_feature 1..20: note = Synthetic Construct
  source 1..20: mol_type = other DNA
acattcagatttgtggtgtg

SEQ ID NO: 228 - DNA, 20 bp, synthetic construct
  misc_feature 1..20: note = Synthetic Construct
  source 1..20: mol_type = other DNA
gagaggcacattcagatttg

SEQ ID NO: 229 - DNA, 20 bp, synthetic construct
  misc_feature 1..20: note = Synthetic Construct
  source 1..20: mol_type = other DNA
aaggcatgagtctgcatctt

SEQ ID NO: 230 - DNA, 20 bp, synthetic construct
  misc_feature 1..20: note = Synthetic Construct
  source 1..20: mol_type = other DNA
gttttttctccagcaggcag

SEQ ID NO: 231 - DNA, 20 bp, synthetic construct
  misc_feature 1..20: note = Synthetic Construct
  source 1..20: mol_type = other DNA
gagaactgtgggctaagcaa

SEQ ID NO: 232 - DNA, 20 bp, synthetic construct
  misc_feature 1..20: note = Synthetic Construct
  source 1..20: mol_type = other DNA
aggcatgagtctgcatctta

SEQ ID NO: 233 - DNA, 20 bp, synthetic construct
  misc_feature 1..20: note = Synthetic Construct
  source 1..20: mol_type = other DNA
ggcctgactgtggtgctcat

SEQ ID NO: 234 - DNA, 20 bp, synthetic construct
  misc_feature 1..20: note = Synthetic Construct
  source 1..20: mol_type = other DNA
agattccagctgctggtaca

SEQ ID NO: 235 - DNA, 20 bp, synthetic construct
  misc_feature 1..20: note = Synthetic Construct
  source 1..20: mol_type = other DNA
gtacatgggagctggcaacc

SEQ ID NO: 236 - DNA, 20 bp, synthetic construct
  misc_feature 1..20: note = Synthetic Construct
  source 1..20: mol_type = other DNA
cacttgcttacaaatgtcta

SEQ ID NO: 237 - DNA, 20 bp, synthetic construct
  misc_feature 1..20: note = Synthetic Construct
  source 1..20: mol_type = other DNA
agctgctggtacatgggagc

SEQ ID NO: 238 - DNA, 20 bp, synthetic construct
  misc_feature 1..20: note = Synthetic Construct
  source 1..20: mol_type = other DNA
agcaccacagtcaggccttg

SEQ ID NO: 239 - DNA, 20 bp, synthetic construct
  misc_feature 1..20: note = Synthetic Construct
  source 1..20: mol_type = other DNA
ctaaggtccccactgcctgc

SEQ ID NO: 240 - DNA, 20 bp, synthetic construct
  misc_feature 1..20: note = Synthetic Construct
  source 1..20: mol_type = other DNA
ttttttctccagcaggcagt

SEQ ID NO: 241 - DNA, 20 bp, synthetic construct
  misc_feature 1..20: note = Synthetic Construct
  source 1..20: mol_type = other DNA
aggcctgactgtggtgctca

SEQ ID NO: 242 - DNA, 20 bp, synthetic construct
  misc_feature 1..20: note = Synthetic Construct
  source 1..20: mol_type = other DNA
agctcccatgtaccagcagc

SEQ ID NO: 243 - DNA, 20 bp, synthetic construct
  misc_feature 1..20: note = Synthetic Construct
  source 1..20: mol_type = other DNA
ggcaacccggttttgagaca

SEQ ID NO: 244 - DNA, 20 bp, synthetic construct
  misc_feature 1..20: note = Synthetic Construct
  source 1..20: mol_type = other DNA
tttttctccagcaggcagtg

SEQ ID NO: 245 - DNA, 20 bp, synthetic construct
  misc_feature 1..20: note = Synthetic Construct
  source 1..20: mol_type = other DNA
tggcaacccggttttgagac

SEQ ID NO: 246 - DNA, 20 bp, synthetic construct
  misc_feature 1..20: note = Synthetic Construct
  source 1..20: mol_type = other DNA
caacagccctgtctcaaaac

SEQ ID NO: 247 - DNA, 20 bp, synthetic construct
  misc_feature 1..20: note = Synthetic Construct
  source 1..20: mol_type = other DNA
gcaccacagtcaggccttga

SEQ ID NO: 248 - DNA, 20 bp, synthetic construct
  misc_feature 1..20: note = Synthetic Construct
  source 1..20: mol_type = other DNA
aacagccctgtctcaaaacc

SEQ ID NO: 249 - DNA, 20 bp, synthetic construct
  misc_feature 1..20: note = Synthetic Construct
  source 1..20: mol_type = other DNA
gattccagctgctggtacat

SEQ ID NO: 250 - DNA, 20 bp, synthetic construct
  misc_feature 1..20: note = Synthetic Construct
  source 1..20: mol_type = other DNA
tgccttcagattccagctgc

SEQ ID NO: 251 - DNA, 20 bp, synthetic construct
  misc_feature 1..20: note = Synthetic Construct
  source 1..20: mol_type = other DNA
taccagcagctggaatctga

SEQ ID NO: 252 - DNA, 21 bp, synthetic construct
  misc_feature 1..21: note = Synthetic Construct
  source 1..21: mol_type = other DNA
taccttgcatgaggcccagtg

SEQ ID NO: 253 - DNA, 21 bp, synthetic construct
  misc_feature 1..21: note = Synthetic Construct
  source 1..21: mol_type = other DNA
catgttgaccacatcatggga

SEQ ID NO: 254 - DNA, 21 bp, synthetic construct
  misc_feature 1..21: note = Synthetic Construct
  source 1..21: mol_type = other DNA
gcgccctgcccatgcagttta

SEQ ID NO: 255 - DNA, 21 bp, synthetic construct
  misc_feature 1..21: note = Synthetic Construct
  source 1..21: mol_type = other DNA
cagggttcttcttgctgcagg

SEQ ID NO: 256 - DNA, 21 bp, synthetic construct
  misc_feature 1..21: note = Synthetic Construct
  source 1..21: mol_type = other DNA
tttccagggttcttcttgctg

SEQ ID NO: 257 - DNA, 21 bp, synthetic construct
  misc_feature 1..21: note = Synthetic Construct
  source 1..21: mol_type = other DNA
gagaccccatcatggtgctct

SEQ ID NO: 258 - DNA, 21 bp, synthetic construct
  misc_feature 1..21: note = Synthetic Construct
  source 1..21: mol_type = other DNA
aggacactttgcacctcattg

SEQ ID NO: 259 - DNA, 21 bp, synthetic construct
  misc_feature 1..21: note = Synthetic Construct
  source 1..21: mol_type = other DNA
cacctcattggagagcaccat

SEQ ID NO: 260 - DNA, 21 bp, synthetic construct
  misc_feature 1..21: note = Synthetic Construct
  source 1..21: mol_type = other DNA
accaggcgacctgggtgggcc

SEQ ID NO: 261 - DNA, 21 bp, synthetic construct
  misc_feature 1..21: note = Synthetic Construct
  source 1..21: mol_type = other DNA
agcacttccttctgcacagag

SEQ ID NO: 262 - DNA, 21 bp, synthetic construct
  misc_feature 1..21: note = Synthetic Construct
  source 1..21: mol_type = other DNA
accactcgtatggagagtcac

SEQ ID NO: 263 - DNA, 21 bp, synthetic construct
  misc_feature 1..21: note = Synthetic Construct
  source 1..21: mol_type = other DNA
cgtgactctccatacgagtgg

SEQ ID NO: 264 - DNA, 21 bp, synthetic construct
  misc_feature 1..21: note = Synthetic Construct
  source 1..21: mol_type = other DNA
ctctgggccctgccacccacg

SEQ ID NO: 265 - DNA, 21 bp, synthetic construct
  misc_feature 1..21: note = Synthetic Construct
  source 1..21: mol_type = other DNA
agagcttggttcagtgggtga

SEQ ID NO: 266 - DNA, 21 bp, synthetic construct
  misc_feature 1..21: note = Synthetic Construct
  source 1..21: mol_type = other DNA
gagcttggttcagtgggtgaa

SEQ ID NO: 267 - DNA, 21 bp, synthetic construct
  misc_feature 1..21: note = Synthetic Construct
  source 1..21: mol_type = other DNA
gttggagcaccagcgatgaag

SEQ ID NO: 268 - DNA, 21 bp, synthetic construct
  misc_feature 1..21: note = Synthetic Construct
  source 1..21: mol_type = other DNA
ttggagcaccagcgatgaagg

SEQ ID NO: 269 - DNA, 21 bp, synthetic construct
  misc_feature 1..21: note = Synthetic Construct
  source 1..21: mol_type = other DNA
tgttggagcaccagcgatgaa

SEQ ID NO: 270 - DNA, 21 bp, synthetic construct
  misc_feature 1..21: note = Synthetic Construct
  source 1..21: mol_type = other DNA
tttgttggagcaccagcgatg

SEQ ID NO: 271 - DNA, 21 bp, synthetic construct
  misc_feature 1..21: note = Synthetic Construct
  source 1..21: mol_type = other DNA
ttgttggagcaccagcgatga

SEQ ID NO: 272 - DNA, 21 bp, synthetic construct
  misc_feature 1..21: note = Synthetic Construct
  source 1..21: mol_type = other DNA
tccttcatcgctggtgctcca

SEQ ID NO: 273 - DNA, 21 bp, synthetic construct
  misc_feature 1..21: note = Synthetic Construct
  source 1..21: mol_type = other DNA
gaagttccgtgtacacgatga

SEQ ID NO: 274 - DNA, 21 bp, synthetic construct
  misc_feature 1..21: note = Synthetic Construct
  source 1..21: mol_type = other DNA
gatctggactcagcatttgga

SEQ ID NO: 275 - DNA, 21 bp, synthetic construct
  misc_feature 1..21: note = Synthetic Construct
  source 1..21: mol_type = other DNA
tctgtgtgaaaacgcagtgat

SEQ ID NO: 276 - DNA, 21 bp, synthetic construct
  misc_feature 1..21: note = Synthetic Construct
  source 1..21: mol_type = other DNA
ctctgtgtgaaaacgcagtga

SEQ ID NO: 277 - DNA, 21 bp, synthetic construct
  misc_feature 1..21: note = Synthetic Construct
  source 1..21: mol_type = other DNA
tctctgtgtgaaaacgcagtg

SEQ ID NO: 278 - DNA, 21 bp, synthetic construct
  misc_feature 1..21: note = Synthetic Construct
  source 1..21: mol_type = other DNA
acacagagaaaaatcactcgc

SEQ ID NO: 279 - DNA, 21 bp, synthetic construct
  misc_feature 1..21: note = Synthetic Construct
  source 1..21: mol_type = other DNA
cacacagagaaaaatcactcg

SEQ ID NO: 280 - DNA, 21 bp, synthetic construct
  misc_feature 1..21: note = Synthetic Construct
  source 1..21: mol_type = other DNA
acttgacccctgcccacctct

SEQ ID NO: 281 - DNA, 21 bp, synthetic construct
  misc_feature 1..21: note = Synthetic Construct
  source 1..21: mol_type = other DNA
ctccagcaggcagtggggacc

SEQ ID NO: 282 - DNA, 21 bp, synthetic construct
  misc_feature 1..21: note = Synthetic Construct
  source 1..21: mol_type = other DNA
gaattgtgagttcctcagtgt

SEQ ID NO: 283 - DNA, 21 bp, synthetic construct
  misc_feature 1..21: note = Synthetic Construct
  source 1..21: mol_type = other DNA
actgctgacagaagactgaaa

SEQ ID NO: 284 - DNA, 21 bp, synthetic construct
  misc_feature 1..21: note = Synthetic Construct
  source 1..21: mol_type = other DNA
tccagcaggcagtggggacct

SEQ ID NO: 285 - DNA, 21 bp, synthetic construct
  misc_feature 1..21: note = Synthetic Construct
  source 1..21: mol_type = other DNA
agtcttctgtcagcagtaaaa

SEQ ID NO: 286 - DNA, 21 bp, synthetic construct
  misc_feature 1..21: note = Synthetic Construct
  source 1..21: mol_type = other DNA
ctgctgacagaagactgaaag

SEQ ID NO: 287 - DNA, 21 bp, synthetic construct
  misc_feature 1..21: note = Synthetic Construct
  source 1..21: mol_type = other DNA
taagcaagtgagaggcacatt

SEQ ID NO: 288 - DNA, 21 bp, synthetic construct
  misc_feature 1..21: note = Synthetic Construct
  source 1..21: mol_type = other DNA
aaggatggaaaactacattga

SEQ ID NO: 289 - DNA, 21 bp, synthetic construct
  misc_feature 1..21: note = Synthetic Construct
  source 1..21: mol_type = other DNA
tttgaaggatggaaaactaca

SEQ ID NO: 290 - DNA, 21 bp, synthetic construct
  misc_feature 1..21: note = Synthetic Construct
  source 1..21: mol_type = other DNA
ccatccttcaaataaacatgt

SEQ ID NO: 291 - DNA, 21 bp, synthetic construct
  misc_feature 1..21: note = Synthetic Construct
  source 1..21: mol_type = other DNA
catccttcaaataaacatgtc

SEQ ID NO: 292 - DNA, 21 bp, synthetic construct
  misc_feature 1..21: note = Synthetic Construct
  source 1..21: mol_type = other DNA
ttaaaataacttcaatgtagt

SEQ ID NO: 293 - DNA, 21 bp, synthetic construct
  misc_feature 1..21: note = Synthetic Construct
  source 1..21: mol_type = other DNA
aaataacttcaatgtagtttt

SEQ ID NO: 294 - DNA, 21 bp, synthetic construct
  misc_feature 1..21: note = Synthetic Construct
  source 1..21: mol_type = other DNA
taaaataacttcaatgtagtt

SEQ ID NO: 295 - DNA, 21 bp, synthetic construct
  misc_feature 1..21: note = Synthetic Construct
  source 1..21: mol_type = other DNA
aaaataacttcaatgtagttt

SEQ ID NO: 296 - DNA, 21 bp, synthetic construct
  misc_feature 1..21: note = Synthetic Construct
  source 1..21: mol_type = other DNA
aaaaaatatataagttttact

SEQ ID NO: 297 - DNA, 21 bp, synthetic construct
  misc_feature 1..21: note = Synthetic Construct
  source 1..21: mol_type = other DNA
ttctccagcaggcagtgggga

SEQ ID NO: 298 - DNA, 21 bp, synthetic construct
  misc_feature 1..21: note = Synthetic Construct
  source 1..21: mol_type = other DNA
aaaaatatataagttttactg

SEQ ID NO: 299 - DNA, 21 bp, synthetic construct
  misc_feature 1..21: note = Synthetic Construct
  source 1..21: mol_type = other DNA
tggtgtgaggaagagcgatgc

SEQ ID NO: 300 - DNA, 21 bp, synthetic construct
  misc_feature 1..21: note = Synthetic Construct
  source 1..21: mol_type = other DNA
cttagcccacagttctccatt

SEQ ID NO: 301 - DNA, 21 bp, synthetic construct
  misc_feature 1..21: note = Synthetic Construct
  source 1..21: mol_type = other DNA
tctccagcaggcagtggggac

SEQ ID NO: 302 - DNA, 21 bp, synthetic construct
  misc_feature 1..21: note = Synthetic Construct
  source 1..21: mol_type = other DNA
gagacagggctgttgtctccc

SEQ ID NO: 303 - DNA, 21 bp, synthetic construct
  misc_feature 1..21: note = Synthetic Construct
  source 1..21: mol_type = other DNA
agacagggctgttgtctccct

SEQ ID NO: 304 - DNA, 20 bp, synthetic construct
  source 1..20: mol_type = other DNA
taccttgcatgaggcccagt

SEQ ID NO: 305 - DNA, 20 bp, synthetic construct
  source 1..20: mol_type = other DNA
tggtcaacatgtaaactgca

SEQ ID NO: 306 - DNA, 20 bp, synthetic construct
  source 1..20: mol_type = other DNA
tgtgcgctgctgagctgagc

SEQ ID NO: 307 - DNA, 20 bp, synthetic construct
  source 1..20: mol_type = other DNA
ggtcaacatgtaaactgcat

SEQ ID NO: 308 - DNA, 20 bp, synthetic construct
  source 1..20: mol_type = other DNA
catgggagggtgacgtacgc

SEQ ID NO: 309 - DNA, 20 bp, synthetic construct
  source 1..20: mol_type = other DNA
atgttgaccacatcatggga

SEQ ID NO: 310 - DNA, 20 bp, synthetic construct
  source 1..20: mol_type = other DNA
tttacatgttgaccacatca

SEQ ID NO: 311 - DNA, 20 bp, synthetic construct
  source 1..20: mol_type = other DNA
acatgtaaactgcatgggca

SEQ ID NO: 312 - DNA, 20 bp, synthetic construct
  source 1..20: mol_type = other DNA
gcagcgcacaggatgttatt

SEQ ID NO: 313 - DNA, 20 bp, synthetic construct
  source 1..20: mol_type = other DNA
tgcgctgctgagctgagctg

SEQ ID NO: 314 - DNA, 20 bp, synthetic construct
  source 1..20: mol_type = other DNA
aacatgtaaactgcatgggc

SEQ ID NO: 315 - DNA, 20 bp, synthetic construct
  source 1..20: mol_type = other DNA
agctcagctcagcagcgcac

SEQ ID NO: 316 - DNA, 20 bp, synthetic construct
  source 1..20: mol_type = other DNA
ttacatgttgaccacatcat

SEQ ID NO: 317 - DNA, 20 bp, synthetic construct
  source 1..20: mol_type = other DNA
gtgcgctgctgagctgagct

SEQ ID NO: 318 - DNA, 20 bp, synthetic construct
  source 1..20: mol_type = other DNA
tacgtcaccctcccatgatg

SEQ ID NO: 319 - DNA, 20 bp, synthetic construct
  source 1..20: mol_type = other DNA
catgttgaccacatcatggg

SEQ ID NO: 320 - DNA, 20 bp, synthetic construct
  source 1..20: mol_type = other DNA
ggctctttctaccttgcatg

SEQ ID NO: 321 - DNA, 20 bp, synthetic construct
  source 1..20: mol_type = other DNA
ctaccttgcatgaggcccag

SEQ ID NO: 322 - DNA, 20 bp, synthetic construct
  source 1..20: mol_type = other DNA
cacccactgggcctcatgca

SEQ ID NO: 323 - DNA, 20 bp, synthetic construct
  source 1..20: mol_type = other DNA
gctgacgaccatgagcgaca

SEQ ID NO: 324 - DNA, 20 bp, synthetic construct
  source 1..20: mol_type = other DNA
gtgctgtctgtgcagacagg

SEQ ID NO: 325 - DNA, 20 bp, synthetic construct
  source 1..20: mol_type = other DNA
agacagcaccatgtcgctca

SEQ ID NO: 326 - DNA, 20 bp, synthetic construct
  source 1..20: mol_type = other DNA
atggtgctgtctgtgcagac

SEQ ID NO: 327 - DNA, 20 bp, synthetic construct
  source 1..20: mol_type = other DNA
gtcgtcagcatggtgtgtgt

SEQ ID NO: 328 - DNA, 20 bp, synthetic construct
  source 1..20: mol_type = other DNA
gtcgctcatggtcgtcagca

SEQ ID NO: 329 - DNA, 20 bp, synthetic construct
  source 1..20: mol_type = other DNA
cagggggcctggccacatga

SEQ ID NO: 330 - DNA, 20 bp, synthetic construct
  source 1..20: mol_type = other DNA
cagggttcttcttgctgcag

SEQ ID NO: 331 - DNA, 20 bp, synthetic construct
  source 1..20: mol_type = other DNA
tcttcttgctgcagggggcc

SEQ ID NO: 332 - DNA, 20 bp, synthetic construct
  source 1..20: mol_type = other DNA
agggttcttcttgctgcagg

SEQ ID NO: 333 - DNA, 20 bp, synthetic construct
  source 1..20: mol_type = other DNA
gcagggggcctggccacatg

SEQ ID NO: 334 - DNA, 20 bp, synthetic construct
  source 1..20: mol_type = other DNA
cctgcaaggtcttgcatcat

SEQ ID NO: 335 - DNA, 20 bp, synthetic construct
  source 1..20: mol_type = other DNA
cccatgatgcaagaccttgc

SEQ ID NO: 336 - DNA, 20 bp, synthetic construct
  source 1..20: mol_type = other DNA
ccctgcaaggtcttgcatca

SEQ ID NO: 337 - DNA, 20 bp, synthetic construct
  source 1..20: mol_type = other DNA
ccatgatgcaagaccttgca

SEQ ID NO: 338 - DNA, 20 bp, synthetic construct
  source 1..20: mol_type = other DNA
aaggacactttgcacctcat

SEQ ID NO: 339 - DNA, 20 bp, synthetic construct
  source 1..20: mol_type = other DNA
gtcttgcatcatgggaccga

SEQ ID NO: 340 - DNA, 20 bp, synthetic construct
  source 1..20: mol_type = other DNA
gcacagagaagggaagttta

SEQ ID NO: 341 - DNA, 20 bp, synthetic construct
  source 1..20: mol_type = other DNA
cctctggacatcgtcatcac

SEQ ID NO: 342 - DNA, 20 bp, synthetic construct
  source 1..20: mol_type = other DNA
ctgtgatgacgatgtccaga

SEQ ID NO: 343 - DNA, 20 bp, synthetic construct
  source 1..20: mol_type = other DNA
catcatggtgctctccaatg

SEQ ID NO: 344 - DNA, 20 bp, synthetic construct
  source 1..20: mol_type = other DNA
cctgtgatgacgatgtccag

SEQ ID NO: 345 - DNA, 20 bp, synthetic construct
  source 1..20: mol_type = other DNA
tgcaatgttggtcagatgtc

SEQ ID NO: 346 - DNA, 20 bp, synthetic construct
  source 1..20: mol_type = other DNA
tcatcacaggtgagagtgtc

SEQ ID NO: 347 - DNA, 20 bp, synthetic construct
  source 1..20: mol_type = other DNA
tcattggagagcaccatgat

SEQ ID NO: 348 - DNA, 20 bp, synthetic construct
  source 1..20: mol_type = other DNA
ctcattggagagcaccatga

SEQ ID NO: 349 - DNA, 20 bp, synthetic construct
  source 1..20: mol_type = other DNA
gcaccatgatggggtctcca

SEQ ID NO: 350 - DNA, 20 bp, synthetic construct
  source 1..20: mol_type = other DNA
tgtctcttctgatttcacca

SEQ ID NO: 351 - DNA, 20 bp, synthetic construct
  source 1..20: mol_type = other DNA
cattggagagcaccatgatg

SEQ ID NO: 352 - DNA, 20 bp, synthetic construct
  source 1..20: mol_type = other DNA
gtctcttctgatttcaccag

SEQ ID NO: 353 - DNA, 20 bp, synthetic construct
  source 1..20: mol_type = other DNA
acttccttctgcacagagaa

SEQ ID NO: 354 - DNA, 20 bp, synthetic construct
  source 1..20: mol_type = other DNA
acttcccttctctgtgcaga

SEQ ID NO: 355 - DNA, 20 bp, synthetic construct
  source 1..20: mol_type = other DNA
gacgatgtccagagggtcac

SEQ ID NO: 356 - DNA, 20 bp, synthetic construct
  source 1..20: mol_type = other DNA
agcatctgtaggtccctgca

SEQ ID NO: 357 - DNA, 20 bp, synthetic construct
  source 1..20: mol_type = other DNA
catgggaccgatggagaagt

SEQ ID NO: 358 - DNA, 20 bp, synthetic construct
  source 1..20: mol_type = other DNA
gggagctgacaactgatagg

SEQ ID NO: 359 - DNA, 20 bp, synthetic construct
  source 1..20: mol_type = other DNA
ctgtctcttctgatttcacc

SEQ ID NO: 360 - DNA, 20 bp, synthetic construct
  source 1..20: mol_type = other DNA
tctgatttcaccaggggacc

SEQ ID NO: 361 - DNA, 20 bp, synthetic construct
  source 1..20: mol_type = other DNA
ctgggagctgacaactgata

SEQ ID NO: 362 - DNA, 20 bp, synthetic construct
  source 1..20: mol_type = other DNA
tgggagctgacaactgatag

SEQ ID NO: 363 - DNA, 20 bp, synthetic construct
  source 1..20: mol_type = other DNA
ctgatttcaccaggggacct

SEQ ID NO: 364 - DNA, 20 bp, synthetic construct
  source 1..20: mol_type = other DNA
tttcaccaggggacctgggt

SEQ ID NO: 365 - DNA, 20 bp, synthetic construct
  source 1..20: mol_type = other DNA
aacagaaccgtagcatctgt

SEQ ID NO: 366 - DNA, 20 bp, synthetic construct
  source 1..20: mol_type = other DNA
atttcaccaggggacctggg

SEQ ID NO: 367 - DNA, 20 bp, synthetic construct
  source 1..20: mol_type = other DNA
aaggttttctgtggactcct

SEQ ID NO: 368 - DNA, 20 bp, synthetic construct
  source 1..20: mol_type = other DNA
actgggagctgacaactgat

SEQ ID NO: 369 - DNA, 20 bp, synthetic construct
  source 1..20: mol_type = other DNA
tggccttggagaccccatca

SEQ ID NO: 370 - DNA, 20 bp, synthetic construct
  source 1..20: mol_type = other DNA
cctggcccacccaggtcccc

SEQ ID NO: 371 - DNA, 20 bp, synthetic construct
  source 1..20: mol_type = other DNA
accgatggagaagttggcct

SEQ ID NO: 372 - DNA, 20 bp, synthetic construct
  source 1..20: mol_type = other DNA
gtcagctcccagtgaccctc

SEQ ID NO: 373 - DNA, 20 bp, synthetic construct
  source 1..20: mol_type = other DNA
acgatgtccagagggtcact

SEQ ID NO: 374 - DNA, 20 bp, synthetic construct
  source 1..20: mol_type = other DNA
tccaaggccaacttctccat

SEQ ID NO: 375 - DNA, 20 bp, synthetic construct
  source 1..20: mol_type = other DNA
ccaggggacctgggtgggcc

SEQ ID NO: 376 - DNA, 20 bp, synthetic construct
  source 1..20: mol_type = other DNA
tgacatctgaccaacattgc

SEQ ID NO: 377 - DNA, 20 bp, synthetic construct
  source 1..20: mol_type = other DNA
gcagggacctacagatgcta

SEQ ID NO: 378 - DNA, 20 bp, synthetic construct
  source 1..20: mol_type = other DNA
agacagtcatcctgcaatgt

SEQ ID NO: 379 - DNA, 20 bp, synthetic construct
  source 1..20: mol_type = other DNA
cacttccttctgcacagaga

SEQ ID NO: 380 - DNA, 20 bp, synthetic construct
  source 1..20: mol_type = other DNA
ccacagaaaaccttccctcc

SEQ ID NO: 381 - DNA, 20 bp, synthetic construct
  source 1..20: mol_type = other DNA
ccttccctcctggcccaccc

SEQ ID NO: 382 - DNA, 20 bp, synthetic construct
  source 1..20: mol_type = other DNA
ccaggagggaaggttttctg

SEQ ID NO: 383 - DNA, 20 bp, synthetic construct
  source 1..20: mol_type = other DNA
gggacctgggtgggccagga

SEQ ID NO: 384 - DNA, 20 bp, synthetic construct
  source 1..20: mol_type = other DNA
cctgggtgggccaggaggga

SEQ ID NO: 385 - DNA, 20 bp, synthetic construct
  source 1..20: mol_type = other DNA
ggggacctgggtgggccagg

SEQ ID NO: 386 - DNA, 20 bp, synthetic construct
  source 1..20: mol_type = other DNA
tgcagagaacctacgttcat

SEQ ID NO: 387 - DNA, 20 bp, synthetic construct
  source 1..20: mol_type = other DNA
catgaacgtaggttctctgc

SEQ ID NO: 388 - DNA, 20 bp, synthetic construct
  source 1..20: mol_type = other DNA
ctgcagagaacctacgttca

SEQ ID NO: 389 - DNA, 20 bp, synthetic construct
  source 1..20: mol_type = other DNA
tccgtgactctccatacgag

SEQ ID NO: 390 - DNA, 20 bp, synthetic construct
  source 1..20: mol_type = other DNA
atgaacgtaggttctctgca

SEQ ID NO: 391 - DNA, 20 bp, synthetic construct
  source 1..20: mol_type = other DNA
gctctctcctgccagaaccg

SEQ ID NO: 392 - DNA, 20 bp, synthetic construct
  source 1..20: mol_type = other DNA
agcatctgtaggttcctccg

SEQ ID NO: 393 - DNA, 20 bp, synthetic construct
  source 1..20: mol_type = other DNA
taggttctctgcagggccca

SEQ ID NO: 394 - DNA, 20 bp, synthetic construct
  source 1..20: mol_type = other DNA
ctcgagtttgaccactcgta

SEQ ID NO: 395 - DNA, 20 bp, synthetic construct
  source 1..20: mol_type = other DNA
accactcgtatggagagtca

SEQ ID NO: 396 - DNA, 20 bp, synthetic construct
  source 1..20: mol_type = other DNA
ctctctcctgccagaaccgt

SEQ ID NO: 397 - DNA, 20 bp, synthetic construct
  source 1..20: mol_type = other DNA
ggcccagaggaaagtcggcc

SEQ ID NO: 398 - DNA, 20 bp, synthetic construct
  source 1..20: mol_type = other DNA
ctggaatgttccgttgacct

SEQ ID NO: 399 - DNA, 20 bp, synthetic construct
  source 1..20: mol_type = other DNA
tggaatgttccgttgacctt

SEQ ID NO: 400 - DNA, 20 bp, synthetic construct
  source 1..20: mol_type = other DNA
tctgcagggcccaaggtcaa

SEQ ID NO: 401 - DNA, 20 bp, synthetic construct
  source 1..20: mol_type = other DNA
gcatctgtaggttcctccgt

SEQ ID NO: 402 - DNA, 20 bp, synthetic construct
  source 1..20: mol_type = other DNA
ggatagatggtacatgtcat

SEQ ID NO: 403 - DNA, 20 bp, synthetic construct
  source 1..20: mol_type = other DNA
caaggtcaacggaacattcc

SEQ ID NO: 404 - DNA, 20 bp, synthetic construct
  source 1..20: mol_type = other DNA
ccgggccccacggttctggc

SEQ ID NO: 405 - DNA, 20 bp, synthetic construct
  source 1..20: mol_type = other DNA
atgacatgtaccatctatcc

SEQ ID NO: 406 - DNA, 20 bp, synthetic construct
  source 1..20: mol_type = other DNA
tctctcctgccagaaccgtg

SEQ ID NO: 407 - DNA, 20 bp, synthetic construct
  source 1..20: mol_type = other DNA
ttcatgggcctccccctccc

SEQ ID NO: 408 - DNA, 20 bp, synthetic construct
  source 1..20: mol_type = other DNA
tgtaccatctatccagggag

SEQ ID NO: 409 - DNA, 20 bp, synthetic construct
  source 1..20: mol_type = other DNA
agggggaggcccatgaacgt

SEQ ID NO: 410 - DNA, 20 bp, synthetic construct
  source 1..20: mol_type = other DNA
ataggagctccgggagctgc

SEQ ID NO: 411 - DNA, 20 bp, synthetic construct
  source 1..20: mol_type = other DNA
attccaggccgactttcctc

SEQ ID NO: 412 - DNA, 20 bp, synthetic construct
  source 1..20: mol_type = other DNA
taggttcctccgtgggtggc

SEQ ID NO: 413 - DNA, 20 bp, synthetic construct
  source 1..20: mol_type = other DNA
ttccaggccgactttcctct

SEQ ID NO: 414 - DNA, 20 bp, synthetic construct
  source 1..20: mol_type = other DNA
atgtaccatctatccaggga

SEQ ID NO: 415 - DNA, 20 bp, synthetic construct
  source 1..20: mol_type = other DNA
tgacatgtaccatctatcca

SEQ ID NO: 416 - DNA, 20 bp, synthetic construct
  source 1..20: mol_type = other DNA
ccagaaccgtggggcccggc

SEQ ID NO: 417 - DNA, 20 bp, synthetic construct
  source 1..20: mol_type = other DNA
cctgccagaaccgtggggcc

SEQ ID NO: 418 - DNA, 20 bp, synthetic construct
  source 1..20: mol_type = other DNA
cagaaccgtggggcccggct

SEQ ID NO: 419 - DNA, 20 bp, synthetic construct
  source 1..20: mol_type = other DNA
ggaggaacctacagatgctt

SEQ ID NO: 420 - DNA, 20 bp, synthetic construct
  source 1..20: mol_type = other DNA
ccagccgggccccacggttc

SEQ ID NO: 421 - DNA, 20 bp, synthetic construct
  source 1..20: mol_type = other DNA
gtaccatctatccagggagg

SEQ ID NO: 422 - DNA, 20 bp, synthetic construct
  source 1..20: mol_type = other DNA
gtacatgtcataggagctcc

SEQ ID NO: 423 - DNA, 20 bp, synthetic construct
  source 1..20: mol_type = other DNA
catgtaccatctatccaggg

SEQ ID NO: 424 - DNA, 20 bp, synthetic construct
  source 1..20: mol_type = other DNA
ccatctatccagggaggggg

SEQ ID NO: 425 - DNA, 20 bp, synthetic construct
  source 1..20: mol_type = other DNA
ggtacatgtcataggagctc

SEQ ID NO: 426 - DNA, 20 bp, synthetic construct
  source 1..20: mol_type = other DNA
ggcagggcccagaggaaagt

SEQ ID NO: 427 - DNA, 20 bp, synthetic construct
  source 1..20: mol_type = other DNA
cctccccctccctggataga

SEQ ID NO: 428 - DNA, 20 bp, synthetic construct
  source 1..20: mol_type = other DNA
tctgtaggttcctccgtggg

SEQ ID NO: 429 - DNA, 20 bp, synthetic construct
  source 1..20: mol_type = other DNA
ctgtgacagaaacaagcagt

SEQ ID NO: 430 - DNA, 20 bp, synthetic construct
  source 1..20: mol_type = other DNA
gaaggtttctcatatagacc

SEQ ID NO: 431 - DNA, 20 bp, synthetic construct
  source 1..20: mol_type = other DNA
gctccgggagctgcaggaca

SEQ ID NO: 432 - DNA, 20 bp, synthetic construct
  source 1..20: mol_type = other DNA
gcttgtttctgtcacaggtg

SEQ ID NO: 433 - DNA, 20 bp, synthetic construct
  source 1..20: mol_type = other DNA
tgaccttgtcctgcagctcc

SEQ ID NO: 434 - DNA, 20 bp, synthetic construct
  source 1..20: mol_type = other DNA
aggttcctccgtgggtggca

SEQ ID NO: 435 - DNA, 20 bp, synthetic construct
  source 1..20: mol_type = other DNA
cctgtgacagaaacaagcag

SEQ ID NO: 436 - DNA, 20 bp, synthetic construct
  source 1..20: mol_type = other DNA
ctcagcccagccgggcccca

SEQ ID NO: 437 - DNA, 20 bp, synthetic construct
  source 1..20: mol_type = other DNA
ctgggccctgccacccacgg

SEQ ID NO: 438 - DNA, 20 bp, synthetic construct
  source 1..20: mol_type = other DNA
ccactgcttgtttctgtcac

SEQ ID NO: 439 - DNA, 20 bp, synthetic construct
  source 1..20: mol_type = other DNA
ccttctctctcagcccagcc

SEQ ID NO: 440 - DNA, 20 bp, synthetic construct
  source 1..20: mol_type = other DNA
gaaagagccgaagcatctgt

SEQ ID NO: 441 - DNA, 20 bp, synthetic construct
  source 1..20: mol_type = other DNA
accttctctctcagcccagc

SEQ ID NO: 442 - DNA, 20 bp, synthetic construct
  source 1..20: mol_type = other DNA
cccggctgggctgagagaga

SEQ ID NO: 443 - DNA, 20 bp, synthetic construct
  source 1..20: mol_type = other DNA
ccgtgggtggcagggcccag

SEQ ID NO: 444 - DNA, 20 bp, synthetic construct
  source 1..20: mol_type = other DNA
cctctgggccctgccaccca

SEQ ID NO: 445 - DNA, 20 bp, synthetic construct
  source 1..20: mol_type = other DNA
tactcaccggtttcggagct

SEQ ID NO: 446 - DNA, 20 bp, synthetic construct
  source 1..20: mol_type = other DNA
actgaaccaagctccgaaac

SEQ ID NO: 447 - DNA, 20 bp, synthetic construct
  source 1..20: mol_type = other DNA
ggtttcggagcttggttcag

SEQ ID NO: 448 - DNA, 20 bp, synthetic construct
  source 1..20: mol_type = other DNA
ggttctgtactcaccggttt

SEQ ID NO: 449 - DNA, 20 bp, synthetic construct
  source 1..20: mol_type = other DNA
gtttcggagcttggttcagt

SEQ ID NO: 450 - DNA, 20 bp, synthetic construct
  source 1..20: mol_type = other DNA
ggtgaaggccaactatttga

SEQ ID NO: 451 - DNA, 20 bp, synthetic construct
  source 1..20: mol_type = other DNA
taggaaacccttcaaatagt

SEQ ID NO: 452 - DNA, 20 bp, synthetic construct
  source 1..20: mol_type = other DNA
gtgaaggccaactatttgaa

SEQ ID NO: 453 - DNA, 20 bp, synthetic construct
  source 1..20: mol_type = other DNA
gagcttggttcagtgggtga

SEQ ID NO: 454 - DNA, 20 bp, synthetic construct
  source 1..20: mol_type = other DNA
aatcagaacatgcaggtgtc

SEQ ID NO: 455 - DNA, 20 bp, synthetic construct
  source 1..20: mol_type = other DNA
tgttgcagcaccagcgatga

SEQ ID NO: 456 - DNA, 20 bp, synthetic construct
  source 1..20: mol_type = other DNA
atcagaacatgcaggtgtct

SEQ ID NO: 457 - DNA, 20 bp, synthetic construct
  source 1..20: mol_type = other DNA
tcagaacatgcaggtgtctg

SEQ ID NO: 458 - DNA, 20 bp, synthetic construct
  source 1..20: mol_type = other DNA
aggtcccaatcagaacatgc

SEQ ID NO: 459 - DNA, 20 bp, synthetic construct
  source 1..20: mol_type = other DNA
ctggggttacctggaagaag

SEQ ID NO: 460 - DNA, 20 bp, synthetic construct
  source 1..20: mol_type = other DNA
gacacctgcatgttctgatt

SEQ ID NO: 461 - DNA, 20 bp, synthetic construct
  source 1..20: mol_type = other DNA
tgcaggtgtctggggttacc

SEQ ID NO: 462 - DNA, 20 bp, synthetic construct
  source 1..20: mol_type = other DNA
agacacctgcatgttctgat

SEQ ID NO: 463 - DNA, 20 bp, synthetic construct
  source 1..20: mol_type = other DNA
gaagaggatgatgaccactg

SEQ ID NO: 464 - DNA, 20 bp, synthetic construct
  source 1..20: mol_type = other DNA
tgttctgattgggacctcag

SEQ ID NO: 465 - DNA, 20 bp, synthetic construct
  source 1..20: mol_type = other DNA
tcttctttctccttcatcgc

SEQ ID NO: 466 - DNA, 20 bp, synthetic construct
  source 1..20: mol_type = other DNA
agcgatgaaggagaaagaag

SEQ ID NO: 467 - DNA, 20 bp, synthetic construct
  source 1..20: mol_type = other DNA
gatgaaggagaaagaagagg

SEQ ID NO: 468 - DNA, 20 bp, synthetic construct
  source 1..20: mol_type = other DNA
agaagaggaggaggatgaag

SEQ ID NO: 469 - DNA, 20 bp, synthetic construct
  source 1..20: mol_type = other DNA
gaaggagaaagaagaggagg

SEQ ID NO: 470 - DNA, 20 bp, synthetic construct
  source 1..20: mol_type = other DNA
cagggaacagaacagtgaac

SEQ ID NO: 471 - DNA, 20 bp, synthetic construct
  source 1..20: mol_type = other DNA
ttctacagatgctgttgtaa

SEQ ID NO: 472 - DNA, 20 bp, synthetic construct
  source 1..20: mol_type = other DNA
agggaacagaacagtgaaca

SEQ ID NO: 473 - DNA, 20 bp, synthetic construct
  source 1..20: mol_type = other DNA
gaacagaacagtgaacaggg

SEQ ID NO: 474 - DNA, 20 bp, synthetic construct
  source 1..20: mol_type = other DNA
agaacagtgaacagggaggt

SEQ ID NO: 475 - DNA, 20 bp, synthetic construct
  source 1..20: mol_type = other DNA
ttcactgttctgttccctgc

SEQ ID NO: 476 - DNA, 20 bp, synthetic construct
  source 1..20: mol_type = other DNA
taatggaccaagagcctgca

SEQ ID NO: 477 - DNA, 20 bp, synthetic construct
  source 1..20: mol_type = other DNA
ttctgttccctgcaggctct

SEQ ID NO: 478 - DNA, 20 bp, synthetic construct
  source 1..20: mol_type = other DNA
gtaatggaccaagagcctgc

SEQ ID NO: 479 - DNA, 20 bp, synthetic construct
  source 1..20: mol_type = other DNA
aacagatatcatcgtgtaca

SEQ ID NO: 480 - DNA, 20 bp, synthetic construct
  source 1..20: mol_type = other DNA
gtacacgatgatatctgttg

SEQ ID NO: 481 - DNA, 20 bp, synthetic construct
  source 1..20: mol_type = other DNA
gtgtacacgatgatatctgt

SEQ ID NO: 482 - DNA, 20 bp, synthetic construct
  source 1..20: mol_type = other DNA
tgtacacgatgatatctgtt

SEQ ID NO: 483 - DNA, 20 bp, synthetic construct
  source 1..20: mol_type = other DNA
tacacgatgatatctgttgg

SEQ ID NO: 484 - DNA, 20 bp, synthetic construct
  source 1..20: mol_type = other DNA
tgtgcatatgtcacctcctg

SEQ ID NO: 485 - DNA, 20 bp, synthetic construct
  source 1..20: mol_type = other DNA
gtgcatatgtcacctcctga

SEQ ID NO: 486 - DNA, 20 bp, synthetic construct
  source 1..20: mol_type = other DNA
atatctgttgggggtgtctt

SEQ ID NO: 487 - DNA, 20 bp, synthetic construct
  source 1..20: mol_type = other DNA
gatatctgttgggggtgtct

SEQ ID NO: 488 - DNA, 20 bp, synthetic construct
  source 1..20: mol_type = other DNA
ctctgatgaacaagaccctc

SEQ ID NO: 489 - DNA, 20 bp, synthetic construct
  source 1..20: mol_type = other DNA
aaatcactcgcccttctcag

SEQ ID NO: 490 - DNA, 20 bp, synthetic construct
  source 1..20: mol_type = other DNA
gtgtcttgggcctctgagaa

SEQ ID NO: 491 - DNA, 20 bp, synthetic construct
  source 1..20: mol_type = other DNA
tgatgaacaagaccctcagg

SEQ ID NO: 492 - DNA, 20 bp, synthetic construct
  source 1..20: mol_type = other DNA
ggtgtcttgggcctctgaga

SEQ ID NO: 493 - DNA, 20 bp, synthetic construct
  source 1..20: mol_type = other DNA
gtctaagtgccgtgttaaga

SEQ ID NO: 494 - DNA, 20 bp, synthetic construct
  source 1..20: mol_type = other DNA
tgtctaagtgccgtgttaag

SEQ ID NO: 495 - DNA, 20 bp, synthetic construct
  source 1..20: mol_type = other DNA
gagggagcctcttgtatgtt

SEQ ID NO: 496 - DNA, 20 bp, synthetic construct
  source 1..20: mol_type = other DNA
gcccacctctccaacctaac

SEQ ID NO: 497 - DNA, 20 bp, synthetic construct
  source 1..20: mol_type = other DNA
attgcagcctcaagtagact

SEQ ID NO: 498 - DNA, 20 bp, synthetic construct
  source 1..20: mol_type = other DNA
gactaggaagtaagccagtt

SEQ ID NO: 499 - DNA, 20 bp, synthetic construct
  source 1..20: mol_type = other DNA
aggaagtaagccagttaggt

SEQ ID NO: 500 - DNA, 20 bp, synthetic construct
  source 1..20: mol_type = other DNA
caagaggctccctcttaaca

SEQ ID NO: 501 - DNA, 20 bp, synthetic construct
  source 1..20: mol_type = other DNA
gtaagccagttaggttggag

SEQ ID NO: 502 - DNA, 20 bp, synthetic construct
  source 1..20: mol_type = other DNA
aggctgaaagctagtctgag

SEQ ID NO: 503 - DNA, 20 bp, synthetic construct
  source 1..20: mol_type = other DNA
cttacttcctagtctacttg

SEQ ID NO: 504 - DNA, 20 bp, synthetic construct
  source 1..20: mol_type = other DNA
gaaggctgaaagctagtctg

SEQ ID NO: 505 - DNA, 20 bp, synthetic construct
  source 1..20: mol_type = other DNA
aaggctgaaagctagtctga

SEQ ID NO: 506 - DNA, 20 bp, synthetic construct
  source 1..20: mol_type = other DNA
ctgaaagctagtctgagggg

SEQ ID NO: 507 - DNA, 20 bp, synthetic construct
  source 1..20: mol_type = other DNA
ttgaggctgcaatcacactg

SEQ ID NO: 508 - DNA, 20 bp, synthetic construct
  source 1..20: mol_type = other DNA
accttagacatttgtaagca

SEQ ID NO: 509 - DNA, 20 bp, synthetic construct
  source 1..20: mol_type = other DNA
gccagttaggttggagaggt

SEQ ID NO: 510 - DNA, 20 bp, synthetic construct
  source 1..20: mol_type = other DNA
tgggcaggggtcaagtgaaa

SEQ ID NO: 511 - DNA, 20 bp, synthetic construct
  source 1..20: mol_type = other DNA
cacaattccaaacatacaag

SEQ ID NO: 512 - DNA, 20 bp, synthetic construct
  source 1..20: mol_type = other DNA
ggggaggtggaacagcatga

SEQ ID NO: 513 - DNA, 20 bp, synthetic construct
  source 1..20: mol_type = other DNA
aggggaggtggaacagcatg

SEQ ID NO: 514 - DNA, 20 bp, synthetic construct
  source 1..20: mol_type = other DNA
gggcagacatgtttatttga

SEQ ID NO: 515 - DNA, 20 bp, synthetic construct
  source 1..20: mol_type = other DNA
agccagttaggttggagagg

SEQ ID NO: 516 - DNA, 20 bp, synthetic construct
  source 1..20: mol_type = other DNA
aaagctagtctgaggggagg

SEQ ID NO: 517 - DNA, 20 bp, synthetic construct
  source 1..20: mol_type = other DNA
caagtgaaatggagaattgt

SEQ ID NO: 518 - DNA, 20 bp, synthetic construct
  source 1..20: mol_type = other DNA
aggtggaacagcatgaggga

SEQ ID NO: 519 - DNA, 20 bp, synthetic construct
  source 1..20: mol_type = other DNA
cagacatgtttatttgaagg

SEQ ID NO: 520 - DNA, 20 bp, synthetic construct
  source 1..20: mol_type = other DNA
agacatgtttatttgaagga

SEQ ID NO: 521 - DNA, 20 bp, synthetic construct
  source 1..20: mol_type = other DNA
gttaggttggagaggtgggc

SEQ ID NO: 522 - DNA, 20 bp, synthetic construct
  source 1..20: mol_type = other DNA
tggaacagcatgagggaagg

SEQ ID NO: 523 - DNA, 20 bp, synthetic construct
  source 1..20: mol_type = other DNA
caggagacaactttggatca

SEQ ID NO: 524 - DNA, 20 bp, synthetic construct
  source 1..20: mol_type = other DNA
tcaagtgaaatggagaattg

SEQ ID NO: 525 - DNA, 20 bp, synthetic construct
  source 1..20: mol_type = other DNA
aataaacatgtctgccctca

SEQ ID NO: 526 - DNA, 20 bp, synthetic construct
  source 1..20: mol_type = other DNA
agctgctggtacattggagc

SEQ ID NO: 527 - DNA, 20 bp, synthetic construct
  source 1..20: mol_type = other DNA
taagttttactgctgacaga

SEQ ID NO: 528 - DNA, 20 bp, synthetic construct
  source 1..20: mol_type = other DNA
taggttggagaggtgggcag

SEQ ID NO: 529 - DNA, 20 bp, synthetic construct
  source 1..20: mol_type = other DNA
gagaggcacgttcagatttg

SEQ ID NO: 530 - DNA, 20 bp, synthetic construct
  source 1..20: mol_type = other DNA
ttaggttggagaggtgggca

SEQ ID NO: 531 - DNA, 20 bp, synthetic construct
  source 1..20: mol_type = other DNA
acgttcagatttgtggtgtg

SEQ ID NO: 532 - DNA, 20 bp, synthetic construct
  source 1..20: mol_type = other DNA
aacagccctgtctcaaaact

SEQ ID NO: 533 - DNA, 20 bp, synthetic construct
  source 1..20: mol_type = other DNA
gacatttgtaagcaagggag

SEQ ID NO: 534 - DNA, 20 bp, synthetic construct
  source 1..20: mol_type = other DNA
ggcaacccagttttgagaca

SEQ ID NO: 535 - DNA, 20 bp, synthetic construct
  source 1..20: mol_type = other DNA
ctccctagaagatcccctca

SEQ ID NO: 536 - DNA, 20 bp, synthetic construct
  source 1..20: mol_type = other DNA
ggccttgaggggatcttcta

SEQ ID NO: 537 - DNA, 20 bp, synthetic construct
  source 1..20: mol_type = other DNA
aaggcgtgagtctgcatctt

SEQ ID NO: 538 - DNA, 20 bp, synthetic construct
  source 1..20: mol_type = other DNA
caccacagtcaggccttgag

SEQ ID NO: 539 - DNA, 20 bp, synthetic construct
  source 1..20: mol_type = other DNA
atcccctcaaggcctgactg

SEQ ID NO: 540 - DNA, 20 bp, synthetic construct
  source 1..20: mol_type = other DNA
tggcaacccagttttgagac

SEQ ID NO: 541 - DNA, 20 bp, synthetic construct
  source 1..20: mol_type = other DNA
gagaattgtgggctaagcaa

SEQ ID NO: 542 - DNA, 20 bp, synthetic construct
  source 1..20: mol_type = other DNA
aggcgtgagtctgcatctta

SEQ ID NO: 543 - DNA, 20 bp, synthetic construct
  source 1..20: mol_type = other DNA
aggccttgaggggatcttct

SEQ ID NO: 544 - DNA, 20 bp, synthetic construct
  source 1..20: mol_type = other DNA
gcaggagacaactttggatc

SEQ ID NO: 545 - DNA, 20 bp, synthetic construct
  source 1..20: mol_type = other DNA
tcatgggcaggagacaactt

SEQ ID NO: 546 - DNA, 20 bp, synthetic construct
  source 1..20: mol_type = other DNA
agctccaatgtaccagcagc

SEQ ID NO: 547 - DNA, 20 bp, synthetic construct
  source 1..20: mol_type = other DNA
ggcctgactgtggtgctcat

SEQ ID NO: 548 - DNA, 20 bp, synthetic construct
  source 1..20: mol_type = other DNA
cccttgcttacaaatgtcta

SEQ ID NO: 549 - DNA, 20 bp, synthetic construct
  source 1..20: mol_type = other DNA
tgactgtggtgctcatgggc

SEQ ID NO: 550 - DNA, 20 bp, synthetic construct
  source 1..20: mol_type = other DNA
gagtgtgttttctctccagc

SEQ ID NO: 551 - DNA, 20 bp, synthetic construct
  source 1..20: mol_type = other DNA
agcaccacagtcaggccttg

SEQ ID NO: 552 - DNA, 20 bp, synthetic construct
  source 1..20: mol_type = other DNA
ctaaggtccccactgcctgc

SEQ ID NO: 553 - DNA, 20 bp, synthetic construct
  source 1..20: mol_type = other DNA
aggcctgactgtggtgctca

SEQ ID NO: 554 - DNA, 20 bp, synthetic construct
  source 1..20: mol_type = other DNA
tgcccatgagcaccacagtc

SEQ ID NO: 555 - DNA, 20 bp, synthetic construct
  source 1..20: mol_type = other DNA
caacagccctgtctcaaaac

SEQ ID NO: 556 - DNA, 20 bp, synthetic construct
  source 1..20: mol_type = other DNA
gcaccacagtcaggccttga

SEQ ID NO: 557 - DNA, 20 bp, synthetic construct
  source 1..20: mol_type = other DNA
ccttagacatttgtaagcaa

SEQ ID NO: 558 - DNA, 20 bp, synthetic construct
  source 1..20: mol_type = other DNA
ttttctctccagcaggcagt

SEQ ID NO: 559 - DNA, 20 bp, synthetic construct
  source 1..20: mol_type = other DNA
gattccagctgctggtacat

SEQ ID NO: 560 - DNA, 20 bp, synthetic construct
  source 1..20: mol_type = other DNA
tttctctccagcaggcagtg

SEQ ID NO: 561 - DNA, 20 bp, synthetic construct
  source 1..20: mol_type = other DNA
cgccttcagattccagctgc

SEQ ID NO: 562 - DNA, 20 bp, synthetic construct
  source 1..20: mol_type = other DNA
gttttctctccagcaggcag

SEQ ID NO: 563 - DNA, 20 bp, synthetic construct
  source 1..20: mol_type = other DNA
taccagcagctggaatctga

SEQ ID NO: 564 - DNA, 23 bp, synthetic construct
  source 1..23: mol_type = other DNA
gcgccctgcccatgcagtttaca

SEQ ID NO: 565 - DNA, 23 bp, synthetic construct
  source 1..23: mol_type = other DNA
catgttgaccacatcatgggagg

SEQ ID NO: 566 - DNA, 23 bp, synthetic construct
  source 1..23: mol_type = other DNA
taccttgcatgaggcccagtggg

SEQ ID NO: 567 - DNA, 23 bp, synthetic construct
  source 1..23: mol_type = other DNA
gagaaggactcaccctcatgtgg

SEQ ID NO: 568 - DNA, 23 bp, synthetic construct
  source 1..23: mol_type = other DNA
cagggttcttcttgctgcagggg

SEQ ID NO: 569 - DNA, 23 bp, synthetic construct
  source 1..23: mol_type = other DNA
tttccagggttcttcttgctgca

SEQ ID NO: 570 - DNA, 23 bp, synthetic construct
  source 1..23: mol_type = other DNA
aggacactttgcacctcattgga

SEQ ID NO: 571 - DNA, 23 bp, synthetic construct
  source 1..23: mol_type = other DNA
cacctcattggagagcaccatga

SEQ ID NO: 572 - DNA, 23 bp, synthetic construct
  source 1..23: mol_type = other DNA
accaggggacctgggtgggccag

SEQ ID NO: 573 - DNA, 23 bp, synthetic construct
  source 1..23: mol_type = other DNA
ctgtggactcctaggaagagagg

SEQ ID NO: 574 - DNA, 23 bp, synthetic construct
  source 1..23: mol_type = other DNA
tgtggactcctaggaagagaggt

SEQ ID NO: 575 - DNA, 23 bp, synthetic construct
  source 1..23: mol_type = other DNA
agcacttccttctgcacagagaa

SEQ ID NO: 576 - DNA, 23 bp, synthetic construct
  source 1..23: mol_type = other DNA
tcatatagacctggaggagaaga

SEQ ID NO: 577 - DNA, 23 bp, synthetic construct
  source 1..23: mol_type = other DNA
accactcgtatggagagtcacgg

SEQ ID NO: 578 - DNA, 23 bp, synthetic construct
  source 1..23: mol_type = other DNA
cgtgactctccatacgagtggtc

SEQ ID NO: 579 - DNA, 23 bp, synthetic construct
  source 1..23: mol_type = other DNA
tgtcacaggtgaggaaaccccat

SEQ ID NO: 580 - DNA, 23 bp, synthetic construct
  source 1..23: mol_type = other DNA
ctctgggccctgccacccacgga

SEQ ID NO: 581 - DNA, 23 bp, synthetic construct
  source 1..23: mol_type = other DNA
ctcacctgtgacagaaacaagca

SEQ ID NO: 582 - DNA, 23 bp, synthetic construct
  source 1..23: mol_type = other DNA
aagggtttcctagaacacgagac

SEQ ID NO: 583 - DNA, 23 bp, synthetic construct
  source 1..23: mol_type = other DNA
ctcacctctctcctgtctcgtgt

SEQ ID NO: 584 - DNA, 23 bp, synthetic construct
  source 1..23: mol_type = other DNA
ctagaacacgagacaggagagag

SEQ ID NO: 585 - DNA, 23 bp, synthetic construct
  source 1..23: mol_type = other DNA
ggagcttggttcagtgggtgaag

SEQ ID NO: 586 - DNA, 23 bp, synthetic construct
  source 1..23: mol_type = other DNA
tttgttgcagcaccagcgatgaa

SEQ ID NO: 587 - DNA, 23 bp, synthetic construct
  source 1..23: mol_type = other DNA
gttgcagcaccagcgatgaagga

SEQ ID NO: 588 - DNA, 23 bp, synthetic construct
  source 1..23: mol_type = other DNA
ttgcagcaccagcgatgaaggag

SEQ ID NO: 589 - DNA, 23 bp, synthetic construct
  source 1..23: mol_type = other DNA
tgttgcagcaccagcgatgaagg

SEQ ID NO: 590 - DNA, 23 bp, synthetic construct
  source 1..23: mol_type = other DNA
ttgttgcagcaccagcgatgaag

SEQ ID NO: 591 - DNA, 23 bp, synthetic construct
  source 1..23: mol_type = other DNA
tccttcatcgctggtgctgcaac

SEQ ID NO: 592 - DNA, 23 bp, synthetic construct
  source 1..23: mol_type = other DNA
gatcagggctcagcatttggaag

SEQ ID NO: 593 - DNA, 23 bp, synthetic construct
  source 1..23: mol_type = other DNA
gaagttccgtgtacacgatgata

SEQ ID NO: 594 - DNA, 23 bp, synthetic construct
  source 1..23: mol_type = other DNA
cctctctccaggactctgatgaa

SEQ ID NO: 595 - DNA, 23 bp, synthetic construct
  source 1..23: mol_type = other DNA
tctgtgtgaaaacgcagtgattc

SEQ ID NO: 596 - DNA, 23 bp, synthetic construct
  source 1..23: mol_type = other DNA
ctctgtgtgaaaacgcagtgatt

SEQ ID NO: 597 - DNA, 23 bp, synthetic construct
  source 1..23: mol_type = other DNA
tctctgtgtgaaaacgcagtgat

SEQ ID NO: 598 - DNA, 23 bp, synthetic construct
  source 1..23: mol_type = other DNA
acacagagaaaaatcactcgccc

SEQ ID NO: 599 - DNA, 23 bp, synthetic construct
  source 1..23: mol_type = other DNA
cacacagagaaaaatcactcgcc

SEQ ID NO: 600 - DNA, 23 bp, synthetic construct
  source 1..23: mol_type = other DNA
acttgacccctgcccacctctcc

SEQ ID NO: 601 - DNA, 23 bp, synthetic construct
  source 1..23: mol_type = other DNA
agccttctgtcagcagtaaaact

SEQ ID NO: 602 - DNA, 23 bp, synthetic construct
  source 1..23: mol_type = other DNA
gaattgtgagttcctcagtgtga

SEQ ID NO: 603 - DNA, 23 bp, synthetic construct
  source 1..23: mol_type = other DNA
ctgctgacagaaggctgaaagct

SEQ ID NO: 604 - DNA, 23 bp, synthetic construct
  source 1..23: mol_type = other DNA
taagcaagggagaggcacgttca

SEQ ID NO: 605 - DNA, 23 bp, synthetic construct
  source 1..23: mol_type = other DNA
aaggagggaaaactacattgaaa

SEQ ID NO: 606 - DNA, 23 bp, synthetic construct
  source 1..23: mol_type = other DNA
ccctccttcaaataaacatgtct

SEQ ID NO: 607 - DNA, 23 bp, synthetic construct
  source 1..23: mol_type = other DNA
tttgaaggagggaaaactacatt

SEQ ID NO: 608 - DNA, 23 bp, synthetic construct
  source 1..23: mol_type = other DNA
cctccttcaaataaacatgtctg

SEQ ID NO: 609 - DNA, 23 bp, synthetic construct
  source 1..23: mol_type = other DNA
aatgtagttttccctccttcaaa

SEQ ID NO: 610 - DNA, 23 bp, synthetic construct
  source 1..23: mol_type = other DNA
ttaaaataatttcaatgtagttt

SEQ ID NO: 611 - DNA, 23 bp, synthetic construct
  source 1..23: mol_type = other DNA
actgctgacagaaggctgaaagc

SEQ ID NO: 612 - DNA, 23 bp, synthetic construct
  source 1..23: mol_type = other DNA
aaaaaatatataagttttactgc

SEQ ID NO: 613 - DNA, 23 bp, synthetic construct
  source 1..23: mol_type = other DNA
aaataatttcaatgtagttttcc

SEQ ID NO: 614 - DNA, 23 bp, synthetic construct
  source 1..23: mol_type = other DNA
taaaataatttcaatgtagtttt

SEQ ID NO: 615 - DNA, 23 bp, synthetic construct
  source 1..23: mol_type = other DNA
gatcagggctcagcatttggaag

SEQ ID NO: 616 - DNA, 23 bp, synthetic construct
  source 1..23: mol_type = other DNA
aaaataatttcaatgtagttttc

SEQ ID NO: 617 - DNA, 23 bp, synthetic construct
  source 1..23: mol_type = other DNA
aaaaatatataagttttactgct

SEQ ID NO: 618 - DNA, 23 bp, synthetic construct
  source 1..23: mol_type = other DNA
tggtgtgaggaagagcgatgccc

SEQ ID NO: 619 - DNA, 23 bp, synthetic construct
  source 1..23: mol_type = other DNA
cttagcccacaattctccatttc

SEQ ID NO: 620 - DNA, 23 bp, synthetic construct
  source 1..23: mol_type = other DNA
ctctccagcaggcagtggggacc

SEQ ID NO: 621 - DNA, 23 bp, synthetic construct
  source 1..23: mol_type = other DNA
tctccagcaggcagtggggacct

SEQ ID NO: 622 - DNA, 23 bp, synthetic construct
  source 1..23: mol_type = other DNA
gagacagggctgttgtctcccta

SEQ ID NO: 623 - DNA, 23 bp, synthetic construct
  source 1..23: mol_type = other DNA
agacagggctgttgtctccctag

SEQ ID NO: 624 - DNA, 14666 bp, Homo sapiens
  source 1..14666: mol_type = other DNA
gagcacccactgggcctcatgcaaggtagaaagagcctgcgtacgtcaccctcccatgatgtggtcaacatgtaaactgcatgggcagggcgccaaataacatcctgtgcgctgctgagctgagctggggcgcggccgcctgtctgcacagacagcaccatgtcgctcatggtcgtcagcatggtgtgtgttggtgagtcctggaagggcatcgagggagggagtgcggggatggagatcggggcccagagttggagatataggcctggaagtggagttatgggcctagagatggagtgatgggcctagaagtggagatctgggcctggagtggagatctgggcctggagtggagatatgggcctggaggttgagatatgggcctgcagtagagatatgggcttgtagtggagacatgggcctggagatggagatatgggcctggagatggagatatgggcctgcagtagagataggggcctggagtggagatatgggcctggagtggagatatgggcctggagtggagatatgggcctggaggtggagatatgggcctggaggtggagatatgggcctggagtggagatatgggtctggaggtggagatacgggcctgcagtagagatatgggcctggagtggagatatgggccaggagtggagttatgggcctagaggtggatatctgggcctggagtggagatatgggcctaggaaggagatatgggcctgggtgtggagatatgggactggagaggtgatatgggcctggagtggagatatgggcttagggtggagatctgggcctggggcggagatatgggactggattggagataggggcctagggtggagatctgagcctggattggcgatatgggcctagggtggaaatatcagcctggagtggagatatgggcttggggtggggatatgggcctggaaactgggtctctgcacagccgacagccctgttcttgggtgcaggtaggcactgagggtgagtttaacttcagcccaggaagggcctggctgccaagactcacagcccagtgggggcagcaagggaggcctggtttgcctgcagatggatggtccatcatgatctttctttccagggttcttcttgctgcagggggcctggccacatgagggtgagtccttctccaaaccttcgggtgtcatctccccacataagaggattttcctgaaacaggagggaagtcctgtcggggagtctctcataaactaggaagagaggaccctggggtgctcagcccacatttctgacctcgcctccctggcctctcaaccccttggcagagtcaagttctgtggggaccagggttagactggggtgctcaaagctggggtgtgtggttgggaagtggtaggaacagcagatcctctgaggacaaaggtgttactcacacacttcagcgtttccatgatggtaggggctgcagtgtggctgctgtcattctaccagaagaggtgggaaaccacagccatggccctgacattccaaatcctctgatgggggctcagttgtttattttcgttcaggcatccgctgatatccattcacaaaggacatgccctccacctcatgtctaccctgtgttgttttatgtgagtaatcttacagtattaaaatctagtaggagtctctttactcagcacttgctcaaagttctcagctgaggcttttgttgtagggagacaccatgtctttgcgggatgggtccttccttcagccctgggcaccaaggtgtgatagtagccatagaaacgtggaaagcgaggagaatcttctgagcacagggagggaagggcagttccacatcctcctctctaaggcggcgcctccttctccccaaggtggtcaggacaagcccttgctgtctgcctggcccagccttgtggtgcctctaggacatgtcattcttcggtgtcactcttatcttgggtttaacaacttcagtctgtaaaaggaaggtggggtgcctgtccctgagctctacaacagaatattctggaacagccttttcatgggccctgtgacccccgcacacacagggacatacagatgtcggggttcacacacacactcccccagtgggtggtcagcacccagcaaccccctggtgatcgtggtcataggtcagagggctcctgtcttggattctccttgtcccacctcctgaatcccagagcttctgttgggcatgtccttgagggtcccatcacgcaggccctgactgtatttgtggtaaagggggattgaatacagggaaatgggtgctgtggtgggaagaataattgtccccagtgatgactacattctaatccctggagtctgtgactatttatgttataggggaagggactgaaggggaagatggagctcatggggagacagcctggactgtcccactgggctcagtgtaatcacaagggtgcacatgaaaggaggaggaagaggggagtggggattagagcagtccagtggaagtcttcaccagctttgaaggtggaggaaggccaagatccatgaatgcaggtggcctatagaggctggaaaagtcaaggaactgattctccagagtctccagagggaacaaagccctgcagatgccttgattttagcccaggaaaaatagggtccaatttctgtctccagtactggaaggtgtcagtgtggtctctcctgctgccatgcttctgataattttctacagcagcaacaggaaaccaacactggaacccaggtcaaggacaagttaagaaacaacccaaggaaagccaggcatggtggcaggtgcatgtaatcctagcgactcaggaggctgagggcaggagaatcacttgaacccaggagacagaggttgcagtgagcctagaccacaccacttcactccagcctgggtgaaggagtgagactctgtctccataattaattaattaattaaagaaaccaaacaaggagaaggttggctaccctgagatcagcaagggtgggatgatgatgccaccaccaggctccatccacatagggaggggttgatactcctccaaccagcaccaggagccagcctatggaagctggcaccatggagaaggcacaggcatggcaagagtggctcccagtccccaccaggaacagggtgtgtggacactggtgcctgccttattcatcagttcataccttctgccaaggattgcaattcatccaaaagagattgaaccaggctgataagagcctggatgtgcagcctatcctggttcctctttcacccccacataaacagcaggaaatacattagtgtgaaatagatacaacaccccaagagatgaggctcagcccagtgggaagggaatcagaggctactagagacagagggacagagaagagggagggagacagatggaaggacctgcaccaggagttaagggcacagaaaagaacatgaagacacagagaggaaggagagagacagacaccagcaaggggaagcctcactcattctaggtgccatggatgggatgataaagagagacaccttctaaactcacaacctctcttcctaggagtccacagaaaaccttccctcctggcccacccaggtcccctggtgaaatcagaagagacagtcatcctgcaatgttggtcagatgtcaggtttcagcacttccttctgcacagagaagggaagtttaaggacactttgcacctcattggagagcaccatgatggggtctccaaggccaacttctccatcggtcccatgatgcaagaccttgcagggacctacagatgctacggttctgttactcactccccctatcagttgtcagctcccagtgaccctctggacatcgtcatcacaggtgagagtgtccggacattctcattgtcattgggatgcagagtgaatgatccacgacttggaacccccaggtagttgtaaggaagatgagcttggtattcttatggagagagactgacttgctgaggtttgtaccaacagagacagagaaacaggagacacaagtacagaccaggtgtcataacagaggacagacacaggggccatacagggagttagaaaagacagaaagagttaaaagagacagacagacagacatgtcccagagagaggtgtccctccatgctgactttgctcacagacctggcacaggttagaagtttcatttctgttttacctccacaaagtgttctctaccaggagaacccaaggacacccatatttatgacctgagttgggccctgtggcctcaggccttgtggcacctacaggccatgtttattctgacacctctgccttccatgtaatggagagtaatcgtcccaggatatcatggccccagaacaccaacccctgtatgctgtgtgaacttgtggtctccagactggattctgtggctcacattccaaataaccccacatatgaaaggatcactgagaggcacagagaaaaatcaggaacaccaaaaagcaaagacataaacacacagagaatgagccagaggaaggagattgagagactcacagacacataaagagagagaaaagagggcagaggagtggtgagaatgatggcagggagcagagaaaagcactaaaattagagtcctgagagagaggcacaaggacatagaaacatggagatgtggggatgaattgcagagattccaaagagaactagagagaccgagaggcagagcaagacagatgatagatggatagatatagatagatgataaataggtagatgatagataataggttaaagatacatagatgatgattgattgattcattaatagataatacatagagatgatgatgatgaagacagataatacgtacagatagagaggcagacagaaatcatagagagagagatgatacatacatataaataacagatgattgatggatagatagacaagtgatagatacatagatgatatatagatatagatgacaggtagagaatttgtagataggcaccgaatagataaatagatagatcgacagataatagatagaaatatgcagaaagttatgaacaggacacaacgtgagaaacttagaatttaaaaaagtaacatcaagtcaaccaatccaaggagagtcagagagaataaaagaatccaaaaagggaaaacatatctagaggtggggaagcgaggtcagagacctagagagacagagaaggtggaagaaggaaatagacatgaagagagatggggtggagggtgagagagagagagagagagagcattaggtcatagagcaggggagtgagttctcagctcaggtgaagggagctgtgacaaggaagatcctccgtaaggaaaatgcctcttctcctccaggtctatatgagaaaccttctctctcagcccagccgggccccacggttctggcaggagagagcgtgaccttgtcctgcagctcccggagctcctatgacatgtaccatctatccagggagggggaggcccatgaacgtaggttctctgcagggcccaaggtcaacggaacattccaggccgactttcctctgggccctgccacccacggaggaacctacagatgcttcggctctttccgtgactctccatacgagtggtcaaactcgagtgacccactgcttgtttctgtcacaggtgaggaaaccccatatctgtctcatgtcctatgatcctagagccttagctgaggagcttcctgctgatgatggagagaagcatggacagatgcagagagaagacgaagcttgggtgtgagggagggatcagggcacaggatggcagacagggcacctccaaaccctcctacacggcctgcatgaaggcccgcggccagggctccaggcacacaggcagatggagaaaacggtcaggagagacccagaggagagagactgggctcagtttgggaagatcagaggttccctcagcccctcaacattacccatttcccagaagcccatcctggcctctcacccacacagggatgtcatcaccagcaacccctacaccctttacttttgtttgaagaaatatttattgaggataaatatacctatatagcttaccacctttaacattttttttttttttgaggcagagtctagctctgtcccctatgctggagtgcagtggcacaatctcagctcactgcaacttccgcctcctgggttcaagtgattctcctgcttcagccacctgagtagctggtgctacaggcgcgcaccaccacgccaggctactttttgtatttttagtagagagggggtttcaccatgttggtcgagctggtctccaactcctgaccacgtgatccacccgcatctgcctcccaaagtgctgggattacaggcatgagccaccacgcccagccacatttaccatttttaagtgtaaagtctagtggtcataaatacatttatatatatatatatatatatatatatacacacacacacacatatataaacatatatatatatatatatatatatatatatatatttttttttttttttttttttttaccctccacccttttattcctggcctctggaagccaccattctactctctaccttcatgagatccaccttttagctctgtatatgggtgagaaatgggaatctttgtaatgacttccagttccatccatgtggctgcaaatatcaggatgttattctttctatggatgagtagtctccactgtgcgtatgtactacattctctctatccattcatccactgatgggcaggtaggttgactccacatcttggctactgtgaacagtgctgcaccaatcatacgagtgcagatatcacttcgatatattgatttactttcctttggatataaacccagtagtgaaattgctggatactatgaaagttctctttttagttattcgtttgttgttttgtttttgtttttgagacagtttccctctgtgcccaggctggagtacaagtgaagtcatcttggctcattgcaacctccgcctcctgggttcaaatgattttcctgcctcagcctccctagtagctgggattacaggtgcacgccaccatgcctggctactttttgttttttttagtatagatggggtttccccatgttggctgggctgctctcaaactcatgacctcaactgaggtgcccgcctcggtctcccaaagtgccgggattacaggcatgatccacctcacccaacctctttttagttctttaaaggacttccacacttttctccgtaaaggctgtactaatttacactcctaccaacagggtattagggttctcctttctctaccactttggcaggatttcctttgcctgtcttgcagctaaaagccattttattttatttcattttattttgagatggagtttcgctcttgtcacccaggctggagtgcagtggtgcgatctcggctcaccacaacctccacctcccaggttcaagcgattctcctgcctcagcctcccgagtagctggaattacaggcacacgccaccacgcccaactaaattttgtatttttagtagagacagtgtttcttcatgtgggtcagactggtctcaaactcccgaccttatgaggttcacccacctcaggctctcaaaggtctaggatgacagacgtgagccaccacgcccggcctaaaatccattttaatggggtgagatgaaaactcactttgattttaatttgtgtttctctgatgatgagtgaaactgagcactttttagtatgtggggaaatttcatgtgttttgctcctttttcaattaaatcgtttgttttattgagttgtttgagcttcttatatttctagttattaatcccatctcagatgcatagtttgcacatatttgctcccaatctgtgggttgtctcttcactttgttggtttatttttagcggtgcagaagttgcttagtttgaggtaatcccaatggtctatttttgcttcgattacttgtgttttgaaggtttaaaacaaaatgtcttccttcagacaaatgtcctggagcatttccccaatattttcttctacgtgtttcataggttcaggccttagactcacatctttaatccattttcatttgagttttgtgtatagtgacaggtagaggtgcagtttcattcctctgcatgtagatgtccaggtttccctgcactgtttattgaaaagactgtcctttcctgattgtgagttcttggcacctttgtcaaagtccattggatgggctgggcatggtggctgacacctgcaatttcagcactttgggagcccaaggcgggtggatcacctgaggccaggagttcaagattagtctggccgacgtgatgaaacattgtctccactaaaaatataaaaattagctgagcatggtggtcagcacctgtaataccactactcaggagtttgaggccagagaattgattgaacccaggaggctgtggtggcagtgaaccgagattgcacctctgcactccagcctgggtgacagagcgagactccatctcaaaagaaaaaagaaaaaaacattggaggtaaatgcatggattatatctgtgttcttcattctgctccattgttctacgtgcctttctttatgccaatgtgatgctgttttgcttactacagctctgtaacatattttgagatcaggtagtgtgatgctcctgttttctctttataccttgaagtctcaagacagtgggcgtcacatacaaaaattacggaaaaaaggatcccaggactcccagggcccaatattagataacagagtgttggccatgaaccaacctcaaagatttccattgagtagaggacagacaccctcatttcctcacctctctcctgtctcgtgttctaggaaacccttcaaatagttggccttcacccactgaaccaagctccgaaaccggtgagtacagaaccctcttatatccgcttttggaaacctggggaggtagaaaccttcgatgcaggcattgactcagcatctcgcagctctgacattgtacgcctgtcttctaccatctccgaactccagatactccaacagcgaaagggatctgggcccaacctagggctcagtgaaatctcttaatctctcattttatggagctgagacctcctacaagctagaagaatgattgccaatctgacatccttctcaggaaaaatgcaatgtttgttctgcctgcattcctaactggaggataaattcctgggggcttgagagagggaagggaagggaacatctgatgagggcgaggtgttttagagaagttccacttgccaaggaatgaattactgttggtcatgaagcaaccctggctgactcagcagagcaacagccttgccgtaacagagaacggagctcatgcacgcacacttcgactcactgactcattcagccacggccccatgctcaggctgtgcagtgcggaaccttttcctattgttgccataacaaatttccacaagattcgtgggtgaaaacaaaacggttttttaattatcttacagtgctgtagctcaaagtaggaagtgcatcttactgggctaaaatcaaggtgacagcaaggctgccttccctctgaggattccaggcaagaatctgcttctcacttatcccagcttctaaaggctcccagttccttggctcctgttccccttcctccttcctcaaagcccacaaagactggtcacatctcacatggcatcactcagtgccttcttccttaccacacctctttctctgaatgctgctctcccttcttccttatcttttgaaaacttggggattctattgggttcaccaagatgaaaatccctcataatctcctggaaatcatccaggatacccttgttttaagttcagctgattagcaaccgcaattccatctacaatcttcattcctcctttccatgtaaaataacatattcacaagctatggaggctaggacagggacattttggggtgggacagcattctcctgccttccacaaacggtgaacaagatgcatttggcttctgcccttgggacactgatattgcagatggttaaatgggagggcagaaaatgaatgcacaagtggatctataaatgaatgatccattgggaagcatctgtgcatgaaatctattttttgtttgttcttttgtttattgagacagagtcgccctctgtcttccaggctacagtgcagtgtcacgatcttggctcactgcaacctgcgtctcctggattcaagtgattctcctgcctccgcctctcgagtagctgggattacaggcaactgccaccgtgcccggctaattctttttgtatattttttgtagagaggatgtttcaccacgttggccaagcttgtctgaaactcccaacctcaagtgatccgaccgtctcagcatgccaaagtaatgggactacaggcgtgagccactgtgcccagccagaattcaaaatcaataatagataatgctgagtgtatgatttcaggtgacaaagaaggtctcactattcagatatttgtgacattaatgaaaaacacggattgaacccctgaaagattggcggaaggattttgcacacacagctgtcagccgtgaaggcacaaaggtgaaaacaatctgatgtggaaggaagaggctctgcctcaaatgctgggaatgatgtggggagaatgacaagacgactgtagagagacggagagcacactgggtacacaggaaactaaggagcaacaaggagtgtgtgtttgacactcacagccattggattcacctcggggtaaccaggaatccctacatgattaatatgactgacatgaaaataagggaggctcagttgcataactggaatctaggagaccgtggaaaaggcaattgccaccccactggtgaaatgtggtgctgatttagacactaaatgaatgaagtagatggatataagatatgtttgtgaggtagaatcattgactggaaacgcttactgggtttgattttcctacttgtttaatcctcgcttaattaatttctttctgagatttattcatcctacacataaatcaatacctggcaaaggagtgacagatatatgagtggtggtggaaatgaagagacttattatagcataatatacaagtctgtgaacagtggctcacgcctgtaacctagcactgcaggaggccaaggtgggtggattccatgaagtcaggagttccagaccagcctggccaacgtggtgaaaccctatctctactaaaaatacaaaaattagccgagcacgatggtgcatccctgtaatcccagctcctattctggaggatgaagcaggagaatgacttcaacccagtaggtggaggttgcagtgagtggagattgcatcactgcactccagcctgggggacacaaggagactctatctcaaaaaataaaaataagaaatacataaatataataaaacacacacgaatgacaaaggcacctgaattccaatcatcgtttttctatttctctataattacttctttgatcctttatcttatccattaggcaatgagcttaaaacctcttccctatttggctttctgtgagaatgagatcacatagaaaatgtgaaagccctcagaatcctccagcacagatcgtggaatagagaaagtgctctgttcatcgcaacaaaaaacttgcccactcacccaaatcccccacctcacccctacttccaatcacctgtggagattcagataggctatggggaggtaaacattgatactccttggagtgagtccagatcttggaatcagagatcagtgccagcactagctcctgctcccctttcctactaattcacaggaggacaggtggtattgaagcaatagatggccgagggggtggtccttcccccagcctctcgggtagaacagcagcctaacatgtgtctcccgagatcacaaagagtagcacgtttcacacgggcttcaacactatttcctggccatttgacataagagaattctacttagctttttttatcttgatttcacttttgtttccttttcttggagaatgcaagttgtttgattcaagaatgctgtggatgtagaaatcctaaagcacattcgctgtgtatcaatcccagtgcagtcttcccagagaagactctaaatacctcctggactgcacctgggcttatgccaattcctatcactcaccgtcactccagggagacagaacacacagagaatacattacacaggcaggttcattactaacagataagcagcgagtgacaacagaaacctacatttcaatgtgagccagtccctcaaggctcagaaaagctactcgggacatatggagtcaccccatttgcagtgtagctgggggaagccagagagcagcccagcctgggttttgtactgtggagccacaggaagcactcagctaaagcactgcatgacgtcctcctccaggaagaacaggaagacagcccaggctgttctgagacgttcctcctgatctcaggacgttgctgtcttagtccatttttgttgctctaaaggaacacttgagcctgggtaacttctagagaaaagagattggtttgcctcacagttctgcaggctgtactggaagcgtggcaccagcatctatttctcgtgacggcctcaggctgctcccactctggcagaagggaaggagggtctgtctgtgcagagaccacagagatcacacggcaagagagggagcaagggggagggggagcgatggagcttccaagctcttttgaacaaccagctctccaggaactaatagaaggggaacttgctaaccccgtctccttgggacagcattggtctgttcatgatggatccacctccatgacccaaacacctctcaagaggcccaacctcccacagtgggggtgaaatttcaatgtgaggtttgaaggggtcaaacatctcaactaaagtagttgtatcctcaacacgttctatggttactatgagagctataactgagaaagcaggagaaagctgggtctccctccatctgggtgcttgtcctaaaggggtgttgtatgtggttacctgtcaatcaagaaatgtgagacaattcataaagaggaactgctatgattagcttcttattggtgtctcctcttcttccaggtaaccccagacacctgcatgttctgattgggacctcagtggtcatcatcctcttcatcctcctcctcttctttctccttcatcgctggtgctgcaacaaaaaaagtaagtctcacgaagcagaggccagagagctcagggccatgtggggaagcaggatgggagcactcaggtgtgtgttcctcacagacaggatggtccctggcccaaggcagcagccacagagggaggactttctagagagagcaccagactccctgtccctgccttcagctcacagaccattgcctgattctgaactgtatcctcatgtcccctgcagccactcacatccaggagaaggttccatgacaggcagaaagtgggagacagaatcaatgggatgggaactcagagctattcatgggatgggtccttgagctcagagagatagaatgtctgagtctgctgttggcaactgagggacctcaggctcctatggtctccccctgtatgttggtatctgcttatgaaatgagggcccagaagtgccctctgagctgttttgttgacttccgtcttctacagatgctgttgtaatggaccaagagcctgcagggaacagaacagtgaacagggaggtaggtgctcctcggcccagcctcgtggctagtgttattcccaaagagtcctggaaaatgtgagcaccctccctcactcagcatttccctctctccaggactctgatgaacaagaccctcaggaggtgacatatgcacagttgaatcactgcgttttcacacagagaaaaatcactcgcccttctcagaggcccaagacacccccaacagatatcatcgtgtacacggaacttccaaatgctgagccctgatccaaagttgtctcctgcccatgagcaccacagtcaggccttgaggggatcttctagggagacaacagccctgtctcaaaactgggttgccagctccaatgtaccagcagctggaatctgaaggcgtgagtctgcatcttagggcatcgctcttcctcacaccacaaatctgaacgtgcctctcccttgcttacaaatgtctaaggtccccactgcctgctggagagaaaacacactcctttgcttagcccacaattctccatttcacttgacccctgcccacctctccaacctaactggcttacttcctagtctacttgaggctgcaatcacactgaggaactcacaattccaaacatacaagaggctccctcttaacacggcacttagacacgtgctgttccaccttccctcatgctgttccacctcccctcagactagctttcagccttctgtcagcagtaaaacttatatattttttaaaataatttcaatgtagttttccctccttcaaataaacatgtctgccctca

SEQ ID NO: 625 - DNA, 1592 bp, Homo sapiens
  source 1..1592: mol_type = other DNA
ggggcgcggccgcctgtctgcacagacagcaccatgtcgctcatggtcgtcagcatggtgtgtgttgggttcttcttgctgcagggggcctggccacatgagggagtccacagaaaaccttccctcctggcccacccaggtcccctggtgaaatcagaagagacagtcatcctgcaatgttggtcagatgtcaggtttcagcacttccttctgcacagagaagggaagtttaaggacactttgcacctcattggagagcaccatgatggggtctccaaggccaacttctccatcggtcccatgatgcaagaccttgcagggacctacagatgctacggttctgttactcactccccctatcagttgtcagctcccagtgaccctctggacatcgtcatcacaggtctatatgagaaaccttctctctcagcccagccgggccccacggttctggcaggagagagcgtgaccttgtcctgcagctcccggagctcctatgacatgtaccatctatccagggagggggaggcccatgaacgtaggttctctgcagggcccaaggtcaacggaacattccaggccgactttcctctgggccctgccacccacggaggaacctacagatgcttcggctctttccgtgactctccatacgagtggtcaaactcgagtgacccactgcttgtttctgtcacaggaaacccttcaaatagttggccttcacccactgaaccaagctccgaaaccggtaaccccagacacctgcatgttctgattgggacctcagtggtcatcatcctcttcatcctcctcctcttctttctccttcatcgctggtgctgcaacaaaaaaaatgctgttgtaatggaccaagagcctgcagggaacagaacagtgaacagggaggactctgatgaacaagaccctcaggaggtgacatatgcacagttgaatcactgcgttttcacacagagaaaaatcactcgcccttctcagaggcccaagacacccccaacagatatcatcgtgtacacggaacttccaaatgctgagccctgatccaaagttgtctcctgcccatgagcaccacagtcaggccttgaggggatcttctagggagacaacagccctgtctcaaaactgggttgccagctccaatgtaccagcagctggaatctgaaggcgtgagtctgcatcttagggcatcgctcttcctcacaccacaaatctgaacgtgcctctcccttgcttacaaatgtctaaggtccccactgcctgctggagagaaaacacactcctttgcttagcccacaattctccatttcacttgacccctgcccacctctccaacctaactggcttacttcctagtctacttgaggctgcaatcacactgaggaactcacaattccaaacatacaagaggctccctcttaacacggcacttagacacgtgctgttccaccttccctcatgctgttccacctcccctcagactagctttcagccttctgtcagcagtaaaacttatatattttttaaaataatttcaatgtagttttccctccttcaaataaacatgtctgccctca

SEQ ID NO: 626 - protein, 341 aa, Homo sapiens
MSLMVVSMVCVGFFLLQGAWPHEGVHRKPSLLAHPGPLVKSEETVILQCWSDVRFQHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHERRFSAGPKVNGTFQADFPLGPATHGGTYRCFGSFRDSPYEWSNSSDPLLVSVTGNPSNSWPSPTEPSSETGNPRHLHVLIGTSVVIILFILLLFFLLHRWCCNKKNAVVMDQEPAGNRTVNREDSDEQDPQEVTYAQLNHCVFTQRKITRPSQRPKTPPTDIIVYTELPNAEP

(19) United States
(12) Patent Application Publication
(10) Pub. No.: US 2026/0357205 A1
(43) Pub. Date: Nov. 12, 2026

(54) INHIBITION OF KIR2DL3 FOR THE ENHANCEMENT OF ADOPTIVE IMMUNOTHERAPIES

(71) Applicant: H. LEE MOFFITT CANCER CENTER AND RESEARCH INSTITUTE INC., Tampa, FL (US)

(72) Inventors: Daniel Abate Daga, Tampa, FL (US); Miguel Gomez Fontela, Thousand Oaks, CA (US)

(21) Appl. No.: 19/099,304

(22) PCT Filed: Jul. 27, 2023
(86) PCT No.: PCT/US2023/071087

§ 371 Date: Jan. 28, 2025

Related U.S. Application Data
(60) Provisional application No. 63/369,701, filed on Jul. 28, 2022.

(51) Int. Cl. C12N 5/0783; A61K 35/17; A61K 40/11; A61K 40/31; A61P 37/04; C07K 16/28; C12N 15/11; C12N 15/113

(52) U.S. Cl. CPC C12N 5/0636; A61K 35/17; A61K 40/11; A61K 40/31; A61P 37/04; C07K 16/2818; C07K 16/2827; C12N 15/111; C12N 15/1138; C12N 2510/00

(57) ABSTRACT

Disclosed herein is a method for enhancing adoptively transferred autologous or allogeneic immune effector cells (T cells) in patients who are HLA-C1+. In some embodiments, the method involves ablating KIR2DL2 and/or KIR2DL3 expression in the T cells prior to adoptive transfer. In some embodiments, the T cells are further engineered to express a CAR. Therefore, disclosed herein are enhanced CAR-T cells that are engineered to have ablated KIR2DL2 and/or KIR2DL3 expression or activity.

### CROSS-REFERENCE TO RELATED APPLICATIONS

This application claims benefit of U.S. Provisional Application No. 63/369,701, filed Jul. 28, 2022, which is hereby incorporated herein by reference in its entirety.

### SEQUENCE LISTING

This application contains a sequence listing filed in ST.26 format entitled “320803-2910 Sequence Listing” created on Jun. 19, 2023, having 715,239 bytes. The content of the sequence listing is incorporated herein in its entirety.

### BACKGROUND OF THE INVENTION

Surgery, radiation therapy, and chemotherapy have been the standard accepted approaches for treatment of cancers including leukemia, solid tumors, and metastases. Immunotherapy (sometimes called biological therapy, biotherapy, or biological response modifier therapy), which uses the body's immune system, either directly or indirectly, to shrink or eradicate cancer has been studied for many years as an adjunct to conventional cancer therapy. It is believed that the human immune system is an untapped resource for cancer therapy and that effective treatment can be developed once the components of the immune system are properly harnessed.

### SUMMARY OF THE INVENTION

Disclosed herein is a method for enhancing adoptively transferred autologous or allogeneic immune effector cells (T cells) in patients who are HLA-C1+. Therefore, in some embodiments, a sample from the subject is assayed to determine their HLA profile and treated with the disclosed enhanced T cells if determined to be HLA-C1+. In some embodiments, the method involves ablating KIR2DL3 expression in the T cells prior to adoptive transfer. In some embodiments, the T cells are further engineered to express a CAR. Therefore, disclosed herein are enhanced CAR-T cells that are engineered to have ablated KIR2DL2 or KIR2DL3 expression or activity alone or KIR2DL3 and KIR2DL2 expression or activity.

Also disclosed are isolated nucleic acid sequences encoding the disclosed polypeptides, vectors comprising these isolated nucleic acids, and cells containing these vectors. The lymphocytes disclosed herein can be an immune effector cell selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell. In some embodiments, the lymphocytes are TILs.

Also disclosed is a method for treating a subject with adoptive cell therapy, in a subject, e.g. providing an anti-cancer immunity, that involves administering to the subject an effective amount of an enhanced T cell disclosed herein. Therefore, disclosed are methods of treating cancer in a subject that involves collecting lymphocytes from the subject, treating the lymphocytes ex vivo to inhibit KIR2DL2 and/or KIR2DL3 expression or activity, and transferring the modified lymphocytes back to the subject.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIGS. 1A and 1B show design, validation, and testing of the strategy for KIR2DL2/3 abrogation in primary human T cells. A strategy was designed for the abrogation of KIR2DL2/3 expression during CAR-T cell manufacturing. It was validated using an in vivo model of NSG mice bearing acute lymphoblastic leukemia grafts. FIG. 1A is a schematic representation of the gene editing validation protocol. First, the KIR2DL2 and KIR2DL3 loci were analyzed for the presence of guide RNAs (gRNAs) with the highest cutting score (1). Due to the low expression of KIR2DL2 within PBMCs, the KIR2DL2/3+ population was enriched by fluorescent activated cell sorting (2). KIR2DL2+ T cells were then nucleofected (Neon Transfection System, Thermo Fisher Scientific) with either a non-targeting guide RNA (NTC gRNA) or a gRNA targeting KIR2DL2 exon 1 (3). KIR2DL2/3 expression was analyzed 48 hours after nucleofection by flow cytometry (4). FIG. 1B is a flow cytometry histogram of a representative experiment (left panel) and KIR2DL2/3 mean fluorescence intensity (MFI, right panel) comparisons between pre-sorted, unedited (NTC) and KIR2DL2/3-ablated T cells. Data are shown as a mean±SD of three independent experiments.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in °C, and pressure is at or near atmospheric. Standard temperature and pressure are defined as 20 °C and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### DEFINITIONS

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### KIR2DL3 Ablation or Inhibition

In some embodiments, immune effector cells are modified ex vivo to inhibit or ablate KIR2DL2 and/or KIR2DL3 expression, and then adoptively transferred back to the subject.

As used herein the terms “inhibit” and “ablate” connote a partial or complete reduction in the expression and/or function of the KIR2DL2 and/or KIR2DL3 polypeptide encoded by the endogenous gene. Thus, the expression or function of the KIR2DL3 gene product can be completely or partially disrupted or reduced (e.g., by 50%, 75%, 80%, 90%, 95% or more, e.g., 100%) in a selected group of cells (e.g., a tissue or organ) or in the entire animal.

KIR2DL3 is a co-inhibitory receptor that may act as an immune checkpoint mechanism in the modulation of T cell activity. Its inhibitory effect is well characterized in natural killer (NK) cells, but little is known about its mechanism of action in T cells.

Multiple technologies exist to induce disruption of a gene, including CRISPR/Cas-based system, TALEN, MegaTAL, or other sequence-specific nucleases. In addition to genetic ablation using nucleases, other methods can be used to achieve reversible inhibition of KIR2DL2 and/or KIR2DL3 expression or activity. For instance, CRISPR/Cas systems can be engineered to induce reversible inhibition of gene transcription. Alternatively, small molecules or monoclonal antibodies could be dosed in vivo to block the interaction between KIR2DL2 and/or KIR2DL3 and its ligand. Small molecule inhibitors that target the phosphatase SHP1 could prevent KIR2DL2 and/or KIR2DL3 inhibition. Each of these and other approaches have different advantages and disadvantages. For example, genetic ablation has the advantage of inducing a permanent disruption in this pathway only on adoptively transferred T cells, which may allow for long-term prevention of KIR2DL2- and/or KIR2DL3-induced inhibition without affecting other KIR2DL2- and/or KIR2DL3-expressing cells in the immune system. On the other hand, transient inhibition with small molecule inhibitors or antibodies may allow for real-time control of the magnitude and duration of the checkpoint blockade.

In some embodiments, the ex vivo KIR2DL2 and/or KIR2DL3 ablation involves contacting the immune effector cells with a targeted nuclease, a guide RNA (gRNA), an siRNA, an antisense RNA, microRNA (miRNA), or short hairpin RNA (shRNA).

In some embodiments, the targeted nuclease may introduce a double-stranded break in a target region in the KIR2DL2 and/or KIR2DL3 gene of the immune effector cells. The targeted nuclease may be an RNA-guided nuclease. In some embodiments, the RNA-guided nuclease is a Cpf1 (Cas12) nuclease or a Cas9 nuclease and the method further comprises introducing into the immune effector cell a gRNA that specifically hybridizes to the target region in the KIR2DL2 and/or KIR2DL3 gene. In some embodiments, the Cas12 nuclease or the Cas9 nuclease and the gRNA are introduced into the cell as a ribonucleoprotein (RNP) complex. Therefore, in some embodiments, the ex vivo KIR2DL2 and/or KIR2DL3 ablation involves performing clustered regularly interspaced short palindromic repeats (CRISPR)/Cas genome editing.

In some embodiments, KIR2DL2 has the amino acid sequence: mslmvvsmacvgffllqgawpHEGVHRKPSLLAHPGRLVKSEETVILQCWSDVRFEHFLLHRE GKFKDTLHLIGEHHDGVSKANFSIGPMMQDLAGTYRCYGSVTHSPYQLSAPSDPLDIVI TGLYEKPSLSAQPGPTVLAGESVTLSCSSRSSYDMYHLSREGEAHECRFSAGPKVNG TFQADFPLGPATHGGTYRCFGSFRDSPYEWSNSSDPLLVSVTGNPSNSWPSPTEPSS KTGNPRHLHiligtsvviilfillfflIHRWCSNKKNAAVMDQESAGNRTANSEDSDEQDPQEV TYTQLNHCVFTQRKITRPSQRPKTPPTDIIVYTELPNAESRSKVVSCP (SEQ ID NO:1, NP_055034), comprising a signal peptide (lower case and italicized), EXTRACELLULAR DOMAIN (capitalized), transmembrane domain (lower case and bold, and INTRACELLULAR DOMAIN (capitalized and bold).

In some embodiments, KIR2DL2 gene has the nucleic acid sequence:

```
(SEQ ID NO: 2)
        1 cgcggccgcc tgtctgcaca gacagcacca tgtcgctcat ggtcgtcagc atggcgtgtg
      61 ttggtgagtc ctggaaggga atcgagggag ggagtgcggg gatggagatc ggggcccaga
    121 gttggagata taggcctgga agtggagtta tgggcctaga gatggagtga tgggcctaga
    181 agtggagatc tgggcctgga gtggagatat gggcctggag gttgagatat gggcctgcag
    241 tagagatatg ggcttgtagt ggagacatgg gcctggagat ggagatatgg gcctggagat
    301 ggagatatgg gcctgcagta gagatagggg cctggagtgg agatatgggc ctggagtgga
    361 gatatgggcc tgaagtggag atatgggcct ggaggtggag atatgggcct ggaggtggag
    421 atatgggcct ggagtggaga tatgggtctg gaggtggaga tacgggcctg cagtagagat
    481 atgggcctgg agtggagata tgggccagga gtggagttat gggcctagag gtggatatct
    541 gggcctggag tggagatatg ggcctaggaa ggagatatgg gcctgggtgt ggagatatgg
    601 gactggagag gtgatatggg cctggagtgg agatatgggc ttagggtgga gttctgggcc
    661 tggggcggag atatgggact ggattggaga taggggccta gggtggagat ctgagcctgg
    721 attggcgata tgggcctagg gtggaaatat cagcctggag tggagatatg ggcttggggt
    781 ggggatatgg gcctggaaac tgggtctctg cacagccgac agccctgttc ttgggtgcag
    841 gtaggcactg agggtgagtt taacttcagc ccaggaaggg cctggctgcc aagactcaca
    901 gcccagtggg ggcagcaagg gagggctggt tcgcctgcag atggatcgtc catcatgatc
    961 tttctttcca gggttcttct tgctgcaggg ggcctggcca catgagggtg agtccttctc
  1021 caaaccttcg ggtgtcatct ccccacataa gaggattttc ctgaaacagg agggaagtcc
  1081 tgtcggggag tctctcataa actaggaaga gaggaccctg gggtgctcag cccacatttc
  1141 tgacctcgcc tccctggcct ctcaacccct tggcagagtc aagttctgtg gggaccaggg
  1201 ttagactggg gtgctcaaag ctggggtgtg tggttgggaa gtggtaggaa cagcagatcc
  1261 tctgaggaca aaggtgttac tcacacactt cagcgtttcc atgatggtag gggctgcagt
  1321 gtggctgctg tcattctacc agaagaggtg ggaaaccaca gccatggccc tgacattcca
  1381 aatcctctga tgggggctca gttgtttatt ttcgttcagg catccgctga tatccattca
  1441 caaaggacat gccctccacc tcatgtctac cctgtgttgt tttatgtgag taatcttaca
  1501 gtatcaaaat ctagtaggag tctctttact cagcacttgc tcaaagttct cagctgaggc
  1561 ttttgttgta gggagacacc atgtctttgc gggatgggtc cttccttcag ccctgggcac
  1621 caaggtgtga tagtagccat agaaacgtgg aaagcgagga gaatcttctg agcacaggga
  1681 gggaggggca gttccacatc ctcctctcta aggcggcgcc tccttctccc caaggtggtc
  1741 aggacaagcc cttgctgtct gcctggccca gccttgtggt gcctctagga catgtcattc
  1801 ttcggtgtca ctcttatctt gggtttaaca acttcagtct gtacaaggaa ggtggggtgc
  1861 ctgtccctga gctctacaac agaatattct ggaacagcct tttcatgggc cctgtgaccc
  1921 ccgcacaaca gggacataca gatgtcgggg ttcacacaca cactccccca gtgggtggtc
  1981 agcacccagc aaccccctgg tgatcgtggt cataggtcag agggctcctg tcttggattc
  2041 tccttgtccc acctcctgaa tcccagagct tctggtgggc atgtccttga gggtcccatc
  2101 acgcaggccc tgactgtatt tgtggtaaag ggggattgaa tacagggaaa tgggtgctgt
  2161 ggtgggaaga ataattgtcc ccagtgatga ctacattcta atccctggag tctgtgacta
  2221 tgtatgttat aggggaaggg actgaagggg aagatggagc tcatggggag acagcctgga
  2281 ctgtcccact gggctcagtg taatcacaag ggtgcacatg aaaggaggag gaagagggga
  2341 gtggggatta gagcagtcca gtggaagtct tcaccagctt tgaaggtgga ggaaggccaa
  2401 gagccatgaa tgcaggtggc ctatagaggc tggaaaagtc aaggaactga ttctccagag
  2461 tctccagagg gaacaaagcc ctgcagatgc cttgatttta gcccaggaaa aatagggtcc
  2521 aatttctgtc tccagtactg gaaggtgtca gtgtggtctc tcctgcttcc atgcttctga
  2581 taattttgta cagcagcaac aggaaaccaa cactggaacc caggtcaagg acaagttaag
  2641 aaacaaccca aggaaagcca ggcatggtgg caggcgcatg taatcctagc gactcaggag
  2701 gctgagggca ggagaatcac ttgaacccag gaaacagagg ttgcagtgag cctagaccac
  2761 accacttcac tccagcctgg gtgaaggagt gagactctgt ctccaaaatt aattaattaa
  2821 ttaaagaaac caaagaagga gaaggttggc taccctgaga tcagcaaggg tgggatgatg
  2881 atgccaccac caggctccat ccacataggg aggggttgat actcctccaa ccagcaccag
  2941 gagccagcct atggaagctg gcaccatgga gaaggcacag gcatggcaag agtggctccc
  3001 agtccccacc aggaacaggg tgtgtggaca ctggtgcctg ccttattcat cagttcatat
  3061 cttctgccaa ggattgcaat tcatccaaaa gagattgaac caggctgata agagcctgga
  3121 tgtgcagcct atcctggttc ctctttcacc cccacataaa cagcaggaaa gacattagtg
  3181 tgaaatagat acaacacccc aagagatgag gctaagccca gtgggaaggg aatcagaggc
  3241 tactagagac agagggacag agaagaggga gggagacaga tggaaggacc tgcaccagga
  3301 gttaagggca cagaaaagaa catgaagaca cagagaggaa ggagagagac agacaccagc
  3361 aaggggaagc ctcactcatt ctaggtgcca tggatgggat gataaagaga gacaccttct
  3421 aaactcacaa cctctcttcc taggagtcca cagaaaacct tccctcctgg cccacccagg
  3481 tcgcctggtg aaatcagaag agacagtcat cctgcaatgt tggtcagatg tcaggtttga
  3541 gcacttcctt ctgcacagag aagggaagtt taaggacact ttgcacctca ttggagagca
  3601 ccatgatggg gtctccaaag ccaacttctc catcggtccc atgatgcaag accttgcagg
  3661 gacctacaga tgctacggtt ctgttactca ctccccctat cagttgtcag ctcccagtga
  3721 ccctctggac atcgtcatca caggtgagag tgtccggaca ttctcattgt cattgggctg
  3781 cagagtgaat gatccacgac ttggaacccc caggtagttg taaggaagat gagcttggta
  3841 ttcttatgga gagagactga cttgctgagg tttgtaccaa cagagacaga gaaacaggag
  3901 acacaagtac agaccaggtg tcataacgga ggacagacac aggggccata cagggagtta
  3961 gaaaagacag aaagagttaa aagagacaga cagacagaca tgtcccagag agaggtgtcc
  4021 ctccatgctg actttgctca cagacctggc acaggttaga agtttcattt ctgttttacc
  4081 tccacaaagt gttctctacc aggagaaccc aaggacaccc atatttctga cctgagttgg
  4141 gccctgtggc ctcaggcctt gtggcaccta caggccatgt ttattctgac acctctgcct
  4201 tccatgtaat ggagagtaac cgtcccagga tatcatggcc ccagaacacc aacccctgta
  4261 tgctgtgtga acttgtggtc tccagactgg attctgaggc tcacattcca aataacccca
  4321 catatgaaag gatcactgag aggcacagag aaaaatcagg aacaccaaaa agcaaagaca
  4381 taaacacacg gagaatgagc cagaggaagg agattgagag actcacagac acataaagag
  4441 agagaaaaga gggcagagga gtggtgagaa tgatggcagg gagcagagaa aagcactaaa
  4501 attagagtcc tgagagagag gcacaaggac atagaaacat ggagatgtgg ggatgaattg
  4561 cagagattcc aaagagagct agagagaccg agaggcagag caatacagat gatagatgga
  4621 tagatataga tagatgataa ataggtagat gatagataat aggttaaaga tacatagatg
  4681 atgattgatt gattcattaa tagataatac atagagatga tgatgatgaa gacagataat
  4741 acgtacagat agagaggcag acagaaatca tagagagaga gatgatacat acatataaat
  4801 aacagatgat tgatggatag atagacaact gatagataca tagatgatat atagatatag
  4861 atgacaggta gagaatttgt agataggcac cgaatagata aatagataga tcgacagata
  4921 atagatagaa atatgcagaa agttatgaac aggacacaac gtgagaaact tagaatttaa
  4981 aaaagtaaca tcaagtcaac caacccaagg agagtcagag agaataaaac aatccaaaaa
  5041 cggaaaacat atctagaggt ggggaagcga ggtcagagac ctagagagac agagaaggtg
  5101 gaagaaggaa atagatatga agagagatgg ggtggagggt gagagagaga gagagagagc
  5161 attaggtcat agagcagggg agtgagttct cagctcaggt gaagggagct gtgacaagga
  5221 agatcctccc tgaggaaaat gcctcttctc cttccaggtc tatatgagaa accttctctc
  5281 tcagcccagc cgggccccac ggttctggca ggagagagcg tgaccttgtc ctgcagctcc
  5341 cggagctcct atgacatgta ccatctatcc agggaggggg aggcccatga atgtaggttc
  5401 tctgcagggc ccaaggtcaa cggaacattc caggccgact ttcctctggg ccctgccacc
  5461 cacggaggaa cctacagatg cttcggctct ttccgtgact ctccatacga gtggtcaaac
  5521 tcgagtgacc cactgcttgt ttctgtcata ggtgaggaaa ccccatatct gtctcatgtc
  5581 ctatgatcct agagccttag ctgaggagct tcctgctgat gatggagata agcatggaca
  5641 gatgcagaga gaagacgaag cttgggtgtg agggagggat cagggcacag gatggcagac
  5701 agggcacctc caaaccctcc tacacggcct gcatgaaggc ccgcggccag ggctccaggc
  5761 acacaggcag atggagaaag cggtcaggag agacccagag gagggagact gggctcagtt
  5821 tgggaagatc agaggttccc tcagcccctc aacattaccc atttcccaga agcccatcct
  5881 ggcctctcac ccacacaggg atgtcatcac cagcaacccc tacacccttt acttttgttt
  5941 gaagaaatat ttattgagga taaatatacc tatatagctt accaccttta acattttttt
  6001 tttttttgag gcagagtcta gctctgtccc ctatgctgca gtgcagtggc acaatctcag
  6061 ctcactgcaa cttccgcctc ctgggttcaa gtgattctcc tgcctcagcc acctgagtag
  6121 ctggtgctac aggcgcgcac caccacgcca ggctactttt tgtattttta gtagagaggt
  6181 ggtttcacca tgttggtcga gctggtctcc aactcctgac cacgtgatcc acccgcatct
  6241 gcctcccaaa gtgctgggat tacaggcatg agccaccact cccagccaca tttaccattt
  6301 ttaagtgtaa agtctagtgg tcataaatac atttataaat atatatatat atatatgtat
  6361 gtatatatat atatatatat atatatatat atatattttt tttttttttt ttaccctcca
  6421 cccttttctt cctggcctct ggaagccacc attctactct ctaccttcat gagatccacc
  6481 ttttagctct gtatatgggt gagaaatggg aatctttgta atgacttgca gttccatcca
  6541 tgtggctgca aatatcagga tgttattctt tctatggatg agtagtctcc actgtgcgta
  6601 tgtactacat tctctctatc cattcatcca ctgatgggca ggtaggttga ctccacatct
  6661 tggctactgt gaacagtgct gcaccaatca tacgagtgca gatatcactt cgatatattg
  6721 atttactttc ctttggatat aaacccagta gtgaaattgc tggatactat gaaagttctc
  6781 tttttagtta ttcgtttgtt gttttgtttt tgtttttgag acagtttccc tctgtgccca
  6841 ggctggagta caagtgatgt catcttggct cattgcaacc tctgcctcct gggttcaaat
  6901 gattttccta cctcagcctc cctagtagct gggattacag gtgcacgcca ccatgcctgg
  6961 ctactttttg gtttttttag tatagatggg gtttccccat gttggctggg ctgctctcaa
  7021 actcatgacc tcaactgagg tgtccgcctc ggtctcccaa agtgccggga ttacaggcat
  7081 gatccacctc acccaacctc tttttagttc tttaaaggac ttccacactt ttctccgtaa
  7141 aggctgtact aatttacact cctaccaaca gggtattagg gttctccttt ctctaccact
  7201 ttggcaggat ttcctttgcc tgtcttgcag ctaaaagcca ttttacttta tttcatttta
  7261 ttttgagatg gagtttcgct cttgtcaccc aggctggagt gcagtggtgc gatctcggct
  7321 caccacaacc tccacctccc aggttcaagc gattctcctg cctcagcctc ccgagtagct
  7381 ggaattacag gcacacgcca ccacgcccga ctaatttttg tatttttagt agagacagtg
  7441 tttctccatg tgggtcagac tggtctcaaa ctcccgacct tatgagattc acccacctca
  7501 ggctctcaaa gatctaggat gacagacgtg agccaccacg cccggcctaa aagccatttt
  7561 aatggggtga gatgaaaact cactttgatt ttaatttgcg tttctctgat gatgagtgat
  7621 actgagcagt ttttcgtatg tggggaaatt tcatgtcttt tgctcctgtt tcaattaaat
  7681 catttgtttt attgagttgt ttgagcttct tatatttcta gttattaatc ccatctcaga
  7741 tgcatagttt gcacatattt gctcccaatc tgtgggttgt ctcttcactt tgttggttta
  7801 tttttagcgg tgcagaagtt gcttagcttg aggtaatccc aatggtctat ttttgcttcg
  7861 attacttgtg ttttgaaggt ttaaaacaaa atgtcttcct tcagacaaat gtcctggagc
  7921 atttccccaa tattttcttc tacgtgtttc ataggttcag gccttagact cacatcttta
  7981 atccattttc atttgatttt tgtgtatggt gacaggtaga ggtgcagttt cattcctctg
  8041 catgtagatg tccaggtttc cctgcactgt ttattgaaaa gactgtcctt tcctgattgt
  8101 gagttcttgg cacctttgtc aaagtccatt ggatgggctg ggcatggtga ctgacacctg
  8161 caatttcagc actttgggag cccaaggcgg gtggatcacc tgaggccagg agttcaagat
  8221 tagtctggcc gacgtgatga aacatcgtct ccactaaaaa tatataaatt agctgagcat
  8281 ggtggtcagc acctataata ccactactca ggagtttgag gccagagaat tgattgaacc
  8341 caggaggctg tggtggcagt gaaccgagat tgcacctctg cactccagcc tgggtgacag
  8401 agcgagactc catctcaaaa gaaaaaagaa aaaaacattg gatgtaaatg catggattat
  8461 atttgtgttg ttcattctgc tccattgttc tatgtgcctt tcttcatgcc aacatcatgc
  8521 tgtcttgctt actacagctc tgtaacatat tttgagatca ggtagtgtga tgctcctgtt
  8581 ttctctttat accttgaagt ctcaagacaa tgggcgtcac atacaaaaat tatggaaaaa
  8641 aggatcccag gactcccagg gcccaatatt agataacaga gtgttggcca tgaaccaacc
  8701 tcaaagattt ccattgagta gaggacagac accctcattt cctcacctct ctcctgtctc
  8761 atgttctagg aaacccttca aatagttggc cttcacccac tgaaccaagc tctaaaaccg
  8821 gtgagtacag aaccctctta tatccgcttt tggaaacctg gggaggtaga aaccttcgat
  8881 gcaggcattg actcagcatc tcgcagctct gacattgtac gcctgtcttc taccatctcc
  8941 gaactccaga tactccaaca gcgaaaggga tctgggccca acctagggct cagtgaaatc
  9001 tcttaatctc tcattttatg gagctgagac ctcctacaag ctagaagaat gattgccaat
  9061 ctgacatcct tctcaggaaa aatgcaatgt ttgttctgcc tgcattccta actggaggat
  9121 aaattcctgg gggcttgaga gagggaaggg aagggaacat ctgatgaggg cgaggtgttt
  9181 tagagaagtt ccacttgcca aggaatgaat tactgttggt catgaagcaa ccctggctga
  9241 ctcagcagag caacagcctt gccgtaacag agaacggagc tcatgcacgc acacttcgac
  9301 tcactgactc attcagccac ggccccatgc tcaggctgtg cagtgcggaa ccttttccta
  9361 ttgttgccat aacaaatttc cacaagattc gtgggtgaaa acaaaacggt tttttaatta
  9421 tcttacagtg ctgtagctca aagtaggaag tgcatcttac tgggctaaaa tcaaggtgac
  9481 agcaaggctg ccttccctct gaggattcca ggcaagaatc tgcttctcac ttgtcccagc
  9541 ttctaaaggc tcccagttcc ttggctcctg gtccccttcc tccttcctca aaacccacaa
  9601 agactggtca catctcacat ggcatcactc agtgccttct tccttaccac acctctttct
  9661 ctgaatgctg ctctcccttc ttccttatct tttgaaaact tggggattct attgggttca
  9721 ccaagatgaa aatccctcat aatctcctgg aaatcatcca ggataccctt gttttaagtt
  9781 cagctgatta gcaaccgtaa ttccatctac aatcttcatt cctcctttcc atgtaaaata
  9841 acatattcac aaggtatgga ggctaggaca gggacatttt ggggtgggac agcattctcc
  9901 tgccttccac aaacagtgaa caagatgcat ttggcctctg cccttgggac actgatattg
  9961 cagatggtta aatgggaggg cagaaaatga atgcacaagt ggatctataa atgaatgatc
10021 cattgggaag catctgtgca tgaaatctat tttttgtttg ttcttttgtt tattgagaca
10081 gagttgccct ctgtcttcca ggctacagtg cagtgtcacg atcttggctc actgcaacct
10141 gcttctcctg gattcaagtg attctcctgc ctccgcctct cgagtagctg ggattacagg
10201 caactgccac cgtgcccggc taattctttt tgtatatttt ttgtagagag gatgtttcac
10261 cacgttggcc aagcttgtct gaaactccca acctcaagtg atccgaccgt ctcagcatgc
10321 caaagtaatg ggactacagg cgtgagccac tgtgcccagc cagaattcaa aatcaataat
10381 agataatgct gagtgtatga tttcaggtga caaagaaggt ctcactattc agatatttgt
10441 gacattaatg aaaaacacgg attgaacccc tgaaagattg gcggaaggat tttgcacaca
10501 cagctgtcag ccgtgaaggc acaaaggtga aaacaatctg atgtggaagg aagaggctct
10561 tcctcaaatg ctgggaatga tgtggggaga atgacaagat gactgtggag agacggagag
10621 cacactgggt acacaggaaa ctaaggagga acaaggagtg tgtgtttgac actcacagcc
10681 attggattca cctcggggta gccaggaatc cctacatgat taatatgact gacatgaaaa
10741 taagggaggc tcagttgcat aactggaatc taggagaccg tggaaaaggc aattgccgcc
10801 ccactggtga aatgtggtgc tgatttagac actaaatgaa tgaagtagat ggatataaga
10861 taggtttgtg aggtagaatc attgactgga aaggcttgct gggtttgatt ttcctacttg
10921 tttaatcctc gcttaattaa tttctttctg agatttattc atcctacaca taaatcaata
10981 cctggcaaag gagtgacaga tatatgaggg gtggtggaaa tgaagagacc tattatagca
11041 taatatacaa gtctgtgaac ggtggctcac gcctgtaacc cagcactgca ggaggccaag
11101 gcgggtggat cacatgaagt cagcagttcg agaccagcct ggccaacatg gtgaaaccct
11161 gtctctagga aaaacacaaa aattagccga gcatggtggt gcatccctgt aatcccagct
11221 cctactctgg aggatgaagc aggagaatga cttcaaccca ggaggtggag gttgcagtga
11281 gtggaggttg catcactgca ctccagcctg ggtggcacaa ggagactccg tctcaaaaaa
11341 taaaaataag aaatgcataa atataaatat aatataacac acgcaaatga caaagggacc
11401 tgaattccaa tcatgatttt tctatttctc tataattact tctttgatcc tttatcttat
11461 ccattaggca atgagcctaa aacctcttcc ctatttggct ttctgtgagc atgagatcat
11521 atagaaaatg tgaaagtccg ctgaatcctc cagcacagat cctggaatag agaaagtgct
11581 ctggtcatca caaaaaaaac ttgcccactc acccaaatcc cccacctcac ccctacttcc
11641 aatcacctgt ggagattcag gtagaccatg gggaggtaaa cattaacact ccttggagtg
11701 agtccagatc ttggaatcag agatcagcga cagcactagc tcctgctccc ctttcctact
11761 aattcacagg aggacaggtg gtattgaagc aatagatggc cgagggggtg gtccttcccc
11821 cagcctctcg ggtagaacag cagcctaata tgtgtctccc gagatcacaa agagcagcag
11881 gtttcacacg ggcttcaaca ctatttcctg gccgtttgac ataagagaat tctatttcgc
11941 tttttttatc ttgatttcac ttttgttttc tttccttgga gaatgcaagt tgtttgattc
12001 aagaatgctg tggatgtaga aaccctaaag cacattcgct gtgaatcaat cccagtccag
12061 tcttcccaga gaagactcta aacacctcct ggactgcacc tgggcctatg ccaattccta
12121 tcactcaccg tcactccagg gagacagaac acacagagaa tacgttacat aggcaggttc
12181 attactaaca gataagcagc gagtgacaac agaaacctat atttcaatgt gagccagtcc
12241 ctcaaggctc agaaaagctc ctcgggacat atggagtcac cccatttgca gtgtagctgc
12301 gggaagccag aaagcagccc agcctgggtt ttgtaccctg gagccacagg aagcactcag
12361 ctaaagcact gcatgacgtc ctccaggaag aacaggaaga cagcccaggg tgttctgaga
12421 cgttcctcct gatctcagga agttgctgtc ttaggccatt tttgttgctc taaaggaaca
12481 cttgagcctc ggtaacttct aaagaaaaga gattggtttg cctcaccgtt ctgcaggctg
12541 tactggaagc atggcaccag catctatttc tcgtgacggc ctcaggctgc tcccactctg
12601 gcagaaggga aggagggtct gtctgtgcag agaccacaga gatcacacgg caagagaggg
12661 agcaaggggg agggggagtg atggagcttc caagctcttt ttaacaacca gctctccggg
12721 aactaataga gggggaactt gctaaccccg tctccttggg acagcattga tgtgttcatg
12781 atggatccac ctccatgacc caaacacctc tcaagaggcc caacctccca cagtgggggt
12841 gaaatttcaa tgtgaggttt gaaggggtca aacatctcaa ctaaagtagt cgtatcctca
12901 gcacgttcta tggttactat gagagctata actgaaaaag caggagaaag ctgggtctcc
12961 tgccatctgg gtgcttgtcc taaagagatg ttttatgtgg ttacctgtca atcaagaaat
13021 gcgagacaat tcataaagag gaactgctaa gattagcttc ttattggtgt ctcatcttct
13081 tccaggtaac ccccgacacc tgcacattct gattgggacc tcagtggtca tcatcctctt
13141 catcctcctc ttctttctcc ttcatcgctg gtgctccaac aaaaaaagta agtctcacga
13201 agcagaggcc agagagctca gggccatgtg gggaagcagg atgggagcac tcaggtgtgt
13261 gttcctcaca aacaggatgg tccctggccc aaggcagcag ccacagaggc aggactttct
13321 agagagggca ccagactccc tgcccctgcc ttcaactcac agaccgttgc ctgattctga
13381 actgtatcct catgtcccct gcagccactc acatccagga gaaggttcca tgacaggcag
13441 aaagtgggag acagaatcaa tgggatggga actcagagct attcatggga tgggtccttg
13501 agctcagaga gatagaatgt ctgagtctgc tgttggcaac tgagggacct cagccaccta
13561 tggtctcccc ctgtatgttg gtatctgctt atgaaatgag gacccagaag tgccctccga
13621 gctgttttgt tgacttccgt ctcctacaga tgctgcggta atggaccaag agtctgcagg
13681 gaacagaaca gcgaatagcg aggtaggtac tcctcggccc gggctcgtgg ctactgttat
13741 tcccaaagag tcctggaaaa tgtgagcacc ctccctcact cagcatttcc ctctctccag
13801 gactctgatg aacaagaccc tcaggaggtg acatacacac agttgaatca ctgcgttttc
13861 acacagagaa aaatcactcg cccttctcag aggcccaaga cacccccaac agatatcatc
13921 gtgtacgcgg aacttccaaa tgctgagtcc agatccaaag ttgtctcctg cccatgagca
13981 ccacagtcag gccttgaggg cgtcttctag ggagacaaca gccctgtctc aaaaccgggt
14041 tgccagctcc catgtaccag cagctggaat ctgaaggcat gagtctgcat cttagggcat
14101 cgctcttcct cacaccacaa atctgaatgt gcctctcact tgcttacaaa tgtctaaggt
14161 ccccactgcc tgctggagaa aaaacacact cctttgctta gcccacagtt ctccatttca
14221 cttgacccct gcccacctct ccaacctaac tggcttactt cctagtctac ttgaggctgc
14281 aatcacactg aggaactcac aattccaaac atacaagagg ctccctctta acgcagcact
14341 tagacacgtg ttgttccacc ttccctcatg ctgttccacc tcccctcaga ctagctttca
14401 gtcttctgtc agcagtaaaa cttatatatt ttttaaaata acttcaatgt agttttccat
14461 ccttcaaata aacatgtctg cccccatg.
```

In some embodiments, the KIR2DL2 gene has the consensus sequence: gagcacccactgggcctcatgcaaggtagaaagagcctgcgtacgtcaccctcccatgatgtggtcaacatgtaaactgc atgggcagggcgccaaataacatcctgtgcgctgctgagctgagctggggCGCGGCCGCCTGTCTGCACA GACAGCACCATGTCGCTCATGGTCGTCAGCATGGCGTGTGTTGgtgagtcctggaaggga atcgagggagggagtgcggggatggagatcggggcccagagttggagatataggcctggaagtggagttatgggccta gagatggagtgatgggcctagaagtggagatctgggcctggagtggagatatgggcctggaggttgagatatgggcctgc agtagagatatgggcttgtagtggagacatgggcctggagatggagatatgggcctggagatggagatatgggcctgcag tagagataggggcctggagtggagatatgggcctggagtggagatatgggcctgaagtggagatatgggcctggaggtg gagatatgggcctggaggtggagatatgggcctggagtggagatatgggtctggaggtggagatacgggcctgcagtag agatatgggcctggagtggagatatgggccaggagtggagttatgggcctagaggtggatatctgggcctggagtggaga tatgggcctaggaaggagatatgggcctgggtgtggagatatgggactggagaggtgatatgggcctggagtggagatat gggcttagggtggagttctgggcctggggcggagatatgggactggattggagataggggcctagggtggagatctgagc ctggattggcgatatgggcctagggtggaaatatcagcctggagtggagatatgggcttggggggggatatgggcctgga aactgggtctctgcacagccgacagccctgttcttgggtgcaggtaggcactgagggtgagtttaacttcagcccaggaag ggcctggctgccaagactcacagcccagtgggggcagcaagggagggctggttcgcctgcagatggatcgtccatcatg atctttctttccagGGTTCTTCTTGCTGCAGGGGGCCTGGCCACATGAGGgtgagtccttctccaaa ccttcgggtgtcatctccccacataagaggattttcctgaaacaggagggaagtcctgtcggggagtctctcataaactagg aagagaggaccctggggtgctcagcccacatttctgacctcgcctccctggcctctcaaccccttggcagagtcaagttctgt ggggaccagggttagactggggtgctcaaagctggggtgtgtggttgggaagtggtaggaacagcagatcctctgagga caaaggtgttactcacacacttcagcgtttccatgatggtaggggctgcagtgtggctgctgtcattctaccagaagaggtgg gaaaccacagccatggccctgacattccaaatcctctgatgggggctcagttgtttattttcgttcaggcatccgctgatatcca ttcacaaaggacatgccctccacctcatgtctaccctgtgttgttttatgtgagtaatcttacagtatcaaaatctagtaggagtct ctttactcagcacttgctcaaagttctcagctgaggcttttgttgtagggagacaccatgtctttgcgggatgggtccttccttcag ccctgggcaccaaggtgtgatagtagccatagaaacgtggaaagcgaggagaatcttctgagcacagggagggaggg gcagttccacatcctcctctctaaggcggcgcctccttctccccaaggtggtcaggacaagcccttgctgtctgcctggccca gccttgtggtgcctctaggacatgtcattcttcggtgtcactcttatcttgggtttaacaacttcagtctgtacaaggaaggtggg gtgcctgtccctgagctctacaacagaatattctggaacagccttttcatgggccctgtgacccccgcacaacagggacata cagatgtcggggttcacacacacactcccccagtgggtggtcagcacccagcaaccccctggtgatcgtggtcataggtc agagggctcctgtcttggattctccttgtcccacctcctgaatcccagagcttctggtgggcatgtccttgagggtcccatcacg caggccctgactgtatttgtggtaaagggggattgaatacagggaaatgggtgctgtggtgggaagaataattgtccccagt gatgactacattctaatccctggagtctgtgactatgtatgttataggggaagggactgaaggggaagatggagctcatggg gagacagcctggactgtcccactgggctcagtgtaatcacaagggtgcacatgaaaggaggaggaagaggggagtgg ggattagagcagtccagtggaagtcttcaccagctttgaaggtggaggaaggccaagagccatgaatgcaggtggcctat agaggctggaaaagtcaaggaactgattctccagagtctccagagggaacaaagccctgcagatgccttgattttagccc aggaaaaatagggtccaatttctgtctccagtactggaaggtgtcagtgtggtctctcctgcttccatgcttctgataattttgtac agcagcaacaggaaaccaacactggaacccaggtcaaggacaagttaagaaacaacccaaggaaagccaggcatg gtggcaggtgcatgtaatcctagcgactcaggaggctgagggcaggagaatcacttgaacccaggaaacagaggttgc agtgagcctagaccacaccacttcactccagcctgggtgaaggagtgagactctgtctccaaaattaattaattaattaaag aaaccaaagaaggagaaggttggctaccctgagatcagcaaggggggatgatgatgccaccaccaggctccatccac atagggaggggttgatactcctccaaccagcaccaggagccagcctatggaagctggcaccatggagaaggcacagg catggcaagagtggctcccagtccccaccaggaacagggtgtgtggacactggtgcctgccttattcatcagttcatatcttct gccaaggattgcaattcatccaaaagagattgaaccaggctgataagagcctggatgtgcagcctatcctggttcctctttca cccccacataaacagcaggaaagacattagtgtgaaatagatacaacaccccaagagatgaggctaagcccagtggg aagggaatcagaggctactagagacagagggacagagaagagggagggagacagatggaaggacctgcaccagg agttaagggcacagaaaagaacatgaagacacagagaggaaggagagagacagacaccagcaaggggaagcct cactcattctaggtgccatggatgggatgataaagagagacaccttctaaactcacaacctctcttcctagGAGTCCAC AGAAAACCTTCCCTCCTGGCCCACCCAGGTCGCCTGGTGAAATCAGAAGAGACAG TCATCCTGCAATGTTGGTCAGATGTCAGGTTTGAGCACTTCCTTCTGCACAGAGAA GGGAAGTTTAAGGACACTTTGCACCTCATTGGAGAGCACCATGATGGGGTCTCCAA AGCCAACTTCTCCATCGGTCCCATGATGCAAGACCTTGCAGGGACCTACAGATGCT ACGGTTCTGTTACTCACTCCCCCTATCAGTTGTCAGCTCCCAGTGACCCTCTGGAC ATCGTCATCACAGgtgagagtgtccggacattctcattgtcattgggctgcagagtgaatgatccacgacttggaac ccccaggtagttgtaaggaagatgagcttggtattcttatggagagagactgacttgctgaggtttgtaccaacagagacag agaaacaggagacacaagtacagaccaggtgtcataacggaggacagacacaggggccatacagggagttagaaa agacagaaagagttaaaagagacagacagacagacatgtcccagagagaggtgtccctccatgctgactttgctcaca gacctggcacaggttagaagtttcatttctgttttacctccacaaagtgttctctaccaggagaacccaaggacacccatattt ctgacctgagttgggccctgtggcctcaggccttgtggcacctacaggccatgtttattctgacacctctgccttccatgtaatg gagagtaaccgtcccaggatatcatggccccagaacaccaacccctgtatgctgtgtgaacttgtggtctccagactggatt ctgaggctcacattccaaataaccccacatatgaaaggatcactgagaggcacagagaaaaatcaggaacaccaaaa agcaaagacataaacacacggagaatgagccagaggaaggagattgagagactcacagacacataaagagagag aaaagagggcagaggagtggtgagaatgatggcagggagcagagaaaagcactaaaattagagtcctgagagaga ggcacaaggacatagaaacatggagatgtggggatgaattgcagagattccaaagagagctagagagaccgagagg cagagcaatacagatgatagatggatagatatagatagatgataaataggtagatgatagataataggttaaagatacata gatgatgattgattgattcattaatagataatacatagagatgatgatgatgaagacagataatacgtacagatagagaggc agacagaaatcatagagagagagatgatacatacatataaataacagatgattgatggatagatagacaactgatagat acatagatgatatatagatatagatgacaggtagagaatttgtagataggcaccgaatagataaatagatagatcgacag ataatagatagaaatatgcagaaagttatgaacaggacacaacgtgagaaacttagaatttaaaaaagtaacatcaagtc aaccaatccaaggagagtcagagagaataaaacaatccaaaaacggaaaacatatctagaggtggggaagcgaggt cagagacctagagagacagagaaggtggaagaaggaaatagacatgaagagagatggggtggagggtgagagag agagagagagagcattaggtcatagagcaggggagtgagttctcagctcaggtgaagggagctgtgacaaggaagatc ctccctgaggaaaatgcctcttctccttccagGTCTATATGAGAAACCTTCTCTCTCAGCCCAGCCG GGCCCCACGGTTCTGGCAGGAGAGAGCGTGACCTTGTCCTGCAGCTCCCGGAGCT CCTATGACATGTACCATCTATCCAGGGAGGGGGAGGCCCATGAATGTAGGTTCTCT GCAGGGCCCAAGGTCAACGGAACATTCCAGGCCGACTTTCCTCTGGGCCCTGCCA CCCACGGAGGAACCTACAGATGCTTCGGCTCTTTCCGTGACTCTCCATACGAGTGG TCAAACTCGAGTGACCCACTGCTTGTTTCTGTCACAGgtgaggaaaccccatatctgtctcatgtc ctatgatcctagagccttagctgaggagcttcctgctgatgatggagataagcatggacagatgcagagagaagacgaag cttgggtgtgagggagggatcagggcacaggatggcagacagggcacctccaaaccctcctacacggcctgcatgaag gcccgcggccagggctccaggcacacaggcagatggagaaagcggtcaggagagacccagaggagggagactgg gctcagtttgggaagatcagaggttccctcagcccctcaacattacccatttcccagaagcccatcctggcctctcacccac acagggatgtcatcaccagcaacccctacaccctttacttttgtttgaagaaatatttattgaggataaatatacctatatagctt accacctttaacattttttttttttttgaggcagagtctagctctgtcccctatgctgcagtgcagtggcacaatctcagctcactgc aacttccgcctcctgggttcaagtgattctcctgcctcagccacctgagtagctggtgctacaggcgcgcaccaccacgcca ggctactttttgtatttttagtagagaggtggtttcaccatgttggtcgagctggtctccaactcctgaccacgtgatccacccgc atctgcctcccaaagtgctgggattacaggcatgagccaccactcccagccacatttaccatttttaagtgtaaagtctagtg gtcataaatacatttataaatatatatatatatatatatgtatgtatatatatatacacacacatatatatacatatatatatgtgtat atatatatatatatatatatatatatatatatatatatatttttttttttttttttaccctccacccttttcttcctggcctctggaagccacca ttctactctctaccttcatgagatccaccttttagctctgtatatgggtgagaaatgggaatctttgtaatgacttccagttccatcc atgtggctgcaaatatcaggatgttattctttctatggatgagtagtctccactgtgcgtatgtactacattctctctatccattcatc cactgatgggcaggtaggttgactccacatcttggctactgtgaacagtgctgcaccaatcatacgagtgcagatatcacttc gatatattgatttactttcctttggatataaacccagtagtgaaattgctggatactatgaaagttctctttttagttattcgtttgttgttt tgtttttgtttttgagacagtttccctctgtgcccaggctggagtacaagtgatgtcatcttggctcattgcaacctctgcctcctgg gttcaaatgattttcctacctcagcctccctagtagctgggattacaggtgcacgccaccatgcctggctactttttggtttttttag tatagatggggtttccccatgttggctgggctgctctcaaactcatgacctcaactgaggtgtccgcctcggtctcccaaagtg ccgggattacaggcatgatccacctcacccaacctctttttagttctttaaaggacttccacacttttctccgtaaaggctgtact aatttacactcctaccaacagggtattagggttctcctttctctaccactttggcaggatttcctttgcctgtcttgcagctaaaagc cattttactttatttcattttattttgagatggagtttcgctcttgtcacccaggctggagtgcagtggtgcgatctcggctcaccaca acctccacctcccaggttcaagcgattctcctgcctcagcctcccgagtagctggaattacaggcacacgccaccacgccc gactaatttttgtatttttagtagagacagtgtttctccatgtgggtcagactggtctcaaactcccgaccttatgagattcaccca cctcaggctctcaaagatctaggatgacagacgtgagccaccacgcccggcctaaaagccattttaatggggtgagatga aaactcactttgattttaatttgcgtttctctgatgatgagtgatactgagcagtttttcgtatgtggggaaatttcatgtcttttgctcct gtttcaattaaatcatttgttttattgagttgtttgagcttcttatatttctagttattaatcccatctcagatgcatagtttgcacatatttg ctcccaatctgtgggttgtctcttcactttgttggtttatttttagcggtgcagaagttgcttagcttgaggtaatcccaatggtctattt ttgcttcgattacttgtgttttgaaggtttaaaacaaaatgtcttccttcagacaaatgtcctggagcatttccccaatattttcttcta cgtgtttcataggttcaggccttagactcacatctttaatccattttcatttgatttttgtgtatggtgacaggtagaggtgcagtttca ttcctctgcatgtagatgtccaggtttccctgcactgtttattgaaaagactgtcctttcctgattgtgagttcttggcacctttgtcaa agtccattggatgggctgggcatggtgactgacacctgcaatttcagcactttgggagcccaaggcgggtggatcacctga ggccaggagttcaagattagtctggccgacgtgatgaaacattgtctccactaaaaatatataaattagctgagcatggtggt cagcacctataataccactactcaggagtttgaggccagagaattgattgaacccaggaggctgtggtggcagtgaaccg agattgcacctctgcactccagcctgggtgacagagcgagactccatctcaaaagaaaaaagaaaaaaacattggatgt aaatgcatggattatatttgtgttgttcattctgctccattgttctatgtgcctttcttcatgccaacatcatgctgtcttgcttactacag ctctgtaacatattttgagatcaggtagtgtgatgctcctgttttctctttataccttgaagtctcaagacaatgggcgtcacatac aaaaattatggaaaaaaggatcccaggactcccagggcccaatattagataacagagtgttggccatgaaccaacctca aagatttccattgagtagaggacagacaccctcatttcctcacctctctcctgtctcatgttctagGAAACCCTTCAAAT AGTTGGCCTTCACCCACTGAACCAAGCTCTAAAACCGgtgagtacagaaccctcttatatccgcttt tggaaacctggggaggtagaaaccttcgatgcaggcattgactcagcatctcgcagctctgacattgtacgcctgtcttctac catctccgaactccagatactccaacagcgaaagggatctgggcccaacctagggctcagtgaaatctcttaatctctcattt tatggagctgagacctcctacaagctagaagaatgattgccaatctgacatccttctcaggaaaaatgcaatgtttgttctgcc tgcattcctaactggaggataaattcctgggggcttgagagagggaagggaagggaacatctgatgagggcgaggtgtttt agagaagttccacttgccaaggaatgaattactgttggtcatgaagcaaccctggctgactcagcagagcaacagccttgc cgtaacagagaacggagctcatgcacgcacacttcgactcactgactcattcagccacggccccatgctcaggctgtgca gtgcggaaccttttcctattgttgccataacaaatttccacaagattcgtgggtgaaaaaaaacggttttttaattatcttacagt gctgtagctcaaagtaggaagtgcatcttactgggctaaaatcaaggtgacagcaaggctgccttccctctgaggattccag gcaagaatctgcttctcacttgtcccagcttctaaaggctcccagttccttggctcctggtccccttcctccttcctcaaaaccca caaagactggtcacatctcacatggcatcactcagtgccttcttccttaccacacctctttctctgaatgctgctctcccttcttcct tatcttttgaaaacttggggattctattgggttcaccaagatgaaaatccctcataatctcctggaaatcatccaggatacccttg ttttaagttcagctgattagcaaccgtaattccatctacaatcttcattcctcctttccatgtaaaataacatattcacaaggtatgg aggctaggacagggacattttggggtgggacagcattctcctgccttccacaaacagtgaacaagatgcatttggcctctgc ccttgggacactgatattgcagatggttaaatgggagggcagaaaatgaatgcacaagtggatctataaatgaatgatcca ttgggaagcatctgtgcatgaaatctattttttgtttgttcttttgtttattgagacagagttgccctctgtcttccaggctacagtgca gtgtcacgatcttggctcactgcaacctgcttctcctggattcaagtgattctcctgcctccgcctctcgagtagctgggattaca ggcaactgccaccgtgcccggctaattctttttgtatattttttgtagagaggatgtttcaccacgttggccaagcttgtctgaaac tcccaacctcaagtgatccgaccgtctcagcatgccaaagtaatgggactacaggcgtgagccactgtgcccagccaga attcaaaatcaataatagataatgctgagtgtatgatttcaggtgacaaagaaggtctcactattcagatatttgtgacattaat gaaaaacacggattgaacccctgaaagattggcggaaggattttgcacacacagctgtcagccgtgaaggcacaaagg tgaaaacaatctgatgtggaaggaagaggctcttcctcaaatgctgggaatgatgtggggagaatgacaagatgactgtg gagagacggagagcacactgggtacacaggaaactaaggaggaacaaggagtgtgtgtttgacactcacagccattgg attcacctcggggtagccaggaatccctacatgattaatatgactgacatgaaaataagggaggctcagttgcataactgg aatctaggagaccgtggaaaaggcaattgccgccccactggtgaaatgtggtgctgatttagacactaaatgaatgaagta gatggatataagataggtttgtgaggtagaatcattgactggaaaggcttgctgggtttgattttcctacttgtttaatcctcgctta attaatttctttctgagatttattcatcctacacataaatcaatacctggcaaaggagtgacagatatatgaggggtggtggaa atgaagagacctattatagcataatatacaagtctgtgaacggtggctcacgcctgtaacccagcactgcaggaggccaa ggcgggtggatcacatgaagtcagcagttcgagaccagcctggccaacatggtgaaaccctgtctctaggaaaaacaca aaaattagccgagcatggtggtgcatccctgtaatcccagctcctactctggaggatgaagcaggagaatgacttcaaccc aggaggtggaggttgcagtgagtggaggttgcatcactgcactccagcctgggtggcacaaggagactccgtctcaaaa aataaaaataagaaatgcataaatataaatataatataacacacgcaaatgacaaagggacctgaattccaatcatgattt ttctatttctctataattacttctttgatcctttatcttatccattaggcaatgagcctaaaacctcttccctatttggctttctgtgagcat gagatcatatagaaaatgtgaaagtccgctgaatcctccagcacagatcctggaatagagaaagtgctctggtcatcaca aaaaaaacttgcccactcacccaaatcccccacctcacccctacttccaatcacctgtggagattcaggtagaccatgggg aggtaaacattaacactccttggagtgagtccagatcttggaatcagagatcagcgacagcactagctcctgctcccctttcc tactaattcacaggaggacaggtggtattgaagcaatagatggccgagggggtggtccttcccccagcctctcgggtaga acagcagcctaatatgtgtctcccgagatcacaaagagcagcaggtttcacacgggcttcaacactatttcctggccgtttg acataagagaattctatttcgctttttttatcttgatttcacttttgttttctttccttggagaatgcaagttgtttgattcaagaatgctgt ggatgtagaaaccctaaagcacattcgctgtgaatcaatcccagtccagtcttcccagagaagactctaaacacctcctgg actgcacctgggcctatgccaattcctatcactcaccgtcactccagggagacagaacacacagagaatacgttacatag gcaggttcattactaacagataagcagcgagtgacaacagaaacctatatttcaatgtgagccagtccctcaaggctcaga aaagctcctcgggacatatggagtcaccccatttgcagtgtagctgcgggaagccagaaagcagcccagcctgggttttgt accctggagccacaggaagcactcagctaaagcactgcatgacgtcctccaggaagaacaggaagacagcccaggg tgttctgagacgttcctcctgatctcaggaagttgctgtcttaggccatttttgttgctctaaaggaacacttgagcctcggtaactt ctaaagaaaagagattggtttgcctcaccgttctgcaggctgtactggaagcatggcaccagcatctatttctcgtgacggcc tcaggctgctcccactctggcagaagggaaggagggtctgtctgtgcagagaccacagagatcacacggcaagagagg gagcaagggggagggggagtgatggagcttccaagctctttttaacaaccagctctccgggaactaatagagggggaac ttgctaaccccgtctccttgggacagcattgatgtgttcatgatggatccacctccatgacccaaacacctctcaagaggccc aacctcccacagtgggggtgaaatttcaatgtgaggtttgaaggggtcaaacatctcaactaaagtagtcgtatcctcagca cgttctatggttactatgagagctataactgaaaaagcaggagaaagctgggtctcctgccatctgggtgcttgtcctaaaga gatgttttatgtggttacctgtcaatcaagaaatgcgagacaattcataaagaggaactgctaagattagcttcttattggtgtct catcttcttccagGTAACCCCCGACACCTGCACATTCTGATTGGGACCTCAGTGGTCATCA TCCTCTTCATCCTCCTCTTCTTTCTCCTTCATCGCTGGTGCTCCAACAAAAAAAgtaag tctcacgaagcagaggccagagagctcagggccatgtggggaagcaggatgggagcactcaggtgtgtgttcctcaca aacaggatggtccctggcccaaggcagcagccacagaggcaggactttctagagagggcaccagactccctgcccctg ccttcaactcacagaccgttgcctgattctgaactgtatcctcatgtcccctgcagccactcacatccaggagaaggttccatg acaggcagaaagtgggagacagaatcaatgggatgggaactcagagctattcatgggatgggtccttgagctcagaga gatagaatgtctgagtctgctgttggcaactgagggacctcagccacctatggtctccccctgtatgttggtatctgcttatgaa atgaggacccagaagtgccctccgagctgttttgttgacttccgtctcctacagATGCTGCGGTAATGGACCAA GAGTCTGCAGGGAACAGAACAGCGAATAGCGAGgtaggtactcctcggcccgggctcgtggctact gttattcccaaagagtcctggaaaatgtgagcaccctccctcactcagcatttccctctctccagGACTCTGATGAAC AAGACCCTCAGGAGGTGACATACACACAGTTGAATCACTGCGTTTTCACACAGAGA AAAATCACTCGCCCTTCTCAGAGGCCCAAGACACCCCCAACAGATATCATCGTGTA CACGGAACTTCCAAATGCTGAGTCCAGATCCAAAGTTGTCTCCTGCCCATGAGCAC CACAGTCAGGCCTTGAGGGCGTCTTCTAGGGAGACAACAGCCCTGTCTCAAAAC CGGGTTGCCAGCTCCCATGTACCAGCAGCTGGAATCTGAAGGCATGAGTCTGCA TCTTAGGGCATCGCTCTTCCTCACACCACAAATCTGAATGTGCCTCTCACTTGCTT ACAAATGTCTAAGGTCCCCACTGCCTGCTGGAGAAAAAACACACTCCTTTGCTTA GCCCACAGTTCTCCATTTCACTTGACCCCTGCCCACCTCTCCAACCTAACTGGCTT ACTTCCTAGTCTACTTGAGGCTGCAATCACACTGAGGAACTCACAATTCCAAACA TACAAGAGGCTCCCTCTTAACGCAGCACTTAGACACGTGTTGTTCCACCTTCCCTC ATGCTGTTCCACCTCCCCTCAGACTAGCTTTCAGTCTTCTGTCAGCAGTAAAACTT ATATATTTTTTAAAATAACTTCAATGTAGTTTTCCATCCTTCAAATAAACATGTCTG CCCCCATG (SEQ ID NO:3), comprising a promoter region (italicized), 5′UTR and 3′UTR (upper case and bold), EXONS (upper case), and introns (lower case).

In some embodiments, the protein coding sequence of the KIR2DL2 gene has the consensus sequence: CGCGGCCGCCTGTCTGCACAGACAGCACCatgtcgctcatggtcgtcagcatggcgtgtgttgggttctt cttgctgcagggggcctggccaCATGAGGGAGTCCACAGAAAACCTTCCCTCCTGGCCCACC CAGGTCGCCTGGTGAAATCAGAAGAGACAGTCATCCTGCAATGTTGGTCAGATGTC AGGTTTGAGCACTTCCTTCTGCACAGAGAAGGGAAGTTTAAGGACACTTTGCACCT CATTGGAGAGCACCATGATGGGGTCTCCAAAGCCAACTTCTCCATCGGTCCCATGA TGCAAGACCTTGCAGGGACCTACAGATGCTACGGTTCTGTTACTCACTCCCCCTAT CAGTTGTCAGCTCCCAGTGACCCTCTGGACATCGTCATCACAGGTCTATATGAGAA ACCTTCTCTCTCAGCCCAGCCGGGCCCCACGGTTCTGGCAGGAGAGAGCGTGACC TTGTCCTGCAGCTCCCGGAGCTCCTATGACATGTACCATCTATCCAGGGAGGGGG AGGCCCATGAATGTAGGTTCTCTGCAGGGCCCAAGGTCAACGGAACATTCCAGGC CGACTTTCCTCTGGGCCCTGCCACCCACGGAGGAACCTACAGATGCTTCGGCTCTT TCCGTGACTCTCCATACGAGTGGTCAAACTCGAGTGACCCACTGCTTGTTTCTGTC ACAGGAAACCCTTCAAATAGTTGGCCTTCACCCACTGAACCAAGCTCTAAAACCGG TAACCCCCGACACCTGCACATTCTGATTGGGACCTCAGTGGTCATCATCCTCTTCAT CCTCCTCTTCTTTCTCCTTCATCGCTGGTGCTCCAACAAAAAAAATGCTGCGGTAAT GGACCAAGAGTCTGCAGGGAACAGAACAGCGAATAGCGAGGACTCTGATGAACAA GACCCTCAGGAGGTGACATACACACAGTTGAATCACTGCGTTTTCACACAGAGAAA AATCACTCGCCCTTCTCAGAGGCCCAAGACACCCCCAACAGATATCATCGTGTACA CGGAACTTCCAAATGCTGAGTCCAGATCCAAAGTTGTCTCCTGCCCATGAGCACCA CAGTCAGGCCTTGAGGGCGTCTTCTAGGGAGACAACAGCCCTGTCTCAAAACCG GGTTGCCAGCTCCCATGTACCAGCAGCTGGAATCTGAAGGCATGAGTCTGCATCT TAGGGCATCGCTCTTCCTCACACCACAAATCTGAATGTGCCTCTCACTTGCTTACA AATGTCTAAGGTCCCCACTGCCTGCTGGAGAAAAAACACACTCCTTTGCTTAGCC CACAGTTCTCCATTTCACTTGACCCCTGCCCACCTCTCCAACCTAACTGGCTTACT TCCTAGTCTACTTGAGGCTGCAATCACACTGAGGAACTCACAATTCCAAACATAC AAGAGGCTCCCTCTTAACGCAGCACTTAGACACGTGTTGTTCCACCTTCCCTCAT GCTGTTCCACCTCCCCTCAGACTAGCTTTCAGTCTTCTGTCAGCAGTAAAACTTAT ATATTTTTTAAAATAACTTCAATGTAGTTTTCCATCCTTCAAATAAACATGTCTGCC CCCATG (SEQ ID NO:3), comprising a 5′UTR and 3′UTR (upper case and bold) and a signal peptide (lower case).

In some embodiments, the protein coding sequence of the KIR2DL2 gene has the consensus sequence: CGCGGCCGCCTGTCTGCACAGACAGCACCatgtcgctcatggtcgtcagcatggcgtgtgttgggttctt cttgctgcagggggcctggccaCATGAGGGAGTCCACAGAAAACCTTCCCTCCTGGCCCACC CAGGTCGCCTGGTGAAATCAGAAGAGACAGTCATCCTGCAATGTTGGTCAGATGTC AGGTTTGAGCACTTCCTTCTGCACAGAGAAGGGAAGTTTAAGGACACTTTGCACCT CATTGGAGAGCACCATGATGGGGTCTCCAAAGCCAACTTCTCCATCGGTCCCATGA TGCAAGACCTTGCAGGGACCTACAGATGCTACGGTTCTGTTACTCACTCCCCCTAT CAGTTGTCAGCTCCCAGTGACCCTCTGGACATCGTCATCACAGGTCTATATGAGAA ACCTTCTCTCTCAGCCCAGCCGGGCCCCACGGTTCTGGCAGGAGAGAGCGTGACC TTGTCCTGCAGCTCCCGGAGCTCCTATGACATGTACCATCTATCCAGGGAGGGGG AGGCCCATGAATGTAGGTTCTCTGCAGGGCCCAAGGTCAACGGAACATTCCAGGC CGACTTTCCTCTGGGCCCTGCCACCCACGGAGGAACCTACAGATGCTTCGGCTCTT TCCGTGACTCTCCATACGAGTGGTCAAACTCGAGTGACCCACTGCTTGTTTCTGTC ACAGGAAACCCTTCAAATAGTTGGCCTTCACCCACTGAACCAAGCTCTAAAACCGG TAACCCCCGACACCTGCACATTCTGATTGGGACCTCAGTGGTCATCATCCTCTTCAT CCTCCTCTTCTTTCTCCTTCATCGCTGGTGCTCCAACAAAAAAAATGCTGCGGTAAT GGACCAAGAGTCTGCAGGGAACAGAACAGCGAATAGCGAGGACTCTGATGAACAA GACCCTCAGGAGGTGACATACACACAGTTGAATCACTGCGTTTTCACACAGAGAAA AATCACTCGCCCTTCTCAGAGGCCCAAGACACCCCCAACAGATATCATCGTGTACA CGGAACTTCCAAATGCTGAGTCCAGATCCAAAGTTGTCTCCTGCCCATGAGCACCA CAGTCAGGCCTTGAGGGCGTCTTCTAGGGAGACAACAGCCCTGTCTCAAAACCG GGTTGCCAGCTCCCATGTACCAGCAGCTGGAATCTGAAGGCATGAGTCTGCATCT TAGGGCATCGCTCTTCCTCACACCACAAATCTGAATGTGCCTCTCACTTGCTTACA AATGTCTAAGGTCCCCACTGCCTGCTGGAGAAAAAACACACTCCTTTGCTTAGCC CACAGTTCTCCATTTCACTTGACCCCTGCCCACCTCTCCAACCTAACTGGCTTACT TCCTAGTCTACTTGAGGCTGCAATCACACTGAGGAACTCACAATTCCAAACATAC AAGAGGCTCCCTCTTAACGCAGCACTTAGACACGTGTTGTTCCACCTTCCCTCAT GCTGTTCCACCTCCCCTCAGACTAGCTTTCAGTCTTCTGTCAGCAGTAAAACTTAT ATATTTTTTAAAATAACTTCAATGTAGTTTTCCATCCTTCAAATAAACATGTCTGCC CCCATG (SEQ ID NO:4), comprising a 5′UTR and 3′UTR (upper case and bold) and a signal peptide (lower case).

In some embodiments, the KIR2DL3 gene has the consensus sequence: gagcacccactgggcctcatgcaaggtagaaagagcctgcgtacgtcaccctcccatgatgtggtcaacatgtaaactgc atgggcagggcgccaaataacatcctgtgcgctgctgagctgagctGGGGCGCGGCCGCCTGTCTGCAC AGACAGCACCATGTCGCTCATGGTCGTCAGCATGGTGTGTGTTGgtgagtcctggaaggg catcgagggagggagtgcggggatggagatcggggcccagagttggagatataggcctggaagtggagttatgggcct agagatggagtgatgggcctagaagtggagatctgggcctggagtggagatctgggcctggagtggagatatgggcctg gaggttgagatatgggcctgcagtagagatatgggcttgtagtggagacatgggcctggagatggagatatgggcctgga gatggagatatgggcctgcagtagagataggggcctggagtggagatatgggcctggagtggagatatgggcctggagt ggagatatgggcctggaggtggagatatgggcctggaggtggagatatgggcctggagtggagatatgggtctggaggt ggagatacgggcctgcagtagagatatgggcctggagtggagatatgggccaggagtggagttatgggcctagaggtgg atatctgggcctggagtggagatatgggcctaggaaggagatatgggcctgggtgtggagatatgggactggagaggtg atatgggcctggagtggagatatgggcttagggtggagatctgggcctggggcggagatatgggactggattggagatag gggcctagggtggagatctgagcctggattggcgatatgggcctagggtggaaatatcagcctggagtggagatatgggc ttggggggggatatgggcctggaaactgggtctctgcacagccgacagccctgttcttgggtgcaggtaggcactgaggg tgagtttaacttcagcccaggaagggcctggctgccaagactcacagcccagtgggggcagcaagggaggcctggtttg cctgcagatggatggtccatcatgatctttctttccagGGTTCTTCTTGCTGCAGGGGGCCTGGCCACA TGAGGgtgagtccttctccaaaccttcgggtgtcatctccccacataagaggattttcctgaaacaggagggaagtcctgt cggggagtctctcataaactaggaagagaggaccctggggtgctcagcccacatttctgacctcgcctccctggcctctca accccttggcagagtcaagttctgtggggaccagggttagactggggtgctcaaagctggggtgtgtggttgggaagtggt aggaacagcagatcctctgaggacaaaggtgttactcacacacttcagcgtttccatgatggtaggggctgcagtgtggct gctgtcattctaccagaagaggtgggaaaccacagccatggccctgacattccaaatcctctgatgggggctcagttgtttat tttcgttcaggcatccgctgatatccattcacaaaggacatgccctccacctcatgtctaccctgtgttgttttatgtgagtaatctt acagtattaaaatctagtaggagtctctttactcagcacttgctcaaagttctcagctgaggcttttgttgtagggagacaccat gtctttgcgggatgggtccttccttcagccctgggcaccaaggtgtgatagtagccatagaaacgtggaaagcgaggaga atcttctgagcacagggagggaagggcagttccacatcctcctctctaaggcggcgcctccttctccccaaggtggtcagg acaagcccttgctgtctgcctggcccagccttgtggtgcctctaggacatgtcattcttcggtgtcactcttatcttgggtttaaca acttcagtctgtaaaaggaaggtggggtgcctgtccctgagctctacaacagaatattctggaacagccttttcatgggccct gtgacccccgcacacacagggacatacagatgtcggggttcacacacacactcccccagtgggtggtcagcacccagc aaccccctggtgatcgtggtcataggtcagagggctcctgtcttggattctccttgtcccacctcctgaatcccagagcttctgtt gggcatgtccttgagggtcccatcacgcaggccctgactgtatttgtggtaaagggggattgaatacagggaaatgggtgct gtggtgggaagaataattgtccccagtgatgactacattctaatccctggagtctgtgactatttatgttataggggaagggac tgaaggggaagatggagctcatggggagacagcctggactgtcccactgggctcagtgtaatcacaagggtgcacatga aaggaggaggaagaggggagtggggattagagcagtccagtggaagtcttcaccagctttgaaggggaggaaggcc aagatccatgaatgcaggtggcctatagaggctggaaaagtcaaggaactgattctccagagtctccagagggaacaaa gccctgcagatgccttgattttagcccaggaaaaatagggtccaatttctgtctccagtactggaaggtgtcagtgtggtctctc ctgctgccatgcttctgataattttctacagcagcaacaggaaaccaacactggaacccaggtcaaggacaagttaagaa acaacccaaggaaagccaggcatggtggcaggtgcatgtaatcctagcgactcaggaggctgagggcaggagaatca cttgaacccaggagacagaggttgcagtgagcctagaccacaccacttcactccagcctgggtgaaggagtgagactct gtctccataattaattaattaattaaagaaaccaaacaaggagaaggttggctaccctgagatcagcaaggggggatgat gatgccaccaccaggctccatccacatagggaggggttgatactcctccaaccagcaccaggagccagcctatggaag ctggcaccatggagaaggcacaggcatggcaagagtggctcccagtccccaccaggaacagggtgtgtggacactggt gcctgccttattcatcagttcataccttctgccaaggattgcaattcatccaaaagagattgaaccaggctgataagagcctg gatgtgcagcctatcctggttcctctttcacccccacataaacagcaggaaatacattagtgtgaaatagatacaacacccc aagagatgaggctcagcccagtgggaagggaatcagaggctactagagacagagggacagagaagagggaggga gacagatggaaggacctgcaccaggagttaagggcacagaaaagaacatgaagacacagagaggaaggagagag acagacaccagcaaggggaagcctcactcattctaggtgccatggatgggatgataaagagagacaccttctaaactca caacctctcttcctagGAGTCCACAGAAAACCTTCCCTCCTGGCCCACCCAGGTCCCCTGG TGAAATCAGAAGAGACAGTCATCCTGCAATGTTGGTCAGATGTCAGGTTTCAGCAC TTCCTTCTGCACAGAGAAGGGAAGTTTAAGGACACTTTGCACCTCATTGGAGAGCA CCATGATGGGGTCTCCAAGGCCAACTTCTCCATCGGTCCCATGATGCAAGACCTTG CAGGGACCTACAGATGCTACGGTTCTGTTACTCACTCCCCCTATCAGTTGTCAGCT CCCAGTGACCCTCTGGACATCGTCATCACAGgtgagagtgtccggacattctcattgtcattgggatgc agagtgaatgatccacgacttggaacccccaggtagttgtaaggaagatgagcttggtattcttatggagagagactgactt gctgaggtttgtaccaacagagacagagaaacaggagacacaagtacagaccaggtgtcataacagaggacagaca caggggccatacagggagttagaaaagacagaaagagttaaaagagacagacagacagacatgtcccagagagag gtgtccctccatgctgactttgctcacagacctggcacaggttagaagtttcatttctgttttacctccacaaagtgttctctacca ggagaacccaaggacacccatatttatgacctgagttgggccctgtggcctcaggccttgtggcacctacaggccatgttta ttctgacacctctgccttccatgtaatggagagtaatcgtcccaggatatcatggccccagaacaccaacccctgtatgctgt gtgaacttgtggtctccagactggattctgtggctcacattccaaataaccccacatatgaaaggatcactgagaggcacag agaaaaatcaggaacaccaaaaagcaaagacataaacacacagagaatgagccagaggaaggagattgagagac tcacagacacataaagagagagaaaagagggcagaggagtggtgagaatgatggcagggagcagagaaaagcact aaaattagagtcctgagagagaggcacaaggacatagaaacatggagatgtggggatgaattgcagagattccaaaga gaactagagagaccgagaggcagagcaagacagatgatagatggatagatatagatagatgataaataggtagatgat agataataggttaaagatacatagatgatgattgattgattcattaatagataatacatagagatgatgatgatgaagacaga taatacgtacagatagagaggcagacagaaatcatagagagagagatgatacatacatataaataacagatgattgatg gatagatagacaagtgatagatacatagatgatatatagatatagatgacaggtagagaatttgtagataggcaccgaata gataaatagatagatcgacagataatagatagaaatatgcagaaagttatgaacaggacacaacgtgagaaacttaga atttaaaaaagtaacatcaagtcaaccaatccaaggagagtcagagagaataaaagaatccaaaaagggaaaacata tctagaggtggggaagcgaggtcagagacctagagagacagagaaggtggaagaaggaaatagacatgaagagag atggggtggagggtgagagagagagagagagagagcattaggtcatagagcaggggagtgagttctcagctcaggtg aagggagctgtgacaaggaagatcctccgtaaggaaaatgcctcttctcctccagGTCTATATGAGAAACCTT CTCTCTCAGCCCAGCCGGGCCCCACGGTTCTGGCAGGAGAGAGCGTGACCTTGTC CTGCAGCTCCCGGAGCTCCTATGACATGTACCATCTATCCAGGGAGGGGGAGGCC CATGAACGTAGGTTCTCTGCAGGGCCCAAGGTCAACGGAACATTCCAGGCCGACT TTCCTCTGGGCCCTGCCACCCACGGAGGAACCTACAGATGCTTCGGCTCTTTCCGT GACTCTCCATACGAGTGGTCAAACTCGAGTGACCCACTGCTTGTTTCTGTCACAGgt gaggaaaccccatatctgtctcatgtcctatgatcctagagccttagctgaggagcttcctgctgatgatggagagaagcatg gacagatgcagagagaagacgaagcttgggtgtgagggagggatcagggcacaggatggcagacagggcacctcca aaccctcctacacggcctgcatgaaggcccgcggccagggctccaggcacacaggcagatggagaaaacggtcagg agagacccagaggagagagactgggctcagtttgggaagatcagaggttccctcagcccctcaacattacccatttccca gaagcccatcctggcctctcacccacacagggatgtcatcaccagcaacccctacaccctttacttttgtttgaagaaatattt attgaggataaatatacctatatagcttaccacctttaacattttttttttttttgaggcagagtctagctctgtcccctatgctggagt gcagtggcacaatctcagctcactgcaacttccgcctcctgggttcaagtgattctcctgcttcagccacctgagtagctggtg ctacaggcgcgcaccaccacgccaggctactttttgtatttttagtagagagggggtttcaccatgttggtcgagctggtctcc aactcctgaccacgtgatccacccgcatctgcctcccaaagtgctgggattacaggcatgagccaccacgcccagccac atttaccatttttaagtgtaaagtctagtggtcataaatacatttatatatatatatatatatatatatatacacacacacacacata tataaacatatatatatatatatatatatatatatatatatttttttttttttttttttttttaccctccacccttttattcctggcctctggaagc caccattctactctctaccttcatgagatccaccttttagctctgtatatgggtgagaaatgggaatctttgtaatgacttccagttc catccatgtggctgcaaatatcaggatgttattctttctatggatgagtagtctccactgtgcgtatgtactacattctctctatccat tcatccactgatgggcaggtaggttgactccacatcttggctactgtgaacagtgctgcaccaatcatacgagtgcagatatc acttcgatatattgatttactttcctttggatataaacccagtagtgaaattgctggatactatgaaagttctctttttagttattcgttt gttgttttgtttttgtttttgagacagtttccctctgtgcccaggctggagtacaagtgaagtcatcttggctcattgcaacctccgcc tcctgggttcaaatgattttcctgcctcagcctccctagtagctgggattacaggtgcacgccaccatgcctggctactttttgtttt ttttagtatagatggggtttccccatgttggctgggctgctctcaaactcatgacctcaactgaggtgcccgcctcggtctccca aagtgccgggattacaggcatgatccacctcacccaacctctttttagttctttaaaggacttccacacttttctccgtaaaggct gtactaatttacactcctaccaacagggtattagggttctcctttctctaccactttggcaggatttcctttgcctgtcttgcagctaa aagccattttattttatttcattttattttgagatggagtttcgctcttgtcacccaggctggagtgcagtggtgcgatctcggctcac cacaacctccacctcccaggttcaagcgattctcctgcctcagcctcccgagtagctggaattacaggcacacgccaccac gcccaactaaattttgtatttttagtagagacagtgtttcttcatgtgggtcagactggtctcaaactcccgaccttatgaggttca cccacctcaggctctcaaaggtctaggatgacagacgtgagccaccacgcccggcctaaaatccattttaatggggtgag atgaaaactcactttgattttaatttgtgtttctctgatgatgagtgaaactgagcactttttagtatgtggggaaatttcatgtgttttg ctcctttttcaattaaatcgtttgttttattgagttgtttgagcttcttatatttctagttattaatcccatctcagatgcatagtttgcacat atttgctcccaatctgtgggttgtctcttcactttgttggtttatttttagcggtgcagaagttgcttagtttgaggtaatcccaatggtc tatttttgcttcgattacttgtgttttgaaggtttaaaacaaaatgtcttccttcagacaaatgtcctggagcatttccccaatattttct tctacgtgtttcataggttcaggccttagactcacatctttaatccattttcatttgagttttgtgtatagtgacaggtagaggtgcag tttcattcctctgcatgtagatgtccaggtttccctgcactgtttattgaaaagactgtcctttcctgattgtgagttcttggcacctttg tcaaagtccattggatgggctgggcatggtggctgacacctgcaatttcagcactttgggagcccaaggcgggtggatcac ctgaggccaggagttcaagattagtctggccgacgtgatgaaacattgtctccactaaaaatataaaaattagctgagcatg gtggtcagcacctgtaataccactactcaggagtttgaggccagagaattgattgaacccaggaggctgtggtggcagtga accgagattgcacctctgcactccagcctgggtgacagagcgagactccatctcaaaagaaaaaagaaaaaaacattg gaggtaaatgcatggattatatctgtgttcttcattctgctccattgttctacgtgcctttctttatgccaatgtgatgctgttttgcttac tacagctctgtaacatattttgagatcaggtagtgtgatgctcctgttttctctttataccttgaagtctcaagacagtgggcgtca catacaaaaattacggaaaaaaggatcccaggactcccagggcccaatattagataacagagtgttggccatgaacca acctcaaagatttccattgagtagaggacagacaccctcatttcctcacctctctcctgtctcgtgttctagGAAACCCTT CAAATAGTTGGCCTTCACCCACTGAACCAAGCTCCGAAACCGgtgagtacagaaccctcttat atccgcttttggaaacctggggaggtagaaaccttcgatgcaggcattgactcagcatctcgcagctctgacattgtacgcct gtcttctaccatctccgaactccagatactccaacagcgaaagggatctgggcccaacctagggctcagtgaaatctcttaa tctctcattttatggagctgagacctcctacaagctagaagaatgattgccaatctgacatccttctcaggaaaaatgcaatgtt tgttctgcctgcattcctaactggaggataaattcctgggggcttgagagagggaagggaagggaacatctgatgagggc gaggtgttttagagaagttccacttgccaaggaatgaattactgttggtcatgaagcaaccctggctgactcagcagagcaa cagccttgccgtaacagagaacggagctcatgcacgcacacttcgactcactgactcattcagccacggccccatgctca ggctgtgcagtgcggaaccttttcctattgttgccataacaaatttccacaagattcgtgggtgaaaacaaaacggttttttaatt atcttacagtgctgtagctcaaagtaggaagtgcatcttactgggctaaaatcaaggtgacagcaaggctgccttccctctga ggattccaggcaagaatctgcttctcacttatcccagcttctaaaggctcccagttccttggctcctgttccccttcctccttcctc aaagcccacaaagactggtcacatctcacatggcatcactcagtgccttcttccttaccacacctotttctctgaatgctgctct cccttcttccttatcttttgaaaacttggggattctattgggttcaccaagatgaaaatccctcataatctcctggaaatcatccag gatacccttgttttaagttcagctgattagcaaccgcaattccatctacaatcttcattcctcctttccatgtaaaataacatattca caagctatggaggctaggacagggacattttgggggggacagcattctcctgccttccacaaacggtgaacaagatgca tttggcttctgcccttgggacactgatattgcagatggttaaatgggagggcagaaaatgaatgcacaagtggatctataaat gaatgatccattgggaagcatctgtgcatgaaatctattttttgtttgttcttttgtttattgagacagagtcgccctctgtcttccagg ctacagtgcagtgtcacgatcttggctcactgcaacctgcgtctcctggattcaagtgattctcctgcctccgcctctcgagtag ctgggattacaggcaactgccaccgtgcccggctaattctttttgtatattttttgtagagaggatgtttcaccacgttggccaag cttgtctgaaactcccaacctcaagtgatccgaccgtctcagcatgccaaagtaatgggactacaggcgtgagccactgtg cccagccagaattcaaaatcaataatagataatgctgagtgtatgatttcaggtgacaaagaaggtctcactattcagatattt gtgacattaatgaaaaacacggattgaacccctgaaagattggcggaaggattttgcacacacagctgtcagccgtgaag gcacaaaggtgaaaacaatctgatgtggaaggaagaggctctgcctcaaatgctgggaatgatgtggggagaatgaca agacgactgtagagagacggagagcacactgggtacacaggaaactaaggagcaacaaggagtgtgtgtttgacactc acagccattggattcacctcggggtaaccaggaatccctacatgattaatatgactgacatgaaaataagggaggctcagt tgcataactggaatctaggagaccgtggaaaaggcaattgccaccccactggtgaaatgtggtgctgatttagacactaaa tgaatgaagtagatggatataagatatgtttgtgaggtagaatcattgactggaaacgcttactgggtttgattttcctacttgttt aatcctcgcttaattaatttctttctgagatttattcatcctacacataaatcaatacctggcaaaggagtgacagatatatgagt ggtggtggaaatgaagagacttattatagcataatatacaagtctgtgaacagtggctcacgcctgtaacctagcactgcag gaggccaaggtgggtggattccatgaagtcaggagttccagaccagcctggccaacgtggtgaaaccctatctctactaa aaatacaaaaattagccgagcacgatggtgcatccctgtaatcccagctcctattctggaggatgaagcaggagaatgac ttcaacccagtaggtggaggttgcagtgagtggagattgcatcactgcactccagcctgggggacacaaggagactctatc tcaaaaaataaaaataagaaatacataaatataataaaacacacacgaatgacaaaggcacctgaattccaatcatcgtt tttctatttctctataattacttctttgatcctttatcttatccattaggcaatgagcttaaaacctcttccctatttggctttctgtgagaat gagatcacatagaaaatgtgaaagccctcagaatcctccagcacagatcgtggaatagagaaagtgctctgttcatcgca acaaaaaacttgcccactcacccaaatcccccacctcacccctacttccaatcacctgtggagattcagataggctatggg gaggtaaacattgatactccttggagtgagtccagatcttggaatcagagatcagtgccagcactagctcctgctcccctttc ctactaattcacaggaggacaggtggtattgaagcaatagatggccgagggggggtccttcccccagcctctcgggtaga acagcagcctaacatgtgtctcccgagatcacaaagagtagcacgtttcacacgggcttcaacactatttcctggccatttga cataagagaattctacttagctttttttatcttgatttcacttttgtttccttttcttggagaatgcaagttgtttgattcaagaatgctgtg gatgtagaaatcctaaagcacattcgctgtgtatcaatcccagtgcagtcttcccagagaagactctaaatacctcctggact gcacctgggcttatgccaattcctatcactcaccgtcactccagggagacagaacacacagagaatacattacacaggca ggttcattactaacagataagcagcgagtgacaacagaaacctacatttcaatgtgagccagtccctcaaggctcagaaa agctactcgggacatatggagtcaccccatttgcagtgtagctgggggaagccagagagcagcccagcctgggttttgtac tgtggagccacaggaagcactcagctaaagcactgcatgacgtcctcctccaggaagaacaggaagacagcccaggc tgttctgagacgttcctcctgatctcaggacgttgctgtcttagtccatttttgttgctctaaaggaacacttgagcctgggtaactt ctagagaaaagagattggtttgcctcacagttctgcaggctgtactggaagcgtggcaccagcatctatttctcgtgacggcc tcaggctgctcccactctggcagaagggaaggagggtctgtctgtgcagagaccacagagatcacacggcaagagagg gagcaagggggagggggagcgatggagcttccaagctcttttgaacaaccagctctccaggaactaatagaaggggaa cttgctaaccccgtctccttgggacagcattggtctgttcatgatggatccacctccatgacccaaacacctctcaagaggcc caacctcccacagtgggggtgaaatttcaatgtgaggtttgaaggggtcaaacatctcaactaaagtagttgtatcctcaac acgttctatggttactatgagagctataactgagaaagcaggagaaagctgggtctccctccatctgggtgcttgtcctaaag gggtgttgtatgtggttacctgtcaatcaagaaatgtgagacaattcataaagaggaactgctatgattagcttcttattggtgtc tcctcttcttccagGTAACCCCAGACACCTGCATGTTCTGATTGGGACCTCAGTGGTCATCA TCCTCTTCATCCTCCTCCTCTTCTTTCTCCTTCATCGCTGGTGCTGCAACAAAAAAAg taagtctcacgaagcagaggccagagagctcagggccatgtggggaagcaggatgggagcactcaggtgtgtgttcctc acagacaggatggtccctggcccaaggcagcagccacagagggaggactttctagagagagcaccagactccctgtcc ctgccttcagctcacagaccattgcctgattctgaactgtatcctcatgtcccctgcagccactcacatccaggagaaggttcc atgacaggcagaaagtgggagacagaatcaatgggatgggaactcagagctattcatgggatgggtccttgagctcaga gagatagaatgtctgagtctgctgttggcaactgagggacctcaggctcctatggtctccccctgtatgttggtatctgcttatga aatgagggcccagaagtgccctctgagctgttttgttgacttccgtcttctacagATGCTGTTGTAATGGACCAA GAGCCTGCAGGGAACAGAACAGTGAACAGGGAGgtaggtgctcctcggcccagcctcgtggctagt gttattcccaaagagtcctggaaaatgtgagcaccctccctcactcagcatttccctctctccagGACTCTGATGAAC AAGACCCTCAGGAGGTGACATATGCACAGTTGAATCACTGCGTTTTCACACAGAGA AAAATCACTCGCCCTTCTCAGAGGCCCAAGACACCCCCAACAGATATCATCGTGTA CACGGAACTTCCAAATGCTGAGCCCTGATCCAAAGTTGTCTCCTGCCCATGAGCA CCACAGTCAGGCCTTGAGGGGATCTTCTAGGGAGACAACAGCCCTGTCTCAAAA CTGGGTTGCCAGCTCCAATGTACCAGCAGCTGGAATCTGAAGGCGTGAGTCTGC ATCTTAGGGCATCGCTCTTCCTCACACCACAAATCTGAACGTGCCTCTCCCTTGCT TACAAATGTCTAAGGTCCCCACTGCCTGCTGGAGAGAAAACACACTCCTTTGCTT AGCCCACAATTCTCCATTTCACTTGACCCCTGCCCACCTCTCCAACCTAACTGGCT TACTTCCTAGTCTACTTGAGGCTGCAATCACACTGAGGAACTCACAATTCCAAAC ATACAAGAGGCTCCCTCTTAACACGGCACTTAGACACGTGCTGTTCCACCTTCCC TCATGCTGTTCCACCTCCCCTCAGACTAGCTTTCAGCCTTCTGTCAGCAGTAAAAC TTATATATTTTTTAAAATAATTTCAATGTAGTTTTCCCTCCTTCAAATAAACATGTCT GCCCTCA (SEQ ID NO:624), comprising a promoter region (italicized), 5′UTR and 3′UTR (upper case and bold), EXONS (upper case), and introns (lower case).

In some embodiments, the protein coding sequence of the KIR2DL3 gene has the consensus sequence: GGGGCGCGGCCGCCTGTCTGCACAGACAGCACC CATGAGGGAGTCCACAGAAAACCTTCCCTCCTGGC CCACCCAGGTCCCCTGGTGAAATCAGAAGAGACAGTCATCCTGCAATGTTGGTCAG ATGTCAGGTTTCAGCACTTCCTTCTGCACAGAGAAGGGAAGTTTAAGGACACTTTG CACCTCATTGGAGAGCACCATGATGGGGTCTCCAAGGCCAACTTCTCCATCGGTCC CATGATGCAAGACCTTGCAGGGACCTACAGATGCTACGGTTCTGTTACTCACTCCC CCTATCAGTTGTCAGCTCCCAGTGACCCTCTGGACATCGTCATCACAGGTCTATAT GAGAAACCTTCTCTCTCAGCCCAGCCGGGCCCCACGGTTCTGGCAGGAGAGAGCG TGACCTTGTCCTGCAGCTCCCGGAGCTCCTATGACATGTACCATCTATCCAGGGAG GGGGAGGCCCATGAACGTAGGTTCTCTGCAGGGCCCAAGGTCAACGGAACATTCC AGGCCGACTTTCCTCTGGGCCCTGCCACCCACGGAGGAACCTACAGATGCTTCGG CTCTTTCCGTGACTCTCCATACGAGTGGTCAAACTCGAGTGACCCACTGCTTGTTTC TGTCACAGGAAACCCTTCAAATAGTTGGCCTTCACCCACTGAACCAAGCTCCGAAA CCGGTAACCCCAGACACCTGCATGTTCTGATTGGGACCTCAGTGGTCATCATCCTC TTCATCCTCCTCCTCTTCTTTCTCCTTCATCGCTGGTGCTGCAACAAAAAAAATGCT GTTGTAATGGACCAAGAGCCTGCAGGGAACAGAACAGTGAACAGGGAGGACTCTG ATGAACAAGACCCTCAGGAGGTGACATATGCACAGTTGAATCACTGCGTTTTCACA CAGAGAAAAATCACTCGCCCTTCTCAGAGGCCCAAGACACCCCCAACAGATATCAT CGTGTACACGGAACTTCCAAATGCTGAGCCCTGATCCAAAGTTGTCTCCTGCCCAT GAGCACCACAGTCAGGCCTTGAGGGGATCTTCTAGGGAGACAACAGCCCTGTCT CAAAACTGGGTTGCCAGCTCCAATGTACCAGCAGCTGGAATCTGAAGGCGTGAG TCTGCATCTTAGGGCATCGCTCTTCCTCACACCACAAATCTGAACGTGCCTCTCCC TTGCTTACAAATGTCTAAGGTCCCCACTGCCTGCTGGAGAGAAAACACACTCCTT TGCTTAGCCCACAATTCTCCATTTCACTTGACCCCTGCCCACCTCTCCAACCTAAC TGGCTTACTTCCTAGTCTACTTGAGGCTGCAATCACACTGAGGAACTCACAATTCC AAACATACAAGAGGCTCCCTCTTAACACGGCACTTAGACACGTGCTGTTCCACCT TCCCTCATGCTGTTCCACCTCCCCTCAGACTAGCTTTCAGCCTTCTGTCAGCAGTA AAACTTATATATTTTTTAAAATAATTTCAATGTAGTTTTCCCTCCTTCAAATAAACAT GTCTGCCCTCA (SEQ ID NO:625), comprising a 5′UTR and 3′UTR (upper case and bold) and a signal peptide (lower case).

In some embodiments, KIR2DL3 has the amino acid sequence: mslmvvsmvcvgffllqgawpHEGVHRKPSLLAHPGPLVKSEETVILQCWSDVRFQHFLLHRE GKFKDTLHLIGEHHDGVSKANFSIGPMMQDLAGTYRCYGSVTHSPYQLSAPSDPLDIVI TGLYEKPSLSAQPGPTVLAGESVTLSCSSRSSYDMYHLSREGEAHERRFSAGPKVNG TFQADFPLGPATHGGTYRCFGSFRDSPYEWSNSSDPLLVSVTgnpsnswpsptepssetgnp rhlhvligtsvviilfilllfflIHRWCCNKKNAVVMDQEPAGNRTVNREDSDEQDPQEVTYAQLN HCVFTQRKITRPSQRPKTPPTDIIVYTELPNAEP (SEQ ID NO:626, NP_056952.2), comprising a signal peptide (lower case and italicized), EXTRACELLULAR DOMAIN (capitalized), stem region (lower case), transmembrane domain (lower case and bold, and INTRACELLULAR DOMAIN (capitalized and bold).

Therefore, disclosed herein are gRNAs and crRNAs that can be used to ablate KIR2DL3 expression in immune effector cells. Note that differences between a gRNA and a crRNA are based on Cas specificities. While gRNAs are designed to couple with a tracer RNA (tracRNA) to pair with the Cas9, the crRNA are designed to form a ribonucleoprotein (RNP) complex with the Cas12 with no need of tracRNA.

In some embodiments, the gRNA and crRNA can be used to ablate KIR2DL2 and/or KIR2DL3 expression in immune effector cells are provided in Table 1 and/or 2.

TABLE 1
KIR2DL2 guide RNA (gRNA*) and CRISPR RNA (crRNA*)
Cas9

| | | | | Specificity | Efficiency |
|---|---|---|---|---|---|
| | | | | Score | Score |
| Region | | | | (Off target | (On target |
| targeted | Strand | Sequence | PAM | score) | score) |
| Promoter | 1 | ctcttgagcgagcacccact (SEQ ID NO: 5) | ggg | 15.9815022 | 46.26123025 |
| | 1 | gctcttgagcgagcacccac (SEQ ID NO: 6) | tgg | 15.9325168 | 38.82200489 |
| | −1 | agtgggtgctcgctcaagag (SEQ ID NO: 7) | cgg | 15.8645005 | 61.65603932 |
| | −1 | gctcgctcaagagcggaaca (SEQ ID NO: 8) | tgg | 15.6348821 | 59.891163 |
| | −1 | taccttgcatgaggcccagt (SEQ ID NO: 9) | ggg | 15.3838426 | 47.99589073 |
| | 1 | tggtcaacatgtaaactgca (SEQ ID NO: 10) | tgg | 14.8022274 | 60.25262163 |
| | 1 | tgtgcgctgctgagctgagc (SEQ ID NO: 11) | tgg | 14.58646 | 26.51616174 |
| | 1 | ggtcaacatgtaaactgcat (SEQ ID NO: 12) | ggg | 14.1942045 | 66.69398072 |
| | −1 | catgggagggtgacgtacgc (SEQ ID NO: 13) | agg | 14.1725166 | 59.57214176 |
| | −1 | atgttgaccacatcatggga (SEQ ID NO: 14) | ggg | 13.9805565 | 67.58548647 |
| | −1 | tttacatgttgaccacatca (SEQ ID NO: 15) | tgg | 13.9640537 | 60.21717187 |
| | 1 | acatgtaaactgcatgggca (SEQ ID NO: 16) | ggg | 13.8728239 | 59.4772864 |
| | −1 | gcagcgcacaggatgttatt (SEQ ID NO: 17) | tgg | 13.8464645 | 38.09327197 |
| | 1 | tgcgctgctgagctgagctg (SEQ ID NO: 18) | ggg | 13.7588893 | 52.71880216 |
| | 1 | aacatgtaaactgcatgggc (SEQ ID NO: 19) | agg | 13.4718305 | 46.46066649 |
| | −1 | agctcagctcagcagcgcac (SEQ ID NO: 20) | agg | 13.2454872 | 48.30097161 |
| | −1 | ttacatgttgaccacatcat (SEQ ID NO: 21) | ggg | 12.7849142 | 58.65244492 |
| | 1 | gtgcgctgctgagctgagct (SEQ ID NO: 22) | ggg | 12.0932581 | 29.66669834 |
| | 1 | tacgtcaccctcccatgatg (SEQ ID NO: 23) | tgg | 12.0547498 | 57.3970135 |
| | −1 | catgttgaccacatcatggg (SEQ ID NO: 24) | agg | 12.0445433 | 60.83312873 |
| | 1 | ggctctttctaccttgcatg (SEQ ID NO: 25) | agg | 11.895067 | 56.77308491 |
| | −1 | ctaccttgcatgaggcccag (SEQ ID NO: 26) | tgg | 11.6270243 | 63.92002251 |
| | 1 | cacccactgggcctcatgca (SEQ ID NO: 27) | agg | 11.622461 | 55.11316701 |
| 5′UTR + | 1 | agacagcaccatgtcgctca (SEQ ID NO: 28) | tgg | 65.7857251 | 61.74680156 |
| Signal | −1 | atggtgctgtctgtgcagac (SEQ ID NO: 29) | agg | 50.7755911 | 61.01765228 |
| peptide | −1 | gtgctgtctgtgcagacagg (SEQ ID NO: 30) | cgg | 42.4198704 | 65.8510863 |
| | −1 | gctgacgaccatgagcgaca (SEQ ID NO: 31) | tgg | 17.4180397 | 68.97222201 |
| | 1 | gtcgctcatggtcgtcagca (SEQ ID NO: 32) | tgg | 15.8075075 | 48.88926217 |
| Signal | 1 | cagggggcctggccacatga (SEQ ID NO: 33) | ggg | 25.6759978 | 53.42004189 |
| peptide + | 1 | ggactcaccctcatgtggcc (SEQ ID NO: 34) | agg | 24.7318646 | 50.06679646 |
| Exon 2 | 1 | cagggttcttcttgctgcag (SEQ ID NO: 35) | ggg | 16.7082395 | 47.66879684 |
| | 1 | ccagggttcttcttgctgca (SEQ ID NO: 36) | ggg | 16.5530134 | 42.85183717 |
| | 1 | tccagggttcttcttgctgc (SEQ ID NO: 37) | agg | 16.4294081 | 25.66544124 |
| | 1 | tcttcttgctgcagggggcc (SEQ ID NO: 38) | tgg | 16.3937902 | 23.97461357 |
| | 1 | agggttcttcttgctgcagg (SEQ ID NO: 39) | ggg | 16.3044063 | 53.09881316 |
| | 1 | gcagggggcctggccacatg (SEQ ID NO: 40) | agg | 15.8202174 | 47.71767808 |
| | 1 | ccctgcagcaagaagaaccc (SEQ ID NO: 41) | tgg | 15.2116359 | 54.97024219 |
| Region | | | | Specificity | Efficiency |
| targeted | Strand | Sequence | PAM | Score | Score |
| Exon 3 | −1 | cctgcaaggtcttgcatcat (SEQ ID NO: 42) | ggg | 79.5879626 | 41.94420172 |
| | 1 | cccatgatgcaagaccttgc (SEQ ID NO: 43) | agg | 72.4619282 | 47.76943418 |
| | −1 | ccctgcaaggtcttgcatca (SEQ ID NO: 44) | tgg | 72.4330928 | 50.76857508 |
| | 1 | ccatgatgcaagaccttgca (SEQ ID NO: 45) | ggg | 68.7469834 | 60.94960431 |
| | 1 | aaggacactttgcacctcat (SEQ ID NO: 46) | tgg | 67.2663317 | 49.30177556 |
| | −1 | gtcttgcatcatgggaccga (SEQ ID NO: 47) | tgg | 54.8096904 | 54.75667126 |
| | 1 | gcacagagaagggaagttta (SEQ ID NO: 48) | agg | 48.9411384 | 37.28340475 |
| | −1 | accgatggagaagttggctt (SEQ ID NO: 49) | tgg | 40.5434549 | 35.55658063 |
| | −1 | tctgatttcaccaggcgacc (SEQ ID NO: 50) | tgg | 36.9267672 | 39.34508609 |
| | −1 | ctgtgatgacgatgtccaga (SEQ ID NO: 51) | ggg | 36.8255359 | 64.83990028 |
| | −1 | catcatggtgctctccaatg (SEQ ID NO: 52) | agg | 31.0596287 | 67.52356313 |
| | 1 | cctggcccacccaggtcgcc (SEQ ID NO: 53) | tgg | 30.3422942 | 40.4554791 |
| | 1 | tgcaatgttggtcagatgtc (SEQ ID NO: 54) | agg | 28.852938 | 42.43970536 |
| | 1 | ctgatttcaccaggcgacct (SEQ ID NO: 55) | ggg | 28.2209907 | 50.80004796 |
| | 1 | tcattggagagcaccatgat (SEQ ID NO: 56) | ggg | 25.536319 | 54.0081352 |
| | 1 | ctcattggagagcaccatga (SEQ ID NO: 57) | tgg | 24.7282726 | 54.6636991 |
| | 1 | cattggagagcaccatgatg (SEQ ID NO: 58) | ggg | 22.3248805 | 64.51568239 |
| | −1 | tttcaccaggcgacctgggt (SEQ ID NO: 59) | ggg | 21.945129 | 56.88886502 |
| | −1 | atttcaccaggcgacctggg (SEQ ID NO: 60) | tgg | 21.3848745 | 59.80225741 |
| | 1 | tccaaagccaacttctccat (SEQ ID NO: 61) | cgg | 20.3315154 | 54.61351716 |
| | 1 | acttccttctgcacagagaa (SEQ ID NO: 62) | ggg | 20.0787861 | 54.50715496 |
| | −1 | acttcccttctctgtgcaga (SEQ ID NO: 63) | agg | 19.0230959 | 53.39949127 |
| | −1 | ccaggcgacctgggtgggcc (SEQ ID NO: 64) | agg | 18.1730721 | 41.63264924 |
| | −1 | gacgatgtccagagggtcac (SEQ ID NO: 65) | tgg | 17.8540881 | 39.43995673 |
| | −1 | tggctttggagaccccatca (SEQ ID NO: 66) | tgg | 17.6135951 | 57.28898297 |
| | −1 | agcatctgtaggtccctgca (SEQ ID NO: 67) | agg | 17.5270305 | 61.14244139 |
| | −1 | catgggaccgatggagaagt (SEQ ID NO: 68) | tgg | 17.5075974 | 47.77644971 |
| | −1 | gggagctgacaactgatagg (SEQ ID NO: 69) | ggg | 17.1716987 | 66.56681924 |
| | −1 | ctgtctcttctgatttcacc (SEQ ID NO: 70) | agg | 17.0018113 | 52.48561839 |
| | −1 | ctgggagctgacaactgata (SEQ ID NO: 71) | ggg | 16.7225486 | 44.13169675 |
| | −1 | tgggagctgacaactgatag (SEQ ID NO: 72) | ggg | 16.6426956 | 58.90662026 |
| | −1 | aacagaaccgtagcatctgt (SEQ ID NO: 73) | agg | 15.5732951 | 66.08963393 |
| | −1 | actgggagctgacaactgat (SEQ ID NO: 74) | agg | 14.7844691 | 50.36680662 |
| | 1 | gtcagctcccagtgaccctc (SEQ ID NO: 75) | tgg | 13.9887563 | 42.25362939 |
| | −1 | acgatgtccagagggtcact (SEQ ID NO: 76) | ggg | 13.7921026 | 60.75050183 |
| | −1 | ggcgacctgggtgggccagg (SEQ ID NO: 77) | agg | 13.6590717 | 57.639565 |
| | 1 | gcgacctgggtgggccagga (SEQ ID NO: 78) | ggg | 13.2100431 | 50.05340313 |
| | −1 | tgacatctgaccaacattgc (SEQ ID NO: 79) | agg | 12.8591707 | 41.43631776 |
| | 1 | gcagggacctacagatgcta (SEQ ID NO: 80) | cgg | 12.7977995 | 59.98223405 |
| | 1 | agacagtcatcctgcaatgt (SEQ ID NO: 81) | tgg | 12.6543101 | 51.27120381 |
| | 1 | cacttccttctgcacagaga (SEQ ID NO: 82) | agg | 12.553479 | 47.24932091 |
| | 1 | ccacagaaaaccttccctcc (SEQ ID NO: 83) | tgg | 12.179199 | 51.892235 |
| | 1 | ccttccctcctggcccaccc (SEQ ID NO: 84) | agg | 11.4972374 | 53.63053228 |
| | −1 | ccaggagggaaggttttctg (SEQ ID NO: 85) | tgg | 11.2593255 | 54.0490957 |
| | −1 | cctgggtgggccaggaggga (SEQ ID NO: 86) | agg | 10.6308453 | 45.10008683 |

Cas9

| | | | | Specificity | Efficiency |
|---|---|---|---|---|---|
| | | | | Score | Score |
| Region | | | | (Off target | (On target |
| targeted | Strand | Sequence | PAM | score) | score) |
| Exon 4 | −1 | ctgcagagaacctacattca (SEQ ID NO: 87) | tgg | 58.126023 | 41.77042991 |
| | 1 | agggggaggcccatgaatgt (SEQ ID NO: 88) | agg | 57.5984019 | 54.91868055 |
| | −1 | tgcagagaacctacattcat (SEQ ID NO: 89) | ggg | 53.8049699 | 47.1931636 |
| | 1 | catgaatgtaggttctctgc (SEQ ID NO: 90) | agg | 48.8037638 | 47.15855912 |
| | 1 | atgaatgtaggttctctgca (SEQ ID NO: 91) | ggg | 42.9541779 | 63.70794223 |
| | 1 | tccgtgactctccatacgag (SEQ ID NO: 92) | tgg | 42.3518877 | 64.95530799 |
| | −1 | gctctctcctgccagaaccg (SEQ ID NO: 93) | tgg | 39.9122374 | 53.48535523 |
| | −1 | agcatctgtaggttcctccg (SEQ ID NO: 94) | tgg | 32.9490905 | 60.90621662 |
| | 1 | taggttctctgcagggccca (SEQ ID NO: 95) | agg | 31.8896396 | 56.55116007 |
| | −1 | ctcgagtttgaccactcgta (SEQ ID NO: 96) | tgg | 31.0199135 | 56.19377776 |
| | −1 | accactcgtatggagagtca (SEQ ID NO: 97) | cgg | 29.8382482 | 67.75748604 |
| | −1 | ctctctcctgccagaaccgt (SEQ ID NO: 98) | ggg | 28.317125 | 40.78289164 |
| | −1 | ggcccagaggaaagtcggcc (SEQ ID NO: 99) | tgg | 27.118728 | 35.48158427 |
| | −1 | ctggaatgttccgttgacct (SEQ ID NO: 100) | tgg | 26.6891718 | 39.22415654 |
| | −1 | tggaatgttccgttgacctt (SEQ ID NO: 101) | ggg | 26.2257382 | 43.51741671 |
| | 1 | tctgcagggcccaaggtcaa (SEQ ID NO: 102) | cgg | 25.5217031 | 58.68215106 |
| | −1 | gcatctgtaggttcctccgt (SEQ ID NO: 103) | ggg | 23.7641164 | 64.09139455 |
| | −1 | ggatagatggtacatgtcat (SEQ ID NO: 104) | agg | 22.5719786 | 58.79313198 |
| | 1 | caaggtcaacggaacattcc (SEQ ID NO: 105) | agg | 22.5410529 | 45.79626069 |
| | 1 | ccgggccccacggttctggc (SEQ ID NO: 106) | agg | 22.1712727 | 46.55746509 |
| | 1 | atgacatgtaccatctatcc (SEQ ID NO: 107) | agg | 22.0305575 | 44.75201749 |
| | −1 | tctctcctgccagaaccgtg (SEQ ID NO: 108) | ggg | 20.8300787 | 67.22642591 |
| | −1 | ttcatgggcctccccctccc (SEQ ID NO: 109) | tgg | 20.4729882 | 39.96596612 |
| | 1 | tgtaccatctatccagggag (SEQ ID NO: 110) | ggg | 20.034197 | 58.18580496 |
| | −1 | ataggagctccgggagctgc (SEQ ID NO: 111) | agg | 18.8988426 | 49.6265444 |
| | 1 | attccaggccgactttcctc (SEQ ID NO: 112) | tgg | 18.831583 | 31.78584288 |
| | −1 | taggttcctccgtgggtggc (SEQ ID NO: 113) | agg | 18.6401946 | 33.90580348 |
| | 1 | ttccaggccgactttcctct (SEQ ID NO: 114) | ggg | 18.3037756 | 51.86413845 |
| | 1 | atgtaccatctatccaggga (SEQ ID NO: 115) | ggg | 17.9123846 | 65.85919053 |
| | 1 | tgacatgtaccatctatcca (SEQ ID NO: 116) | ggg | 17.6685576 | 64.17145284 |
| | −1 | ccagaaccgtggggcccggc (SEQ ID NO: 117) | tgg | 17.5233952 | 26.25436916 |
| | −1 | cctgccagaaccgtggggcc (SEQ ID NO: 118) | cgg | 17.4086492 | 46.57815807 |
| | −1 | cagaaccgtggggcccggct (SEQ ID NO: 119) | ggg | 17.2023021 | 40.47921169 |
| | 1 | ggaggaacctacagatgctt (SEQ ID NO: 120) | cgg | 17.1023595 | 52.37081136 |
| | 1 | ccagccgggccccacggttc (SEQ ID NO: 121) | tgg | 16.4944144 | 31.16041254 |
| | 1 | gtaccatctatccagggagg (SEQ ID NO: 122) | ggg | 16.2348456 | 59.07845567 |
| | - | gtacatgtcataggagctcc (SEQ ID NO: 123) | ggg | 15.844385 | 54.15898367 |
| | 1 | catgtaccatctatccaggg (SEQ ID NO: 124) | agg | 15.7311139 | 71.77109475 |
| | 1 | ccatctatccagggaggggg (SEQ ID NO: 125) | agg | 15.6443179 | 45.35911519 |
| | −1 | ggtacatgtcataggagctc (SEQ ID NO: 126) | cgg | 15.5683706 | 46.55891931 |
| | −1 | ggcagggcccagaggaaagt (SEQ ID NO: 127) | cgg | 15.4697357 | 49.6486146 |
| | −1 | cctccccctccctggataga (SEQ ID NO: 128) | tgg | 15.4331486 | 37.06286031 |
| | −1 | tctgtaggttcctccgtggg (SEQ ID NO: 129) | tgg | 14.5487998 | 63.6725736 |
| | −1 | ctgtgacagaaacaagcagt (SEQ ID NO: 130) | ggg | 14.167692 | 70.81083421 |
| | −1 | gctccgggagctgcaggaca (SEQ ID NO: 131) | agg | 13.2586199 | 56.31871721 |
| | 1 | tgaccttgtcctgcagctcc (SEQ ID NO: 132) | cgg | 13.1335213 | 44.62611872 |
| | −1 | aggttcctccgtgggtggca (SEQ ID NO: 133) | ggg | 12.996601 | 51.90925589 |
| | 1 | ctcagcccagccgggcccca (SEQ ID NO: 134) | cgg | 12.6112714 | 45.24662974 |
| | 1 | ctgggccctgccacccacgg (SEQ ID NO: 135) | agg | 12.2718516 | 60.36188811 |
| | 1 | ccttctctctcagcccagcc (SEQ ID NO: 136) | ggg | 12.0416884 | 50.40044061 |
| | −1 | gaaagagccgaagcatctgt (SEQ ID NO: 137) | agg | 11.8256907 | 64.81372513 |
| | 1 | accttctctctcagcccagc (SEQ ID NO: 138) | cgg | 11.5867593 | 40.49696239 |
| | −1 | cccggctgggctgagagaga (SEQ ID NO: 139) | agg | 10.6934148 | 48.09196456 |
| | −1 | ccgtgggtggcagggcccag (SEQ ID NO: 140) | agg | 10.0379832 | 59.97182871 |
| | 1 | cctctgggccctgccaccca (SEQ ID NO: 141) | cgg | 9.6833026 | 49.04004993 |
| Exon 5 | −1 | tactcaccggttttagagct (SEQ ID NO: 142) | tgg | 57.852457 | 35.49280109 |
| | 1 | actgaaccaagctctaaaac (SEQ ID NO: 143) | cgg | 48.7345562 | 54.98912793 |
| | −1 | gttttagagcttggttcagt (SEQ ID NO: 144) | ggg | 37.0092113 | 55.32743764 |
| | −1 | ggttttagagcttggttcag (SEQ ID NO: 145) | tgg | 36.4002409 | 55.47355384 |
| | −1 | ggtgaaggccaactatttga (SEQ ID NO: 146) | agg | 20.7173391 | 43.55714515 |
| | 1 | taggaaacccttcaaatagt (SEQ ID NO: 147) | tgg | 20.5263725 | 42.8174115 |
| | −1 | gtgaaggccaactatttgaa (SEQ ID NO: 148) | ggg | 19.9184363 | 51.3711373 |
| | −1 | gagcttggttcagtgggtga (SEQ ID NO: 149) | agg | 14.748832 | 55.63346666 |
| Exon 6 | −1 | tcagaatgtgcaggtgtcgg (SEQ ID NO: 150) | ggg | 58.3772674 | 58.97985736 |
| | −1 | atcagaatgtgcaggtgtcg (SEQ ID NO: 151) | ggg | 55.9818205 | 54.73894422 |
| | 1 | cgacacctgcacattctgat (SEQ ID NO: 152) | tgg | 41.7051956 | 45.76563799 |
| | −1 | tgcaggtgtcgggggttacc (SEQ ID NO: 153) | tgg | 41.1213682 | 41.27410693 |
| | −1 | caatcagaatgtgcaggtgt (SEQ ID NO: 154) | cgg | 38.0757244 | 53.47960664 |
| | −1 | aatcagaatgtgcaggtgtc (SEQ ID NO: 155) | ggg | 36.8116094 | 41.65166937 |
| | −1 | aggtcccaatcagaatgtgc (SEQ ID NO: 156) | agg | 35.8407269 | 60.52618338 |
| | 1 | gacacctgcacattctgatt (SEQ ID NO: 157) | ggg | 31.2544152 | 39.39880007 |
| | −1 | tgttggagcaccagcgatga (SEQ ID NO: 158) | agg | 22.967965 | 50.79660146 |
| | 1 | cattctgattgggacctcag (SEQ ID NO: 159) | tgg | 21.2419253 | 67.83334055 |
| | −1 | gaagaggatgatgaccactg (SEQ ID NO: 160) | agg | 18.7676197 | 77.02928766 |
| | −1 | gtgagacttactttttttgt (SEQ ID NO: 161) | tgg | 17.8896759 | 35.81269774 |
| | 1 | tcttctttctccttcatcgc (SEQ ID NO: 162) | tgg | 16.0977219 | 41.84089734 |
| | −1 | gaaagaagaggaggatgaag (SEQ ID NO: 163) | agg | 14.9946626 | 56.87278435 |
| | −1 | agcgatgaaggagaaagaag (SEQ ID NO: 164) | agg | 12.9288675 | 58.06787307 |
| | −1 | gatgaaggagaaagaagagg (SEQ ID NO: 165) | agg | 9.0025407 | 66.1476459 |
| Exon 7 | 1 | tcctacagatgctgcggtaa (SEQ ID NO: 166) | tgg | 39.0868305 | 42.2970887 |
| | −1 | tccattaccgcagcatctgt (SEQ ID NO: 167) | agg | 38.9014202 | 58.3166372 |
| | 1 | taatggaccaagagtctgca (SEQ ID NO: 168) | ggg | 36.3621703 | 63.23445862 |
| | 1 | gaacagaacagcgaatagcg (SEQ ID NO: 169) | agg | 32.2502225 | 67.08996298 |
| | 1 | agaacagcgaatagcgaggt (SEQ ID NO: 170) | agg | 31.2359366 | 63.3723398 |
| | 1 | gtaatggaccaagagtctgc (SEQ ID NO: 171) | agg | 29.6230987 | 56.94666444 |
| | −1 | ttctgttccctgcagactct (SEQ ID NO: 172) | tgg | 25.0386945 | 36.77304996 |

Cas9

| Region | | | | Specificity | Efficiency |
|---|---|---|---|---|---|
| targeted | Strand | Sequence | PAM | Score | Score |
| Exon 8 | 1 | aacagatatcatcgtgtaca (SEQ ID NO: 173) | cgg | 45.2890654 | 62.71699919 |
| 3′ UTR | −1 | gtacacgatgatatctgttg (SEQ ID NO: 174) | ggg | 44.9529219 | 61.98329108 |
| | 1 | gtgtacacgatgatatctgt (SEQ ID NO: 175) | tgg | 44.2078237 | 57.17566505 |
| | −1 | tgtacacgatgatatctgtt (SEQ ID NO: 176) | ggg | 43.5868555 | 42.60355046 |
| | −1 | tacacgatgatatctgttgg (SEQ ID NO: 177) | ggg | 43.2709289 | 72.67309996 |
| | −1 | ttggatctggactcagcatt (SEQ ID NO: 178) | tgg | 26.7862203 | 36.78849828 |
| | −1 | tgtgtgtatgtcacctcctg (SEQ ID NO: 179) | agg | 26.5688904 | 56.3411972 |
| | −1 | gtgtgtatgtcacctcctga (SEQ ID NO: 180) | ggg | 26.0070899 | 61.3014034 |
| | −1 | gcaggagacaactttggatc (SEQ ID NO: 181) | tgg | 18.3112446 | 44.99853999 |
| | −1 | tcatgggcaggagacaactt (SEQ ID NO: 182) | tgg | 18.2856344 | 58.0617284 |
| | −1 | atatctgttgggggtgtctt (SEQ \|D NO: 183) | ggg | 17.9587785 | 34.80286512 |
| | −1 | gatatctgttgggggtgtct (SEQ ID NO: 184) | tgg | 17.5950576 | 33.17325643 |
| | −1 | ggtcttgttcatcagagtcc (SEQ ID NO: 185) | tgg | 14.7091383 | 51.61055238 |
| | 1 | ctctgatgaacaagaccctc (SEQ ID NO: 186) | agg | 14.5402588 | 50.15205354 |
| | 1 | aaatcactcgcccttctcag (SEQ ID NO: 187) | agg | 14.2544365 | 65.66875202 |
| | −1 | gtgtcttgggcctctgagaa (SEQ ID NO: 188) | ggg | 13.9295958 | 49.88141773 |
| | 1 | tgatgaacaagaccctcagg (SEQ ID NO: 189) | agg | 13.7606138 | 68.32294117 |
| | −1 | ggtgtcttgggcctctgaga (SEQ ID NO: 190) | agg | 13.7195437 | 37.36705244 |
| | −1 | gtctaagtgctgcgttaaga (SEQ ID NO: 191) | ggg | 84.6859457 | 48.63417996 |
| | −1 | tgtctaagtgctgcgttaag (SEQ ID NO: 192) | agg | 81.7777316 | 45.91914405 |
| | −1 | gagggagcctcttgtatgtt (SEQ ID NO: 193) | tgg | 76.8782493 | 35.37362392 |
| | 1 | gcccacctctccaacctaac (SEQ ID NO: 194) | tgg | 76.2988801 | 39.87036309 |
| | −1 | attgcagcctcaagtagact (SEQ ID NO: 195) | agg | 72.4829698 | 57.0478991 |
| | −1 | gactaggaagtaagccagtt (SEQ ID NO: 196) | agg | 72.2442137 | 46.00607944 |
| | −1 | aggaagtaagccagttaggt (SEQ ID NO: 197) | tgg | 71.9042489 | 41.7340746 |
| | −1 | gtaagccagttaggttggag (SEQ ID NO: 198) | agg | 71.54855 | 55.56736427 |
| | −1 | agactgaaagctagtctgag (SEQ ID NO: 199) | ggg | 69.6237996 | 65.42780553 |
| | 1 | cttacttcctagtctacttg (SEQ ID NO: 200) | agg | 67.8417065 | 55.35650233 |
| | −1 | gaagactgaaagctagtctg (SEQ ID NO: 201) | agg | 66.7107553 | 60.44061624 |
| | −1 | ctccctagaagacgccctca (SEQ ID NO: 202) | agg | 66.5546099 | 51.7559014 |
| | −1 | ctgaaagctagtctgagggg (SEQ ID NO: 203) | agg | 61.702876 | 67.32684796 |
| | 1 | ttgaggctgcaatcacactg (SEQ ID NO: 204) | agg | 61.31892 | 74.74770129 |
| | −1 | aagactgaaagctagtctga (SEQ ID NO: 205) | ggg | 60.6602522 | 54.56675355 |
| | 1 | gccagttaggttggagaggt (SEQ ID NO: 206) | ggg | 59.3258049 | 46.75048202 |
| | 1 | aggccttgagggcgtcttct (SEQ ID NO: 207) | agg | 58.5006215 | 28.99177389 |
| | −1 | tgggcaggggtcaagtgaaa (SEQ ID NO: 208) | tgg | 56.9295509 | 35.45301848 |
| | 1 | ggccttgagggcgtcttcta (SEQ ID NO: 209) | ggg | 56.5940243 | 41.23536534 |
| | 1 | cacaattccaaacatacaag (SEQ ID NO: 210) | agg | 55.9615864 | 61.51772376 |
| | −1 | ggggaggtggaacagcatga (SEQ ID NO: 211) | ggg | 54.6131044 | 69.50227076 |
| | −1 | aggggaggtggaacagcatg (SEQ ID NO: 212) | agg | 51.8017596 | 53.59962361 |
| | 1 | agccagttaggttggagagg (SEQ ID NO: 213) | tgg | 49.6070841 | 51.45946584 |
| | −1 | aaagctagtctgaggggagg (SEQ ID NO: 214) | tgg | 48.5212124 | 48.81089317 |
| | −1 | aggtggaacagcatgaggga (SEQ ID NO: 215) | agg | 47.5710532 | 63.15679382 |
| | −1 | tcaagtgaaatggagaactg (SEQ ID NO: 216) | tgg | 45.6822743 | 66.22020553 |
| | −1 | caagtgaaatggagaactgt (SEQ ID NO: 217) | ggg | 42.2396466 | 58.27515972 |
| | 1 | gttaggttggagaggtgggc (SEQ ID NO: 218) | agg | 42.1263881 | 37.29893803 |
| | −1 | tggaacagcatgagggaagg (SEQ ID NO: 219) | tgg | 41.6545549 | 56.47796615 |
| | −1 | acgccctcaaggcctgactg (SEQ ID NO: 220) | tgg | 35.5835131 | 65.38459652 |
| | 1 | gggcagacatgtttatttga (SEQ ID NO: 221) | agg | 33.9230448 | 36.96875759 |
| | 1 | taggttggagaggtgggcag (SEQ ID NO: 222) | ggg | 33.4801222 | 57.42383612 |
| | −1 | agacatgtttatttgaagga (SEQ ID NO: 223) | tgg | 30.5506105 | 52.39159675 |
| | −1 | gagtgtgttttttctccagc (SEQ ID NO: 224) | agg | 29.2922982 | 50.35103693 |
| | −1 | ttaggttggagaggtgggca (SEQ ID NO: 225) | ggg | 28.4425748 | 47.83669021 |
| | −1 | gacatttgtaagcaagtgag (SEQ ID NO: 226) | agg | 26.9419189 | 59.5267819 |
| | −1 | acattcagatttgtggtgtg (SEQ ID NO: 227) | agg | 26.4256821 | 59.99750155 |
| | −1 | gagaggcacattcagatttg (SEQ ID NO: 228) | tgg | 23.7410105 | 49.60496574 |
| | 1 | aaggcatgagtctgcatctt (SEQ ID NO: 229) | agg | 22.5261758 | 31.64102817 |
| | −1 | gttttttctccagcaggcag (SEQ ID NO: 230) | tgg | 21.269244 | 46.35960697 |
| | −1 | gagaactgtgggctaagcaa (SEQ ID NO: 231) | agg | 20.1230219 | 57.13606536 |
| | 1 | aggcatgagtctgcatctta (SEQ ID NO: 232) | ggg | 18.8279913 | 45.29561932 |
| | −1 | ggcctgactgtggtgctcat (SEQ ID NO: 233) | ggg | 18.0906072 | 46.09773639 |
| | −1 | agattccagctgctggtaca (SEQ ID NO: 234) | tgg | 18.0603432 | 49.56795823 |
| | −1 | gtacatgggagctggcaacc (SEQ ID NO: 235) | cgg | 17.8858383 | 63.6982707 |
| | 1 | cacttgcttacaaatgtcta (SEQ ID NO: 236) | agg | 16.9204605 | 48.62217882 |
| | −1 | agctgctggtacatgggagc (SEQ ID NO: 237) | tgg | 16.819902 | 45.14789672 |
| | 1 | agcaccacagtcaggccttg (SEQ ID NO: 238) | agg | 16.725956 | 60.15631648 |
| | 1 | ctaaggtccccactgcctgc (SEQ ID NO: 239) | tgg | 16.4850162 | 46.35050595 |
| | −1 | ttttttctccagcaggcagt (SEQ ID NO: 240) | ggg | 16.3504127 | 43.18290306 |
| | −1 | aggcctgactgtggtgctca (SEQ ID NO: 241) | tgg | 15.9475718 | 39.75667041 |
| | 1 | agctcccatgtaccagcagc (SEQ ID NO: 242) | tgg | 15.7399196 | 43.24124596 |
| | −1 | ggcaacccggttttgagaca (SEQ ID NO: 243) | ggg | 15.2164422 | 53.50908449 |
| | −1 | tttttctccagcaggcagtg (SEQ ID NO: 244) | ggg | 15.0293785 | 66.12065141 |
| | −1 | tggcaacccggttttgagac (SEQ ID NO: 245) | agg | 14.9169303 | 43.26076066 |
| | 1 | caacagccctgtctcaaaac (SEQ ID NO: 246) | cgg | 14.6632947 | 41.10299921 |
| | 1 | gcaccacagtcaggccttga (SEQ ID NO: 247) | ggg | 14.4012919 | 54.47726453 |
| | 1 | aacagccctgtctcaaaacc (SEQ ID NO: 248) | ggg | 14.0824143 | 61.19510127 |
| | −1 | gattccagctgctggtacat (SEQ ID NO: 249) | ggg | 12.8461481 | 42.84337761 |
| | −1 | tgccttcagattccagctgc (SEQ ID NO: 250) | tgg | 12.6088296 | 43.1813332 |
| | 1 | taccagcagctggaatctga (SEQ ID NO: 251) | agg | 11.2821164 | 58.57564638 |

Cpf1 (Cas12)

| Region | | | | Specificity | Efficiency |
|---|---|---|---|---|---|
| targeted | Strand | Sequence | PAM | Score | Score |
| KIR2DL2 | −1 | taccttgcatgaggcccagtg (SEQ ID NO: 252) | tttc | 12.4068985 | |
| promoter | −1 | gcgccctgcccatgcagttta (SEQ ID NO: 254) | ttta | 13.282476 | |
| Signal | −1 | catgttgaccacatcatggga (SEQ ID NO: 253) | tttg | 13.26842 | |
| peptide | 1 | cagggttcttcttgctgcagg (SEQ ID NO: 255) | tttc | 17.9146425 | |
| + Exon 2 | 1 | tttccagggttcttcttgctg (SEQ ID NO: 256) | tttc | 16.1254393 | |
| Exon 3 | −1 | gagaccccatcatggtgctct (SEQ ID NO: 257) | tttg | 96.1936289 | |
| | 1 | aggacactttgcacctcattg (SEQ ID NO: 258) | ttta | 85.2555973 | |
| | 1 | cacctcattggagagcaccat (SEQ ID NO: 259) | tttg | 38.7284003 | |
| | −1 | accaggcgacctgggtgggcc (SEQ ID NO: 260) | tttc | 24.7183139 | |
| | 1 | agcacttccttctgcacagag (SEQ ID NO: 261) | tttg | 17.5260664 | |
| Exon 4 | −1 | accactcgtatggagagtcac (SEQ ID NO: 262) | tttg | 39.5758627 | |
| | 1 | cgtgactctccatacgagtgg (SEQ ID NO: 263) | tttc | 39.261553 | |
| | 1 | ctctgggccctgccacccacg (SEQ ID NO: 264) | tttc | 16.4322184 | |
| Exon 5 | −1 | agagcttggttcagtgggtga (SEQ ID NO: 265) | tttt | 38.9033407 | |
| | −1 | gagcttggttcagtgggtgaa (SEQ ID NO: 266) | ttta | 22.0523188 | |
| Exon 6 | −1 | gttggagcaccagcgatgaag (SEQ ID NO: 267) | tttt | 33.1804647 | |
| | −1 | ttggagcaccagcgatgaagg (SEQ ID NO: 268) | tttg | 33.1493374 | |
| | −1 | tgttggagcaccagcgatgaa (SEQ ID NO: 269) | tttt | 33.0688783 | |
| | −1 | tttgttggagcaccagcgatg (SEQ ID NO: 270) | tttt | 33.0291427 | |
| | −1 | ttgttggagcaccagcgatga (SEQ ID NO: 271) | tttt | 28.3779427 | |
| | 1 | tccttcatcgctggtgctcca (SEQ ID NO: 272) | tttc | 22.0003974 | |
| Exon 8 | −1 | gaagttccgtgtacacgatga (SEQ ID NO: 273) | tttg | 33.0685405 | |
| | −1 | gatctggactcagcatttgga (SEQ ID NO: 274) | tttg | 32.3627985 | |
| | −1 | tctgtgtgaaaacgcagtgat (SEQ ID NO: 275) | tttc | 15.1976376 | |
| | −1 | ctctgtgtgaaaacgcagtga (SEQ ID NO: 276) | tttt | 15.1682898 | |
| | −1 | tctctgtgtgaaaacgcagtg (SEQ ID NO: 277) | tttt | 15.1501405 | |
| | 1 | acacagagaaaaatcactcgc (SEQ ID NO: 278) | tttc | 14.9951438 | |
| | 1 | cacacagagaaaaatcactcg (SEQ ID NO: 279) | tttt | 14.7463876 | |
| 3′ UTR | 1 | acttgacccctgcccacctct (SEQ ID NO: 280) | tttc | 94.5211894 | |
| | −1 | ctccagcaggcagtggggacc (SEQ ID NO: 281) | tttt | 92.6667631 | |
| | −1 | gaattgtgagttcctcagtgt (SEQ ID NO: 282) | tttg | 91.0259628 | |
| | −1 | actgctgacagaagactgaaa (SEQ ID NO: 283) | tttt | 88.8907291 | |
| | −1 | tccagcaggcagtggggacct (SEQ ID NO: 284) | tttc | 86.4077082 | |
| | 1 | agtcttctgtcagcagtaaaa (SEQ ID NO: 285) | tttc | 86.2360603 | |
| | −1 | ctgctgacagaagactgaaag (SEQ ID NO: 286) | ttta | 85.6577309 | |
| | −1 | taagcaagtgagaggcacatt (SEQ ID NO: 287) | tttg | 61.6642353 | |
| | −1 | aaggatggaaaactacattga (SEQ ID NO: 288) | tttg | 59.4762322 | |
| | −1 | tttgaaggatggaaaactaca (SEQ ID NO: 289) | ttta | 59.4515509 | |
| | 1 | ccatccttcaaataaacatgt (SEQ ID NO: 290) | tttt | 57.7176326 | |
| | 1 | catccttcaaataaacatgtc (SEQ ID NO: 291) | tttc | 55.7785529 | |
| | 1 | ttaaaataacttcaatgtagt (SEQ ID NO: 292) | tttt | 55.0105568 | |
| | 1 | aaataacttcaatgtagtttt (SEQ ID NO: 293) | ttta | 53.5669934 | |
| | 1 | taaaataacttcaatgtagtt (SEQ ID NO: 294) | tttt | 51.3980311 | |
| | 1 | aaaataacttcaatgtagttt (SEQ ID NO: 295) | tttt | 51.0928346 | |
| | −1 | aaaaaatatataagttttact (SEQ ID NO: 296) | tttt | 37.2680576 | |
| | −1 | ttctccagcaggcagtgggga (SEQ ID NO: 297) | tttt | 32.1407638 | |
| | −1 | aaaaatatataagttttactg (SEQ ID NO: 298) | ttta | 31.0317963 | |
| | −1 | tggtgtgaggaagagcgatgc (SEQ ID NO: 299) | tttg | 28.1189674 | |
| | 1 | cttagcccacagttctccatt (SEQ ID NO: 300) | tttg | 27.8217365 | |
| | −1 | tctccagcaggcagtggggac (SEQ ID NO: 301) | tttt | 16.3318587 | |
| | −1 | gagacagggctgttgtctccc (SEQ ID NO: 302) | tttt | 15.2323502 | |
| | −1 | agacagggctgttgtctccct (SEQ ID NO: 303) | tttg | 13.2493833 | |

TABLE 2
KIR2DL3 guide RNA (gRNA*) and CRISPR RNA (crRNA*) in silico analysis.
Cas9

| | | | | Specificity | Efficiency | |
|---|---|---|---|---|---|---|
| | | | | Score | Score | |
| Region | | | | (Off target | (On target | Targets |
| targeted | Strand | Sequence | PAM | score) | score) | KIR2DL2 |
| Promoter | −1 | taccttgcatgaggcccagt (SEQ ID NO: 304) | ggg | 15.3838426 | 47.99589073 | Yes |
| | 1 | tggtcaacatgtaaactgca (SEQ ID NO: 305) | tgg | 14.8022274 | 60.25262163 | Yes |
| | 1 | tgtgcgctgctgagctgagc (SEQ ID NO: 306) | tgg | 14.58646 | 26.51616174 | Yes |
| | 1 | ggtcaacatgtaaactgcat (SEQ ID NO: 307) | ggg | 14.1942045 | 66.69398072 | Yes |
| | −1 | catgggagggtgacgtacgc (SEQ ID NO: 308) | agg | 14.1725166 | 59.57214176 | Yes |
| | 1 | atgttgaccacatcatggga (SEQ ID NO: 309) | ggg | 13.9805565 | 67.58548647 | Yes |
| | −1 | tttacatgttgaccacatca (SEQ ID NO: 310) | tgg | 13.9640537 | 60.21717187 | Yes |
| | 1 | acatgtaaactgcatgggca (SEQ ID NO: 311) | ggg | 13.8728239 | 59.4772864 | Yes |
| | −1 | gcagcgcacaggatgttatt (SEQ ID NO: 312) | tgg | 13.8464645 | 38.09327197 | Yes |
| | 1 | tgcgctgctgagctgagctg (SEQ ID NO: 313) | ggg | 13.7588893 | 52.71880216 | Yes |
| | 1 | aacatgtaaactgcatgggc (SEQ ID NO: 314) | agg | 13.4718305 | 46.46066649 | Yes |
| | 1 | agctcagctcagcagcgcac (SEQ ID NO: 315) | agg | 13.2454872 | 48.30097161 | Yes |
| | −1 | ttacatgttgaccacatcat (SEQ ID NO: 316) | ggg | 12.7849142 | 58.65244492 | Yes |
| | 1 | gtgcgctgctgagctgagct (SEQ ID NO: 317) | ggg | 12.0932581 | 29.66669834 | Yes |
| | 1 | tacgtcaccctcccatgatg (SEQ ID NO: 318) | tgg | 12.0547498 | 57.3970135 | Yes |
| | −1 | catgttgaccacatcatggg (SEQ ID NO: 319) | agg | 12.0445433 | 60.83312873 | Yes |
| | 1 | ggctctttctaccttgcatg (SEQ ID NO: 320) | agg | 11.895067 | 56.77308491 | Yes |
| | −1 | ctaccttgcatgaggcccag (SEQ ID NO: 321) | tgg | 11.6270243 | 63.92002251 | Yes |
| | 1 | cacccactgggcctcatgca (SEQ ID NO: 322) | agg | 11.622461 | 55.11316701 | Yes |
| 5′UTR + | 1 | gctgacgaccatgagcgaca (SEQ ID NO: 323) | tgg | 17.4180397 | 68.97222201 | Yes |
| Signal | 1 | gtgctgtctgtgcagacagg (SEQ ID NO: 324) | cgg | 42.4198704 | 65.8510863 | Yes |
| peptide | 1 | agacagcaccatgtcgctca (SEQ ID NO: 325) | tgg | 65.7857251 | 61.74680156 | Yes |
| | 1 | atggtgctgtctgtgcagac (SEQ ID NO: 326) | agg | 50.7755911 | 61.01765228 | Yes |
| | 1 | gtcgtcagcatggtgtgtgt (SEQ ID NO: 327) | tgg | 45.1363023 | 58.61939694 | Partially, |
| | | | | | | mismatched |
| | 1 | gtcgctcatggtcgtcagca (SEQ ID NO: 328) | tgg | 15.8075075 | 48.38517422 | Yes |
| Signal | 1 | cagggggcctggccacatga (SEQ ID NO: 329) | ggg | 25.6759978 | 53.42004189 | Yes |
| peptide + | 1 | cagggttcttcttgctgcag (SEQ ID NO: 330) | ggg | 16.7082395 | 47.66879684 | Yes |
| Exon 2 | 1 | tcttcttgctgcagggggcc (SEQ ID NO: 331) | tgg | 16.3937902 | 23.97461357 | Yes |
| | 1 | agggttcttcttgctgcagg (SEQ ID NO: 332) | ggg | 16.3044063 | 53.09881316 | Yes |
| | 1 | gcagggggcctggccacatg (SEQ ID NO: 333) | agg | 15.8202174 | 47.71767808 | Yes |
| Exon 3 | −1 | cctgcaaggtcttgcatcat (SEQ ID NO: 334) | ggg | 79.5879626 | 41.94420172 | Yes |
| | 1 | cccatgatgcaagaccttgc (SEQ ID NO: 335) | agg | 72.4619282 | 47.76943418 | Yes |
| | −1 | ccctgcaaggtcttgcatca (SEQ ID NO: 336) | tgg | 72.4330928 | 50.76857508 | Yes |
| | 1 | ccatgatgcaagaccttgca (SEQ ID NO: 337) | ggg | 68.7469834 | 60.94960431 | Yes |
| | 1 | aaggacactttgcacctcat (SEQ ID NO: 338) | tgg | 67.2663317 | 49.30177556 | Yes |
| | 1 | gtcttgcatcatgggaccga (SEQ ID NO: 339) | tgg | 54.8096904 | 54.75667126 | Yes |
| | 1 | gcacagagaagggaagttta (SEQ ID NO: 340) | agg | 48.9411384 | 37.28340475 | Yes |
| | 1 | cctctggacatcgtcatcac (SEQ ID NO: 341) | agg | 44.5805237 | 55.04179415 | Yes |
| | −1 | ctgtgatgacgatgtccaga (SEQ ID NO: 342) | ggg | 36.8255359 | 64.83990028 | Yes |
| | −1 | catcatggtgctctccaatg (SEQ ID NO: 343) | agg | 31.0596287 | 67.52356313 | Yes |
| | −1 | cctgtgatgacgatgtccag (SEQ ID NO: 344) | agg | 30.9824377 | 71.65427178 | Yes |
| | 1 | tgcaatgttggtcagatgtc (SEQ ID NO: 345) | agg | 28.852938 | 42.43970536 | Yes |
| | 1 | tcatcacaggtgagagtgtc (SEQ ID NO: 346) | cgg | 26.6905717 | 49.76842395 | Yes |
| | 1 | tcattggagagcaccatgat (SEQ ID NO: 347) | ggg | 25.536319 | 54.0081352 | Yes |
| | 1 | ctcattggagagcaccatga (SEQ ID NO: 348) | tgg | 24.7282726 | 54.6636991 | Yes |
| | 1 | gcaccatgatggggtctcca (SEQ ID NO: 349) | agg | 24.1475235 | 58.30931536 | Yes |
| | −1 | tgtctcttctgatttcacca (SEQ ID NO: 350) | ggg | 23.9896226 | 63.57815515 | Yes |
| | 1 | cattggagagcaccatgatg (SEQ ID NO: 351) | ggg | 22.3248805 | 64.51568239 | Yes |
| | −1 | gtctcttctgatttcaccag (SEQ ID NO: 352) | ggg | 20.9813594 | 59.56514127 | Yes |
| | 1 | acttccttctgcacagagaa (SEQ ID NO: 353) | ggg | 20.0787861 | 55.07594659 | Yes |
| | −1 | acttcccttctctgtgcaga (SEQ ID NO: 354) | agg | 19.0230959 | 53.39949127 | Yes |
| | −1 | gacgatgtccagagggtcac (SEQ ID NO: 355) | tgg | 17.8540881 | 39.43995673 | Yes |
| | −1 | agcatctgtaggtccctgca (SEQ ID NO: 356) | agg | 17.5270305 | 61.14244139 | Yes |
| | 1 | catgggaccgatggagaagt (SEQ ID NO: 357) | tgg | 17.5075974 | 47.77644971 | Yes |
| | 1 | gggagctgacaactgatagg (SEQ ID NO: 358) | ggg | 17.1716987 | 66.56681924 | Yes |
| | −1 | ctgtctcttctgatttcacc (SEQ ID NO: 359) | agg | 17.0018113 | 47.05530645 | Yes |
| | −1 | tctgatttcaccaggggacc (SEQ ID NO: 360) | tgg | 16.9775981 | 32.03493004 | Partially, |
| | | | | | | mismatched |
| | −1 | ctgggagctgacaactgata (SEQ ID NO: 361) | ggg | 16.7225486 | 44.13169675 | Yes |
| | −1 | tgggagctgacaactgatag (SEQ ID NO: 362) | ggg | 16.6426956 | 58.90662026 | Yes |
| | −1 | ctgatttcaccaggggacct (SEQ ID NO: 363) | ggg | 16.4147201 | 49.6599218 | Partially, |
| | | | | | | mismatched |
| | −1 | tttcaccaggggacctgggt (SEQ ID NO: 364) | ggg | 15.7911939 | 51.66670072 | Partially, |
| | | | | | | mismatched |
| | 1 | aacagaaccgtagcatctgt (SEQ ID NO: 365) | agg | 15.5732951 | 66.08963393 | Yes |
| | −1 | atttcaccaggggacctggg (SEQ ID NO: 366) | tgg | 15.0670995 | 53.24259585 | Partially, |
| | | | | | | mismatched |
| | −1 | aaggttttctgtggactcct (SEQ ID NO: 367) | agg | 14.8391394 | 55.10050834 | Yes |
| | −1 | actgggagctgacaactgat (SEQ ID NO: 368) | agg | 14.7844691 | 50.36680662 | Yes |
| | −1 | tggccttggagaccccatca (SEQ ID NO: 369) | tgg | 14.7167449 | 61.35246116 | Partially, |
| | | | | | | mismatched |
| | 1 | cctggcccacccaggtcccc (SEQ ID NO: 370) | tgg | 14.4159475 | 37.66084916 | Partially, |
| | | | | | | mismatched |
| | −1 | accgatggagaagttggcct (SEQ ID NO: 371) | tgg | 14.1813168 | 41.93333267 | Partially, |
| | | | | | | mismatched |
| | 1 | gtcagctcccagtgaccctc (SEQ ID NO: 372) | tgg | 13.9887563 | 42.25362939 | Yes |
| | −1 | acgatgtccagagggtcact (SEQ ID NO: 373) | ggg | 13.7921026 | 60.75050183 | Yes |
| | 1 | tccaaggccaacttctccat (SEQ ID NO: 374) | cgg | 13.7074722 | 51.21442994 | Partially, |
| | | | | | | mismatched |
| | −1 | ccaggggacctgggtgggcc (SEQ ID NO: 375) | agg | 12.9401132 | 35.16922372 | Partially, |
| | | | | | | mismatched |
| | 1 | tgacatctgaccaacattgc (SEQ ID NO: 376) | agg | 12.8591707 | 41.43631776 | Yes |
| | 1 | gcagggacctacagatgcta (SEQ ID NO: 377) | cgg | 12.7977995 | 59.98223405 | Yes |
| | 1 | agacagtcatcctgcaatgt (SEQ ID NO: 378) | tgg | 12.6543101 | 51.27120381 | Yes |
| | 1 | cacttccttctgcacagaga (SEQ ID NO: 379) | agg | 12.553479 | 47.81811254 | Yes |
| | 1 | ccacagaaaaccttccctcc (SEQ ID NO: 380) | tgg | 12.179199 | 51.892235 | Yes |
| | 1 | ccttccctcctggcccaccc (SEQ ID NO: 381) | agg | 11.4972374 | 51.59938375 | Yes |
| | 1 | ccaggagggaaggttttctg (SEQ ID NO: 382) | tgg | 11.2593255 | 54.0490957 | Yes |
| | −1 | gggacctgggtgggccagga (SEQ ID NO: 383) | ggg | 11.1741438 | 48.26174904 | Partially, |
| | | | | | | mismatched |
| | 1 | cctgggtgggccaggaggga (SEQ ID NO: 384) | agg | 10.6308453 | 39.68873187 | Yes |
| | −1 | ggggacctgggtgggccagg (SEQ ID NO: 385) | agg | 9.4460011 | 42.78256738 | Partially, |
| | | | | | | mismatched |
| Region | | | | Specificity | Efficiency | Targets |
| targeted | Strand | Sequence | PAM | Score | Score | KIR2DL2 |

Cas9

| | | | | | | |
|---|---|---|---|---|---|---|
| Exon 4 | −1 | tgcagagaacctacgttcat (SEQ ID NO: 386) | ggg | 45.3001727 | 46.6010886 | Partially, |
| | | | | | | mismatched |
| | 1 | catgaacgtaggttctctgc (SEQ ID NO: 387) | agg | 43.9495309 | 50.00423578 | Partially, |
| | | | | | | mismatched |
| | −1 | ctgcagagaacctacgttca (SEQ ID NO: 388) | tgg | 43.308889 | 46.77630494 | Partially, |
| | | | | | | mismatched |
| | 1 | tccgtgactctccatacgag (SEQ ID NO: 389) | tgg | 42.3518877 | 64.95530799 | Yes |
| | 1 | atgaacgtaggttctctgca (SEQ ID NO: 390) | ggg | 41.9357474 | 65.89058318 | Partially, |
| | | | | | | mismatched |
| | −1 | gctctctcctgccagaaccg (SEQ ID NO: 391) | tgg | 39.9122374 | 53.48535523 | Yes |
| | −1 | agcatctgtaggttcctccg (SEQ ID NO: 392) | tgg | 32.9490905 | 60.90621662 | Yes |
| | 1 | taggttctctgcagggccca (SEQ ID NO: 393) | agg | 31.8896396 | 58.07516431 | Yes |
| | −1 | ctcgagtttgaccactcgta (SEQ ID NO: 394) | tgg | 31.0199135 | 56.19377776 | Yes |
| | −1 | accactcgtatggagagtca (SEQ ID NO: 395) | cgg | 29.8382482 | 67.75748604 | Yes |
| | −1 | ctctctcctgccagaaccgt (SEQ ID NO: 396) | ggg | 28.317125 | 40.78289164 | Yes |
| | −1 | ggcccagaggaaagtcggcc (SEQ ID NO: 397) | tgg | 27.118728 | 35.48158427 | Yes |
| | −1 | ctggaatgttccgttgacct (SEQ ID NO: 398) | tgg | 26.6891718 | 39.22415654 | Yes |
| | −1 | tggaatgttccgttgacctt (SEQ ID NO: 399) | ggg | 26.2257382 | 43.51741671 | Yes |
| | 1 | tctgcagggcccaaggtcaa (SEQ ID NO: 400) | cgg | 25.5217031 | 58.68215106 | Yes |
| | −1 | gcatctgtaggttcctccgt (SEQ ID NO: 401) | ggg | 23.7641164 | 64.09139455 | Yes |
| | −1 | ggatagatggtacatgtcat (SEQ ID NO: 402) | agg | 22.5719786 | 58.79313198 | Yes |
| | 1 | caaggtcaacggaacattcc (SEQ ID NO: 403) | agg | 22.5410529 | 45.79626069 | Yes |
| | 1 | ccgggccccacggttctggc (SEQ ID NO: 404) | agg | 22.1712727 | 46.55746509 | Yes |
| | 1 | atgacatgtaccatctatcc (SEQ ID NO: 405) | agg | 22.0305575 | 44.75201749 | Yes |
| | −1 | tctctcctgccagaaccgtg (SEQ ID NO: 406) | ggg | 20.8300787 | 67.22642591 | Yes |
| | −1 | ttcatgggcctccccctccc (SEQ ID NO: 407) | tgg | 20.4729882 | 41.28088943 | Yes |
| | 1 | tgtaccatctatccagggag (SEQ ID NO: 408) | ggg | 20.034197 | 58.18580496 | Yes |
| | 1 | agggggaggcccatgaacgt (SEQ ID NO: 409) | agg | 18.9738229 | 63.6706367 | Partially, |
| | | | | | | mismatched |
| | −1 | ataggagctccgggagctgc (SEQ ID NO: 410) | agg | 18.8988426 | 49.6265444 | Yes |
| | 1 | attccaggccgactttcctc (SEQ ID NO: 411) | tgg | 18.831583 | 31.78584288 | Yes |
| | −1 | taggttcctccgtgggtggc (SEQ ID NO: 412) | agg | 18.6401946 | 33.90580348 | Yes |
| | 1 | ttccaggccgactttcctct (SEQ ID NO: 413) | ggg | 18.3037756 | 51.86413845 | Yes |
| | 1 | atgtaccatctatccaggga (SEQ ID NO: 414) | ggg | 17.9123846 | 65.85919053 | Yes |
| | 1 | tgacatgtaccatctatcca (SEQ ID NO: 415) | ggg | 17.6685576 | 64.17145284 | Yes |
| | −1 | ccagaaccgtggggcccggc (SEQ ID NO: 416) | tgg | 17.5233952 | 26.25436916 | Yes |
| | −1 | cctgccagaaccgtggggcc (SEQ ID NO: 417) | cgg | 17.4086492 | 46.57815807 | Yes |
| | −1 | cagaaccgtggggcccggct (SEQ ID NO: 418) | ggg | 17.2023021 | 40.47921169 | Yes |
| | 1 | ggaggaacctacagatgctt (SEQ ID NO: 419) | cgg | 17.1023595 | 52.37081136 | Yes |
| | 1 | ccagccgggccccacggttc (SEQ ID NO: 420) | tgg | 16.4944144 | 31.16041254 | Yes |
| | 1 | gtaccatctatccagggagg (SEQ ID NO: 421) | ggg | 16.2348456 | 59.07845567 | Yes |
| | −1 | gtacatgtcataggagctcc (SEQ ID NO: 422) | ggg | 15.844385 | 54.15898367 | Yes |
| | 1 | catgtaccatctatccaggg (SEQ ID NO: 423) | agg | 15.7311139 | 71.77109475 | Yes |
| | 1 | ccatctatccagggaggggg (SEQ ID NO: 424) | agg | 15.6443179 | 45.35911519 | Yes |
| | −1 | ggtacatgtcataggagctc (SEQ ID NO: 425) | cgg | 15.5683706 | 46.55891931 | Yes |
| | −1 | ggcagggcccagaggaaagt (SEQ ID NO: 426) | cgg | 15.4697357 | 49.6486146 | Yes |
| | −1 | cctccccctccctggataga (SEQ ID NO: 427) | tgg | 15.4331486 | 37.06286031 | Yes |
| | −1 | tctgtaggttcctccgtggg (SEQ ID NO: 428) | tgg | 14.5487998 | 63.6725736 | Yes |
| | −1 | ctgtgacagaaacaagcagt (SEQ ID NO: 429) | ggg | 14.167692 | 70.81083421 | Yes |
| | −1 | gaaggtttctcatatagacc (SEQ ID NO: 430) | tgg | 13.5866209 | 58.08814131 | Yes |
| | −1 | gctccgggagctgcaggaca (SEQ ID NO: 431) | agg | 13.2586199 | 56.31871721 | Yes |
| | 1 | gcttgtttctgtcacaggtg (SEQ ID NO: 432) | agg | 13.1370879 | 59.34421277 | Yes |
| | 1 | tgaccttgtcctgcagctcc (SEQ ID NO: 433) | cgg | 13.1335213 | 44.62611872 | Yes |
| | −1 | aggttcctccgtgggtggca (SEQ ID NO: 434) | ggg | 12.996601 | 51.90925589 | Yes |
| | −1 | cctgtgacagaaacaagcag (SEQ ID NO: 435) | tgg | 12.9022335 | 75.38433487 | Yes |
| | 1 | ctcagcccagccgggcccca (SEQ ID NO: 436) | cgg | 12.6112714 | 45.24662974 | Yes |
| | 1 | ctgggccctgccacccacgg (SEQ ID NO: 437) | agg | 12.2718516 | 60.36188811 | Yes |
| | 1 | ccactgcttgtttctgtcac (SEQ ID NO: 438) | agg | 12.269857 | 46.38469641 | Yes |
| | 1 | ccttctctctcagcccagcc (SEQ ID NO: 439) | ggg | 12.0416884 | 50.40044061 | Yes |
| | −1 | gaaagagccgaagcatctgt (SEQ ID NO: 440) | agg | 11.8256907 | 64.81372513 | Yes |
| | 1 | accttctctctcagcccagc (SEQ ID NO: 441) | cgg | 11.5867593 | 40.49696239 | Yes |
| | −1 | cccggctgggctgagagaga (SEQ ID NO: 442) | agg | 10.6934148 | 48.09196456 | Yes |
| | −1 | ccgtgggtggcagggcccag (SEQ ID NO: 443) | agg | 10.0379832 | 59.97182871 | Yes |
| | 1 | cctctgggccctgccaccca (SEQ ID NO: 444) | cgg | 9.6833026 | 49.04004993 | Yes |
| Exon 5 | −1 | tactcaccggtttcggagct (SEQ ID NO: 445) | tgg | 66.0373089 | 31.1778444 | Partially, |
| | | | | | | mismatched |
| | 1 | actgaaccaagctccgaaac (SEQ ID NO: 446) | cgg | 60.8220429 | 52.51565523 | Partially, |
| | | | | | | mismatched |
| | −1 | ggtttcggagcttggttcag (SEQ ID NO: 447) | tgg | 32.2149711 | 58.99444897 | Partially, |
| | | | | | | mismatched |
| | −1 | ggttctgtactcaccggttt (SEQ ID NO: 448) | cgg | 24.3050155 | 37.1347178 | Yes |
| | −1 | gtttcggagcttggttcagt (SEQ ID NO: 449) | ggg | 23.469839 | 55.43700438 | Partially, |
| | | | | | | mismatched |
| | −1 | ggtgaaggccaactatttga (SEQ ID NO: 450) | agg | 20.7173391 | 43.55714515 | Yes |
| | 1 | taggaaacccttcaaatagt (SEQ ID NO: 451) | tgg | 20.5263725 | 42.8174115 | Yes |
| | −1 | gtgaaggccaactatttgaa (SEQ ID NO: 452) | ggg | 19.9184363 | 51.3711373 | Yes |
| | −1 | gagcttggttcagtgggtga (SEQ ID NO: 453) | agg | 14.748832 | 56.36496631 | Yes |
| Exon 6 | −1 | aatcagaacatgcaggtgtc (SEQ ID NO: 454) | tgg | 47.5829677 | 38.43684338 | Partially, |
| | | | | | | mismatched |
| | −1 | tgttgcagcaccagcgatga (SEQ ID NO: 455) | agg | 38.6786342 | 50.79660146 | Partially, |
| | | | | | | mismatched |
| | −1 | atcagaacatgcaggtgtct (SEQ ID NO: 456) | ggg | 35.1210633 | 48.78038744 | No |
| | −1 | tcagaacatgcaggtgtctg (SEQ ID NO: 457) | ggg | 28.9464104 | 58.45282741 | No |
| | −1 | aggtcccaatcagaacatgc (SEQ ID NO: 458) | agg | 27.9159958 | 62.88478036 | Partially, |
| | | | | | | mismatched |
| | −1 | ctggggttacctggaagaag (SEQ ID NO: 459) | agg | 25.5617285 | 55.0454841 | Partially, |
| | | | | | | mismatched |
| | 1 | gacacctgcatgttctgatt (SEQ ID NO: 460) | ggg | 23.465292 | 39.2429696 | Partially, |
| | | | | | | mismatched |
| | −1 | tgcaggtgtctggggttacc (SEQ ID NO: 461) | tgg | 22.2331694 | 43.36555514 | Partially, |
| | | | | | | mismatched |
| | 1 | agacacctgcatgttctgat (SEQ ID NO: 462) | tgg | 20.9169398 | 47.97933144 | No |
| | −1 | gaagaggatgatgaccactg (SEQ ID NO: 463) | agg | 18.7676197 | 77.02928766 | Yes |
| | 1 | tgttctgattgggacctcag (SEQ ID NO: 464) | tgg | 17.4872067 | 69.16400607 | Partially, |
| | | | | | | mismatched |
| | 1 | tcttctttctccttcatcgc (SEQ ID NO: 465) | tgg | 16.0977219 | 41.84089734 | Yes |
| | −1 | agcgatgaaggagaaagaag (SEQ ID NO: 466) | agg | 12.9288675 | 58.06787307 | Yes |
| | −1 | gatgaaggagaaagaagagg (SEQ ID NO: 467) | agg | 9.0025407 | 64.0334756 | Yes |
| | −1 | agaagaggaggaggatgaag (SEQ ID NO: 468) | agg | 7.740379 | 55.36986076 | Partially, |
| | | | | | | mismatched |
| | −1 | gaaggagaaagaagaggagg (SEQ ID NO: 469) | agg | 5.7840406 | 47.34603095 | Yes |
| Exon 7 | 1 | cagggaacagaacagtgaac (SEQ ID NO: 470) | agg | 32.5507395 | 47.50932357 | No |
| | 1 | ttctacagatgctgttgtaa (SEQ ID NO: 471) | tgg | 32.2631454 | 41.87399496 | No |
| | 1 | agggaacagaacagtgaaca (SEQ ID NO: 472) | ggg | 31.4985129 | 67.52387829 | No |
| | 1 | gaacagaacagtgaacaggg (SEQ ID NO: 473) | agg | 22.6519815 | 76.14156075 | No |
| | 1 | agaacagtgaacagggaggt (SEQ ID NO: 474) | agg | 22.0834895 | 61.08004299 | No |
| | −1 | ttcactgttctgttccctgc (SEQ ID NO: 475) | agg | 19.6091139 | 37.03196928 | No |
| | 1 | taatggaccaagagcctgca (SEQ ID NO: 476) | ggg | 16.4804239 | 59.69244865 | No |
| | −1 | ttctgttccctgcaggctct (SEQ ID NO: 477) | tgg | 15.3092828 | 36.67539547 | No |
| | 1 | gtaatggaccaagagcctgc (SEQ ID NO: 478) | agg | 13.746172 | 59.05570974 | No |
| Exon 8 | 1 | aacagatatcatcgtgtaca (SEQ ID NO: 479) | cgg | 45.2890654 | 62.71699919 | Yes |
| | 1 | gtacacgatgatatctgttg (SEQ ID NO: 480) | ggg | 44.9529219 | 61.98329108 | Yes |
| | 1 | gtgtacacgatgatatctgt (SEQ ID NO: 481) | tgg | 44.2078237 | 57.17566505 | Yes |
| | 1 | tgtacacgatgatatctgtt (SEQ ID NO: 482) | ggg | 43.5868555 | 42.60355046 | Yes |
| | −1 | tacacgatgatatctgttgg (SEQ ID NO: 483) | ggg | 43.2709289 | 72.67309996 | Yes |
| | −1 | tgtgcatatgtcacctcctg (SEQ ID NO: 484) | agg | 33.2275797 | 57.88346978 | Partially, |
| | | | | | | mismatched |
| | −1 | gtgcatatgtcacctcctga (SEQ ID NO: 485) | ggg | 25.8787959 | 63.17946944 | Partially, |
| | | | | | | mismatched |
| | 1 | atatctgttgggggtgtctt (SEQ ID NO: 486) | ggg | 17.9587785 | 34.80286512 | Yes |
| | −1 | gatatctgttgggggtgtct (SEQ ID NO: 487) | tgg | 17.5950576 | 33.17325643 | Yes |
| | 1 | ctctgatgaacaagaccctc (SEQ ID NO: 488) | agg | 14.5402588 | 50.15205354 | Yes |
| | 1 | aaatcactcgcccttctcag (SEQ ID NO: 489) | agg | 14.2544365 | 65.66875202 | Yes |
| | −1 | gtgtcttgggcctctgagaa (SEQ ID NO: 490) | ggg | 13.9295958 | 49.88141773 | Yes |
| | 1 | tgatgaacaagaccctcagg (SEQ ID NO: 491) | agg | 13.7606138 | 68.32294117 | Yes |
| | −1 | ggtgtcttgggcctctgaga (SEQ ID NO: 492) | agg | 13.7195437 | 37.36705244 | Yes |
| 3′ UTR | −1 | gtctaagtgccgtgttaaga (SEQ ID NO: 493) | ggg | 85.7686816 | 45.94761347 | Partially, |
| | | | | | | mismatched |
| | −1 | tgtctaagtgccgtgttaag (SEQ ID NO: 494) | agg | 81.7349064 | 47.21635913 | Partially, |
| | | | | | | mismatched |
| | 1 | gagggagcctcttgtatgtt (SEQ ID NO: 495) | tgg | 76.8782493 | 35.37362392 | Yes |
| | 1 | gcccacctctccaacctaac (SEQ ID NO: 496) | tgg | 76.2988801 | 39.87036309 | Yes |
| | 1 | attgcagcctcaagtagact (SEQ ID NO: 497) | agg | 72.4829698 | 57.0478991 | Yes |
| | 1 | gactaggaagtaagccagtt (SEQ ID NO: 498) | agg | 72.2442137 | 46.00607944 | Yes |
| | 1 | aggaagtaagccagttaggt (SEQ ID NO: 499) | tgg | 71.9042489 | 41.7340746 | Yes |
| | 1 | caagaggctccctcttaaca (SEQ ID NO: 500) | cgg | 71.7120533 | 55.36293745 | No |
| | 1 | gtaagccagttaggttggag (SEQ ID NO: 501) | agg | 71.54855 | 55.56736427 | Yes |
| | −1 | aggctgaaagctagtctgag (SEQ ID NO: 502) | ggg | 68.1179251 | 65.42780553 | Partially, |
| | | | | | | mismatched |
| | 1 | cttacttcctagtctacttg (SEQ ID NO: 503) | agg | 67.8417065 | 55.35650233 | Yes |
| | −1 | gaaggctgaaagctagtctg (SEQ ID NO: 504) | agg | 67.5454207 | 60.21593691 | Partially, |
| | | | | | | mismatched |
| | −1 | aaggctgaaagctagtctga (SEQ ID NO: 505) | ggg | 65.6494179 | 54.96128006 | Partially, |
| | | | | | | mismatched |
| | −1 | ctgaaagctagtctgagggg (SEQ ID NO: 506) | agg | 61.702876 | 67.32684796 | Yes |
| | 1 | ttgaggctgcaatcacactg (SEQ ID NO: 507) | agg | 61.31892 | 74.74770129 | Yes |
| | −1 | accttagacatttgtaagca (SEQ ID NO: 508) | agg | 60.6800351 | 52.82731131 | Yes |
| | −1 | gccagttaggttggagaggt (SEQ ID NO: 509) | ggg | 59.3258049 | 46.75048202 | Yes |
| | −1 | tgggcaggggtcaagtgaaa (SEQ ID NO: 510) | tgg | 56.9295509 | 35.45301848 | Yes |
| | 1 | cacaattccaaacatacaag (SEQ ID NO: 511) | agg | 55.9615864 | 61.51772376 | Yes |
| | −1 | ggggaggtggaacagcatga (SEQ ID NO: 512) | ggg | 54.6131044 | 69.50227076 | Yes |
| | −1 | aggggaggtggaacagcatg (SEQ ID NO: 513) | agg | 51.8017596 | 53.59962361 | Yes |
| | −1 | gggcagacatgtttatttga (SEQ ID NO: 514) | agg | 51.3386924 | 36.96875759 | Yes |
| | 1 | agccagttaggttggagagg (SEQ ID NO: 515) | tgg | 49.6070841 | 51.45946584 | Yes |
| | −1 | aaagctagtctgaggggagg (SEQ ID NO: 516) | tgg | 48.5212124 | 47.86005338 | Yes |
| | −1 | caagtgaaatggagaattgt (SEQ ID NO: 517) | ggg | 48.5020589 | 47.48781186 | No |
| | −1 | aggtggaacagcatgaggga (SEQ ID NO: 518) | agg | 47.5710532 | 63.15679382 | Yes |
| | −1 | cagacatgtttatttgaagg (SEQ ID NO: 519) | agg | 45.7764956 | 57.67541929 | Yes |
| | 1 | agacatgtttatttgaagga (SEQ ID NO: 520) | ggg | 43.9897466 | 54.03800771 | Yes |
| | −1 | gttaggttggagaggtgggc (SEQ ID NO: 521) | agg | 42.1263881 | 37.29893803 | Yes |
| | −1 | tggaacagcatgagggaagg (SEQ ID NO: 522) | tgg | 41.6545549 | 56.47796615 | Yes |
| | −1 | caggagacaactttggatca (SEQ ID NO: 523) | ggg | 39.7134765 | 58.64526635 | No |
| | −1 | tcaagtgaaatggagaattg (SEQ ID NO: 524) | tgg | 39.6216546 | 60.10255012 | No |
| | 1 | aataaacatgtctgccctca (SEQ ID NO: 525) | tgg | 34.8252947 | 44.61062305 | No |
| | −1 | agctgctggtacattggagc (SEQ ID NO: 526) | tgg | 34.6673035 | 47.54986942 | Partially, |
| | | | | | | mismatched |
| | −1 | taagttttactgctgacaga (SEQ ID NO: 527) | agg | 34.6623548 | 59.61364644 | Yes |
| | −1 | taggttggagaggtgggcag (SEQ ID NO: 528) | ggg | 33.4801222 | 57.42383612 | Yes |
| | −1 | gagaggcacgttcagatttg (SEQ ID NO: 529) | tgg | 29.0463529 | 48.51480044 | Partially, |
| | | | | | | mismatched |
| | −1 | ttaggttggagaggtgggca (SEQ ID NO: 530) | ggg | 28.4425748 | 47.83669021 | Yes |
| | −1 | acgttcagatttgtggtgtg (SEQ ID NO: 531) | agg | 27.7003306 | 60.41289962 | Partially, |
| | | | | | | mismatched |
| | 1 | aacagccctgtctcaaaact (SEQ ID NO: 532) | ggg | 26.908913 | 52.14224733 | Partially, |
| | | | | | | mismatched |
| | −1 | gacatttgtaagcaagggag (SEQ ID NO: 533) | agg | 25.1192321 | 53.53298664 | Partially, |
| | | | | | | mismatched |
| | −1 | ggcaacccagttttgagaca (SEQ ID NO: 534) | ggg | 24.7611803 | 56.99338082 | Partially, |
| | | | | | | mismatched |
| | −1 | ctccctagaagatcccctca (SEQ ID NO: 535) | agg | 24.1212625 | 62.89687787 | No |
| | 1 | ggccttgaggggatcttcta (SEQ ID NO: 536) | ggg | 22.4156621 | 43.83720683 | No |
| | 1 | aaggcgtgagtctgcatctt (SEQ ID NO: 537) | agg | 22.2561824 | 32.69162834 | Partially, |
| | | | | | | mismatched |
| | 1 | caccacagtcaggccttgag (SEQ ID NO: 538) | ggg | 21.8766273 | 61.63740523 | Yes |
| | −1 | atcccctcaaggcctgactg (SEQ ID NO: 539) | tgg | 19.7079327 | 65.45404763 | No |
| | −1 | tggcaacccagttttgagac (SEQ ID NO: 540) | agg | 19.5911178 | 50.55126578 | Partially, |
| | | | | | | mismatched |
| | −1 | gagaattgtgggctaagcaa (SEQ ID NO: 541) | agg | 19.0957508 | 57.41063719 | No |
| | 1 | aggcgtgagtctgcatctta (SEQ ID NO: 542) | ggg | 19.0373883 | 44.82221526 | Partially, |
| | | | | | | mismatched |
| | 1 | aggccttgaggggatcttct (SEQ ID NO: 543) | agg | 18.9636057 | 40.84557057 | No |
| | 1 | gcaggagacaactttggatc (SEQ ID NO: 544) | agg | 18.3112446 | 40.90351554 | Yes |
| | −1 | tcatgggcaggagacaactt (SEQ ID NO: 545) | tgg | 18.2856344 | 58.0617284 | Yes |
| | 1 | agctccaatgtaccagcagc (SEQ ID NO: 546) | tgg | 18.1416407 | 43.24124596 | Partially, |
| | | | | | | mismatched |
| | −1 | ggcctgactgtggtgctcat (SEQ ID NO: 547) | ggg | 18.0906072 | 46.09773639 | Yes |
| | 1 | cccttgcttacaaatgtcta (SEQ ID NO: 548) | agg | 17.4941922 | 48.85914721 | Partially, |
| | | | | | | mismatched |
| | −1 | tgactgtggtgctcatgggc (SEQ ID NO: 549) | agg | 16.8588557 | 45.3066133 | Yes |
| | −1 | gagtgtgttttctctccagc (SEQ ID NO: 550) | agg | 16.8397981 | 52.99714076 | No |
| | 1 | agcaccacagtcaggccttg (SEQ ID NO: 551) | agg | 16.725956 | 61.85842884 | Yes |
| | 1 | ctaaggtccccactgcctgc (SEQ ID NO: 552) | tgg | 16.4850162 | 46.35050595 | Yes |
| | −1 | aggcctgactgtggtgctca (SEQ ID NO: 553) | tgg | 15.9475718 | 39.75667041 | Yes |
| | 1 | tgcccatgagcaccacagtc (SEQ ID NO: 554) | agg | 14.7333679 | 44.70471004 | Yes |
| | 1 | caacagccctgtctcaaaac (SEQ ID NO: 555) | tgg | 14.6632947 | 34.68933751 | Yes |
| | 1 | gcaccacagtcaggccttga (SEQ ID NO: 556) | ggg | 14.4012919 | 53.73829722 | Yes |
| | −1 | ccttagacatttgtaagcaa (SEQ ID NO: 557) | ggg | 14.2698043 | 58.63277422 | Yes |
| | −1 | ttttctctccagcaggcagt (SEQ ID NO: 558) | ggg | 13.820459 | 43.1256203 | No |
| | 1 | gattccagctgctggtacat (SEQ ID NO: 559) | tgg | 12.8461481 | 36.60884306 | Yes |
| | 1 | tttctctccagcaggcagtg (SEQ ID NO: 560) | ggg | 12.447256 | 66.39353334 | No |
| | 1 | cgccttcagattccagctgc (SEQ ID NO: 561) | tgg | 12.0110431 | 46.85022416 | Partially, |
| | | | | | | mismatched |
| | −1 | gttttctctccagcaggcag (SEQ ID NO: 562) | tgg | 11.5641021 | 47.76062242 | No |
| | 1 | taccagcagctggaatctga (SEQ ID NO: 563) | agg | 11.2821164 | 58.57564638 | Yes |

Cpf1 (Cas12)

| | | | | | |
|---|---|---|---|---|---|
| Promoter | −1 | gcgccctgcccatgcagtttaca (SEQ ID NO: 564) | tttg | 15.3169456 | Yes |
| | −1 | catgttgaccacatcatgggagg (SEQ ID NO: 565) | ttta | 15.298257 | Yes |
| | −1 | taccttgcatgaggcccagtggg (SEQ ID NO: 566) | tttc | 14.1642416 | Yes |
| Signal | −1 | gagaaggactcaccctcatgtgg (SEQ ID NO: 567) | tttg | 48.6546342 | Yes |
| peptide + | 1 | cagggttcttcttgctgcagggg (SEQ ID NO: 568) | tttc | 17.9146425 | Yes |
| Exon 2 | 1 | tttccagggttcttcttgctgca (SEQ ID NO: 569) | tttc | 16.1254393 | Yes |
| Exon 3 | 1 | aggacactttgcacctcattgga (SEQ ID NO: 570) | ttta | 85.341312 | Yes |
| | 1 | cacctcattggagagcaccatga (SEQ ID NO: 571) | tttg | 38.7284003 | Yes |
| | −1 | accaggggacctgggtgggccag (SEQ ID NO: 572) | tttc | 17.9476219 | Partially, |
| | | | | | mismatched |
| | −1 | ctgtggactcctaggaagagagg (SEQ ID NO: 573) | tttt | 17.9275832 | Yes |
| | −1 | tgtggactcctaggaagagaggt (SEQ ID NO: 574) | tttc | 17.8542264 | Yes |
| | 1 | agcacttccttctgcacagagaa (SEQ ID NO: 575) | tttc | 17.5260664 | Yes |
| Exon 4 | −1 | tcatatagacctggaggagaaga (SEQ ID NO: 576) | tttc | 92.9065867 | Partially, |
| | | | | | mismatched |
| | −1 | accactcgtatggagagtcacgg (SEQ ID NO: 577) | tttg | 39.5758627 | Yes |
| | 1 | cgtgactctccatacgagtggtc (SEQ ID NO: 578) | tttc | 39.261553 | Yes |
| | 1 | tgtcacaggtgaggaaaccccat (SEQ ID NO: 579) | tttc | 24.4341784 | Yes |
| | 1 | ctctgggccctgccacccacgga (SEQ ID NO: 580) | tttc | 16.4354341 | Yes |
| | −1 | ctcacctgtgacagaaacaagca (SEQ ID NO: 581) | tttc | 13.9866894 | Yes |
| Exon 5 | −1 | aagggtttcctagaacacgagac (SEQ ID NO: 582) | tttg | 39.2845023 | Partially, |
| | | | | | mismatched |
| | 1 | ctcacctctctcctgtctcgtgt (SEQ ID NO: 583) | tttc | 37.6898834 | Partially, |
| | | | | | mismatched |
| | −1 | ctagaacacgagacaggagagag (SEQ ID NO: 584) | tttc | 28.0859682 | Partially, |
| | | | | | mismatched |
| | −1 | ggagcttggttcagtgggtgaag (SEQ ID NO: 585) | tttc | 22.0568055 | Partially, |
| | | | | | mismatched |
| Exon 6 | −1 | tttgttgcagcaccagcgatgaa (SEQ ID NO: 586) | tttt | 62.9351594 | Partially, |
| | | | | | mismatched |
| | −1 | gttgcagcaccagcgatgaagga (SEQ ID NO: 587) | tttt | 62.6681533 | Partially, |
| | | | | | mismatched |
| | −1 | ttgcagcaccagcgatgaaggag (SEQ ID NO: 588) | tttg | 62.6337149 | Partially, |
| | | | | | mismatched |
| | −1 | tgttgcagcaccagcgatgaagg (SEQ ID NO: 589) | tttt | 62.0406464 | Partially, |
| | | | | | mismatched |
| | −1 | ttgttgcagcaccagcgatgaag (SEQ ID NO: 590) | tttt | 48.5104494 | Partially, |
| | | | | | mismatched |
| | 1 | tccttcatcgctggtgctgcaac (SEQ ID NO: 591) | tttc | 38.5906107 | Partially, |
| | | | | | mismatched |
| Exon 8 | −1 | gatcagggctcagcatttggaag (SEQ ID NO: 592) | tttg | 47.547249 | Partially, |
| | | | | | mismatched |
| | −1 | gaagttccgtgtacacgatgata (SEQ ID NO: 593) | tttg | 33.0685405 | Yes |
| | 1 | cctctctccaggactctgatgaa (SEQ ID NO: 594) | tttc | 16.3395078 | Yes |
| | −1 | tctgtgtgaaaacgcagtgattc (SEQ ID NO: 595) | tttc | 15.1976376 | Yes |
| | −1 | ctctgtgtgaaaacgcagtgatt (SEQ ID NO: 596) | tttt | 15.1682898 | Yes |
| | −1 | tctctgtgtgaaaacgcagtgat (SEQ ID NO: 597) | tttt | 15.1554695 | Yes |
| | 1 | acacagagaaaaatcactcgccc (SEQ ID NO: 598) | tttc | 14.9951438 | Yes |
| | 1 | cacacagagaaaaatcactcgcc (SEQ ID NO: 599) | tttt | 14.7512901 | Yes |
| 3′ UTR | 1 | acttgacccctgcccacctctcc (SEQ ID NO: 600) | tttc | 94.5211894 | Yes |
| | 1 | agccttctgtcagcagtaaaact (SEQ ID NO: 601) | tttc | 91.3387595 | Partially, |
| | | | | | mismatched |
| | −1 | gaattgtgagttcctcagtgtga (SEQ ID NO: 602) | tttg | 91.0259628 | Yes |
| | −1 | ctgctgacagaaggctgaaagct (SEQ ID NO: 603) | ttta | 90.2140047 | Partially, |
| | | | | | mismatched |
| | −1 | taagcaagggagaggcacgttca (SEQ ID NO: 604) | tttg | 85.9786723 | Partially, |
| | | | | | mismatched |
| | −1 | aaggagggaaaactacattgaaa (SEQ ID NO: 605) | tttg | 85.3860235 | Partially, |
| | | | | | mismatched |
| | 1 | ccctccttcaaataaacatgtct (SEQ ID NO: 606) | tttt | 84.8341329 | Partially, |
| | | | | | mismatched |
| | −1 | tttgaaggagggaaaactacatt (SEQ ID NO: 607) | ttta | 83.8276899 | Partially, |
| | | | | | mismatched |
| | 1 | cctccttcaaataaacatgtctg (SEQ ID NO: 608) | tttc | 75.9396772 | Partially, |
| | | | | | mismatched |
| | 1 | aatgtagttttccctccttcaaa (SEQ ID NO: 609) | tttc | 75.4129909 | Partially, |
| | | | | | mismatched |
| | 1 | ttaaaataatttcaatgtagttt (SEQ ID NO: 610) | tttt | 63.1016567 | Partially, |
| | | | | | mismatched |
| | −1 | actgctgacagaaggctgaaagc (SEQ ID NO: 611) | tttt | 62.1351189 | Partially, |
| | | | | | mismatched |
| | −1 | aaaaaatatataagttttactgc (SEQ ID NO: 612) | tttt | 59.4114203 | Yes |
| | 1 | aaataatttcaatgtagttttcc (SEQ ID NO: 613) | ttta | 58.4381586 | No |
| | 1 | taaaataatttcaatgtagtttt (SEQ ID NO: 614) | tttt | 47.773251 | Partially, |
| | | | | | mismatched |
| | −1 | gatcagggctcagcatttggaag (SEQ ID NO: 615) | tttg | 47.547249 | Partially, |
| | | | | | mismatched |
| | 1 | aaaataatttcaatgtagttttc (SEQ ID NO: 616) | tttt | 45.4837969 | Partially, |
| | | | | | mismatched |
| | −1 | aaaaatatataagttttactgct (SEQ ID NO: 617) | ttta | 45.0377091 | Yes |
| | −1 | tggtgtgaggaagagcgatgccc (SEQ ID NO: 618) | tttg | 32.731087 | Yes |
| | 1 | cttagcccacaattctccatttc (SEQ ID NO: 619) | tttg | 24.2858931 | No |
| | −1 | ctctccagcaggcagtggggacc (SEQ ID NO: 620) | tttt | 17.8012221 | No |
| | −1 | tctccagcaggcagtggggacct (SEQ ID NO: 621) | tttc | 16.3318587 | Yes |
| | −1 | gagacagggctgttgtctcccta (SEQ ID NO: 622) | tttt | 15.2323502 | Yes |
| | −1 | agacagggctgttgtctccctag (SEQ ID NO: 623) | tttg | 13.2493833 | Yes |

Also disclosed are methods of disrupting KIR2DL2 and/or KIR2DL3 expression in T cells ex vivo while effectively expressing chimeric receptors. Therefore, disclosed herein is a chimeric cell expressing a chimeric receptor, wherein the chimeric receptor is encoded by a transgene, and wherein the transgene is inserted in the genome of the cell at a location that disrupts expression or activity of an endogenous KIR2DL2 and/or KIR2DL3 protein.

In other embodiments, the subject is treated with a KIR2DL2 and/or KIR2DL3 inhibitor prior to, during, or after treatment with adoptive cell immunotherapy. For example, the KIR2DL2 and/or KIR2DL3 inhibitor can be a blocking antibody that binds KIR2DL2 and/or KIR2DL3 Ig domains and blocks its binding to an HLA-C1. In some embodiments, the blocking antibody binds an HLA-C1 and blocks its binding to KIR2DL3 without blocking its binding to TCRs. Soluble receptors, such as KIR2DL2 and/or KIR2DL3 fragments or HLA-C fragments that block this interaction are also contemplated for use in the disclosed methods. The structure or human KIR2DL2 and/or KIR2DL3 and its binding to HLA-Cw3 is described in Boyington, J C, et al. Nature 2000 405:537-43, which is incorporated by reference for the teaching of these binding sites.

### Chimeric Antigen Receptors (CAR)

In particular embodiments, the immune effector cells with ablated KIR2DL2 and/or KIR2DL3 are engineered to express a chimeric antigen receptor (CAR) polypeptide. CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). The disclosed CAR is generally made up of three domains: an ectodomain, a transmembrane domain, and an endodomain. The ectodomain comprises the recognition domain. It also optionally contains a signal peptide (SP) so that the CAR can be glycosylated and anchored in the cell membrane of the immune effector cell. The transmembrane domain (TD), as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

Additional CAR constructs are described, for example, in Fresnak A D, et al. Engineered T cells: the promise and challenges of cancer immunotherapy. Nat Rev Cancer. 2016 Aug. 23; 16 (9): 566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or SCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen. Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. SCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIy, FcγRIIIy, FcεRIβ (FCERIB), and FcεRIy (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD33 chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., anti-CD123 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the recognition domain is a single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target two different antigens. Also disclosed are CARs designed to work only in conjunction with another CAR that binds a different antigen, such as a tumor antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRα, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, TIM3, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### Nucleic Acids and Vectors

Also disclosed are polynucleotides and polynucleotide vectors encoding the disclosed chimeric receptors. Also disclosed are oligonucleotides for use in inserting the chimeric receptors into the genome of a T cell at a site that will disrupt Sirt2 expression or activity.

Nucleic acid sequences encoding the disclosed chimeric receptors, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

### Immune Effector Cells

Also disclosed are immune effector cells that are engineered to express the disclosed chimeric receptors. These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{C} cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of a and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

### Therapeutic Methods

Immune effector cells expressing the disclosed chimeric receptors can elicit an anti-tumor immune response against cancer cells. The anti-tumor immune response elicited by the disclosed chimeric cells may be an active or a passive immune response. In addition, the immune response may be part of an adoptive immunotherapy approach in which chimeric cells induce an immune response specific to the target antigen.

Adoptive transfer of immune effector cells expressing chimeric receptors is a promising anti-cancer therapeutic. Following the collection of a patient's immune effector cells, the cells may be genetically engineered to express the disclosed chimeric receptors while ablating Sirt2 according to the disclosed methods, then infused back into the patient.

The disclosed chimeric effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat tumors. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed chimeric cells are administered to a patient in conjunction with (e.g., before, simultaneously or following) any number of relevant treatment modalities, including but not limited to thalidomide, dexamethasone, bortezomib, and lenalidomide. In further embodiments, the chimeric cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed chimeric cells can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed chimeric cells can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed chimeric cells can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent is a targeted agent, such as ibrutinib or idelalisib.

In some embodiments, such an additional therapeutic agent is an epigenetic modifier such as azacitdine or vidaza.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with chimeric cells for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C-C chemokine families. Suitable cytokines include cytokine derivatives, cytokine variants, cytokine fragments, and cytokine fusion proteins.

In some embodiments, a therapeutic agent for use in combination with chimeric cells for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with chimeric cells for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with chimeric cells for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed chimeric cells are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed chimeric cells are administered in combination with surgery.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

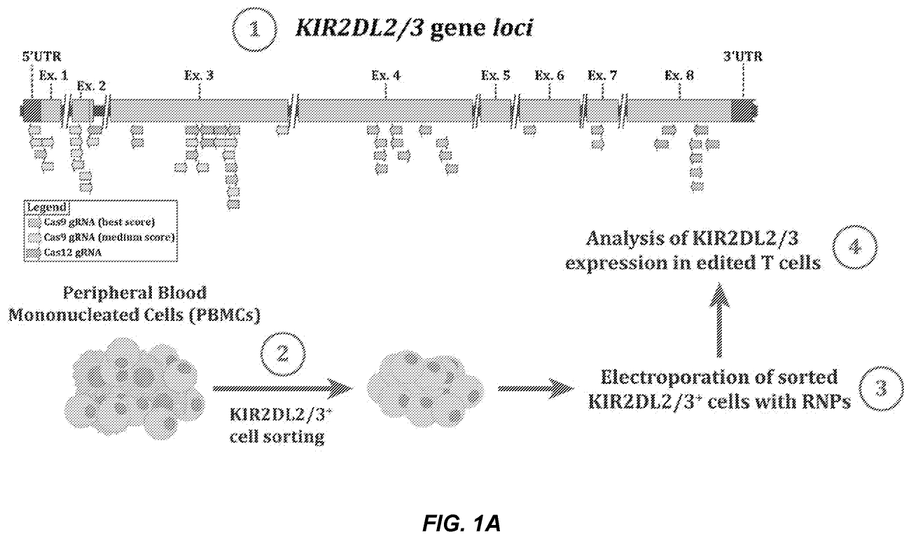

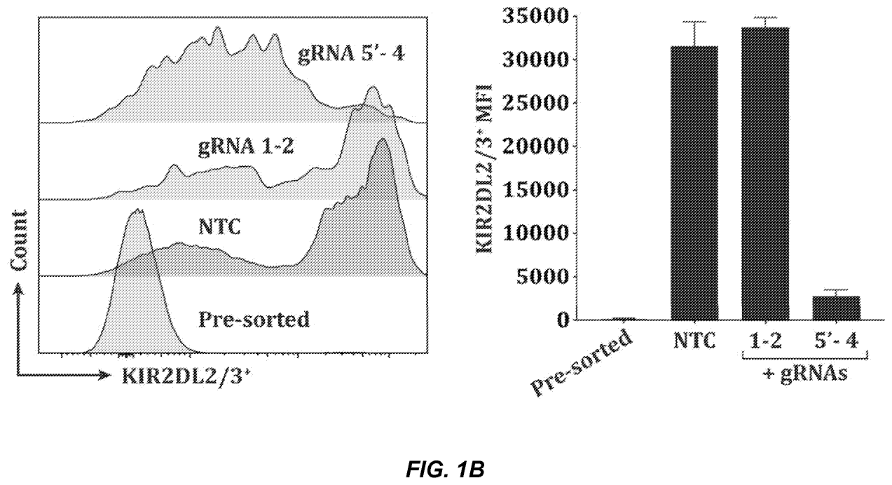

FIGS. 1A and 1B show design, validation, and testing of the strategy for KIR2DL2/3 abrogation in primary human T cells. A strategy was designed for the abrogation of KIR2DL2/3 expression during CAR-T cell manufacturing. It was validated using an in vivo model of NSG mice bearing acute lymphoblastic leukemia grafts. FIG. 1A is a schematic representation of the gene editing validation protocol. First, the KIR2DL2 and KIR2DL3 loci were analyzed for the presence of guide RNAs (gRNAs) with the highest cutting score (1). Due to the low expression of KIR2DL2 within PBMCs, the KIR2DL2/3+ population was enriched by fluorescent activated cell sorting (2). KIR2DL2+ T cells were then nucleofected (Neon Transfection System, Thermo Fisher Scientific) with either a non-targeting guide RNA (NTC gRNA) or a gRNA targeting KIR2DL2 exon 1 (3). KIR2DL2/3 expression was analyzed 48 hours after nucleofection by flow cytometry (4). FIG. 1B is a flow cytometry histogram of a representative experiment (left panel) and KIR2DL2/3 mean fluorescence intensity (MFI, right panel) comparisons between pre-sorted, unedited (NTC) and KIR2DL2/3-ablated T cells. Data are shown as a mean±SD of three independent experiments.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for enhancing anti-tumor activity of lymphocytes, comprising treating the lymphocytes with a KIR2DL2 and/or KIR2DL3 inhibitor or genetically modifying the lymphocytes to inhibit or ablate KIR2DL2 and/or KIR2DL3 expression.

2. A method, comprising:
(a) collecting lymphocytes from a subject with cancer;
(b) treating the lymphocytes with a KIR2DL2 and/or KIR2DL3 inhibitor or genetically modifying the lymphocytes to inhibit or ablate KIR2DL2 and/or KIR2DL3 expression.

3. The method of claim 1, wherein the KIR2DL2 and/or KIR2DL3 inhibitor is an siRNA, antisense, gRNA, or crRNA oligonucleotide.

4. The method of claim 1, wherein the lymphocytes are genetically modified by inserting a chimeric receptor into the genome of the cell at a location that disrupts expression or activity of an endogenous KIR2DL2 and/or KIR2DL3 protein.

5. The method cell of claim 5, wherein the chimeric receptor is a chimeric antigen receptor (CAR) polypeptide.

6. The method of claim 1, wherein the lymphocytes are selected from the group consisting of alpha-beta T cells, gamma-delta T cells, Natural Killer (NK) cells, Natural Killer T (NKT) cells, innate lymphoid cells (ILCs), cytokine induced killer (CIK) cells, cytotoxic T lymphocytes (CTLs), lymphokine activated killer (LAK) cells, and regulatory T (Treg) cells.

7. A therapeutic cell produced by the method of claim 1.

8. A method of providing an anti-cancer immunity in a subject, comprising administering to the subject an effective amount of the therapeutic cells of claim 6, thereby providing an anti-tumor immunity in the subject.

9. The method of claim 7, wherein the subject is HLA-C1+.

10. The method of claim 7, further comprising administering to the subject a checkpoint inhibitor.

11. The method of claim 9, wherein the checkpoint inhibitor comprises an anti-PD-1 antibody, anti-PD-L1 antibody, anti-CTLA-4 antibody, or a combination thereof.
